(19) United States
(12) Patent Application Publication
(10) Pub. No.: US 2025/0127811 A1
(43) Pub. Date: Apr. 24, 2025

(54) MODULATION OF TGF BETA SIGNALING IN GENETICALLY-MODIFIED EUKARYOTIC CELLS

(71) Applicant: Precision BioSciences, Inc., Durham, NC (US)

(72) Inventor: Aaron Martin, Carrboro, NC (US)

(73) Assignee: Precision BioSciences, Inc., Durham, NC (US)

(21) Appl. No.: 18/263,464

(22) PCT Filed: Jan. 28, 2022
(86) PCT No.: PCT/US2022/014218

§ 371 Date: Jul. 28, 2023

Related U.S. Application Data
(60) Provisional application No. 63/145,448, filed on Feb. 3, 2021.
(60) Provisional application No. 63/142,920, filed on Jan. 28, 2021.

(51) Int. Cl. A61K 35/17; A61K 40/11; A61K 40/31; A61K 40/42; C07K 14/71; C07K 14/725; C12N 5/0783; C12N 9/22; C12N 15/113; C12N 15/86

(52) U.S. Cl. CPC A61K 35/17; A61K 40/11; A61K 40/31; A61K 40/4215; C07K 14/7051; C07K 14/71; C12N 5/0636; C12N 9/22; C12N 15/1136; C12N 15/86; C12N 2310/14; C12N 2310/531; C12N 2510/00; C12N 2750/14143

(57) ABSTRACT

The application relates to the field of oncology, cancer immunotherapy, molecular biology and recombinant nucleic acid technology. In particular, the invention relates to genetically-modified eukaryotic cells comprising modulated TGF Beta signaling. The application further relates to the use of such genetically-modified eukaryotic cells for treating a disease, including cancer, in a subject.

### FIELD OF THE INVENTION

The application relates to the field of oncology, cancer immunotherapy, molecular biology and recombinant nucleic acid technology. In particular, the invention relates to genetically-modified eukaryotic cells comprising modulated TGF Beta signaling. The application further relates to the use of such genetically-modified eukaryotic cells for treating a disease, including cancer, in a subject.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jan. 25, 2022, is named P109070057WO00-SEQ-EPG, and is 13,996 bytes in size.

### BACKGROUND OF THE INVENTION

T cell adoptive immunotherapy is a promising approach for cancer treatment. The immunotherapy treatment methods disclosed herein utilize isolated human T cells that have been genetically-modified to enhance their specificity for a specific tumor associated antigen. Genetic modification may involve the expression of a chimeric antigen receptor or an exogenous T cell receptor to graft antigen specificity onto the T cell. By contrast to exogenous T cell receptors, chimeric antigen receptors derive their specificity from the variable domains of a monoclonal antibody. Thus, T cells expressing chimeric antigen receptors (CAR T cells) induce tumor immunoreactivity in a major histocompatibility complex non-restricted manner. T cell adoptive immunotherapy has been utilized as a clinical therapy for a number of cancers, including B cell malignancies (e.g., acute lymphoblastic leukemia, B cell non-Hodgkin lymphoma, acute myeloid leukemia, and chronic lymphocytic leukemia), multiple myeloma, neuroblastoma, glioblastoma, advanced gliomas, ovarian cancer, mesothelioma, melanoma, prostate cancer, pancreatic cancer, and others.

Despite its potential usefulness as a cancer treatment, adoptive immunotherapy with CAR T cells has been limited, in part, by expression of the endogenous T cell receptor on the cell surface. CAR T cells expressing an endogenous T cell receptor may recognize major and minor histocompatibility antigens following administration to an allogeneic patient, which can lead to the development of graft-versus-host-disease (GVHD). As a result, clinical trials have largely focused on the use of autologous CAR T cells, wherein a patient's T cells are isolated, genetically-modified to incorporate a chimeric antigen receptor, and then re-infused into the same patient. An autologous approach provides immune tolerance to the administered CAR T cells; however, this approach is constrained by both the time and expense necessary to produce patient-specific CAR T cells after a patient's cancer has been diagnosed.

Thus, it would be advantageous to develop “off the shelf” CAR T cells, prepared using T cells from a third party, healthy donor, that have reduced expression, or have no detectable cell-surface expression, of an endogenous T cell receptor (e.g., an alpha/beta T cell receptor) and do not initiate GVHD upon administration. Such products could be generated and validated in advance of diagnosis and could be made available to patients as soon as necessary. Therefore, a need exists for the development of allogeneic CAR T cells that lack an endogenous T cell receptor in order to prevent the occurrence of GVHD.

A significant hurdle to the development of allogenic adoptive cell therapies is inhibition of these genetically modified immune cells by the tumor microenvironment. The TGFβ family of ligands and receptors are important regulators of immune homeostasis and signal through the binding of a TGFβ ligand to the TGFβR1, TGFβR2, and TGFβR3 receptor tyrosine kinases. Upon binding to TGFβ, TGFβR2 phosphorylates TGFβR1 leading to activation of a signaling cascade. The canonical signaling pathway results in phosphorylation of Smad proteins by TGFβR1. Activation of this pathway can negatively affect immune cell proliferation and activation. Thus, there is an additional unmet need for an adoptive cell therapy that has modulated TGFβ signaling in order to maximize anti-tumor efficacy.

### SUMMARY OF THE INVENTION

In one aspect, the invention provides a genetically-modified eukaryotic cell comprising in its genome: (a) a nucleic acid sequence encoding a transforming growth factor beta-1 (TGFβ1, or TGFB-1) inhibitory agent; and (b) a nucleic acid sequence encoding an engineered antigen receptor.

In some embodiments, the TGFB-1 inhibitory agent reduces expression of TGFB-1 protein in the genetically-modified eukaryotic cell.

In some embodiments, the TGFB-1 inhibitory agent is an inhibitory nucleic acid molecule. In certain embodiments, the inhibitory nucleic acid molecule is an RNA interference molecule. In some embodiments, the RNA interference molecule is a short hairpin RNA (shRNA). In some embodiments, the RNA interference molecule is a small interfering RNA (siRNA). In some embodiments, the RNA interference molecule is a hairpin siRNA. In some embodiments, the RNA interference molecule is microRNA (miRNA). In some embodiments, the RNA interference molecule is a precursor miRNA.

In some embodiments, the RNA interference molecule is an miRNA-adapted shRNA (shRNAmiR). In certain embodiments, the shRNAmiR comprises, from 5′ to 3′: (a) a 5′ miR scaffold domain; (b) a 5′ miR basal stem domain; (c) a passenger strand; (d) a miR loop domain; (e) a guide strand; (f) a 3′ miR basal stem domain; and (g) a 3′ miR scaffold domain. In certain embodiments, the miR loop domain is a miR-30a loop domain, a miR-15 loop domain, a miR-16 loop domain, a miR-155 loop domain, a miR-22 loop domain, a miR-103 loop domain, or a miR-107 loop domain. In particular embodiments, the miR loop domain is a miR-30a loop domain. In some embodiments, the miR-30a loop domain comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 3. In particular embodiments, the miR-30a loop domain comprises a nucleic acid sequence set forth in SEQ ID NO: 3. In some embodiments, the shRNAmiR comprises a microRNA-E (miR-E) scaffold, a miR-30 (e.g., miR-30a) scaffold, a miR-15 scaffold, a miR-16 scaffold, a miR-155 scaffold, a miR-22 scaffold, a miR-103 scaffold, or a miR-107 scaffold. In certain embodiments, the shRNAmiR comprises a miR-E scaffold. In some embodiments, the shRNAmiR comprises a structure wherein: (a) the 5′ miR scaffold domain comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 4; (b) the 5′ miR basal stem domain comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 5; (c) the 3′ miR basal stem domain comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 6; and/or (d) the 3′ miR scaffold domain comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 7. In particular embodiments, the shRNAmiR comprises a structure wherein: (a) the 5′ miR scaffold domain comprises a nucleic acid sequence set forth in SEQ ID NO: 4; (b) the 5′ miR basal stem domain comprises a nucleic acid sequence set forth in SEQ ID NO: 5; (c) the 3′ miR basal stem domain comprises a nucleic acid sequence set forth in SEQ ID NO: 6; and (d) the 3′ miR scaffold domain comprises a nucleic acid sequence set forth in SEQ ID NO: 7. In certain embodiments, the shRNAmiR comprises a guide strand comprising a nucleic acid sequence set forth in SEQ ID NO: 8 and a passenger strand comprising a nucleic acid sequence set forth in SEQ ID NO: 9. In certain embodiments, the shRNAmiR comprises a guide strand comprising a nucleic acid sequence set forth in SEQ ID NO: 10 and a passenger strand comprising a nucleic acid sequence set forth in SEQ ID NO: 11. In certain embodiments, the shRNAmiR comprises a guide strand comprising a nucleic acid sequence set forth in SEQ ID NO: 12 and a passenger strand comprising a nucleic acid sequence set forth in SEQ ID NO: 13. In certain embodiments, the shRNAmiR comprises a guide strand comprising a nucleic acid sequence set forth in SEQ ID NO: 14 and a passenger strand comprising a nucleic acid sequence set forth in SEQ ID NO: 15. In certain embodiments, the shRNAmiR comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 16. In certain embodiments, the shRNAmiR comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 17. In certain embodiments, the shRNAmiR comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 18. In certain embodiments, the shRNAmiR comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 19. In particular embodiments, the shRNAmiR comprises a nucleic acid sequence set forth SEQ ID NO: 16. In particular embodiments, the shRNAmiR comprises a nucleic acid sequence set forth SEQ ID NO: 17. In particular embodiments, the shRNAmiR comprises a nucleic acid sequence set forth SEQ ID NO: 18. In particular embodiments, the shRNAmiR comprises a nucleic acid sequence set forth SEQ ID NO: 19.

In some embodiments, the TGFB-1 inhibitory agent is an engineered DNA-binding domain comprising a transcriptional repressor domain.

In some embodiments, expression of TGFB-1 protein by the genetically-modified eukaryotic cell is reduced by about 50% to about 99%, by about 55% to about 99%, by about 60% to about 99%, by about 65% to about 99%, by about 70% to about 99%, by about 75% to about 99%, by about 80% to about 99%, by about 85% to about 99%, by about 90% to about 99%, by about 95% to about 99%, by about 75% to about 95%, by about 80% to about 95%, by about 85% to about 95%, or by about 90% to about 95%, compared to a control cell.

In some embodiments, expression of TGFB-1 protein by the genetically-modified eukaryotic cell is reduced by about 50%, by about 55%, by about 60%, by about 65%, by about 70%, by about 75%, by about 80%, by about 81%, by about 82%, by about 83%, by about 84%, by about 85%, by about 86%, by about 87%, by about 88%, by about 89%, by about 90%, by about 91%, by about 92%, by about 93%, by about 94%, by about 95%, by about 96%, by about 97%, by about 98%, or by about 99%, compared to a control cell.

In some embodiments, the TGFB-1 inhibitory agent binds TGFB-1 protein produced by the genetically-modified eukaryotic cell.

In some embodiments, the TGFB-1 inhibitory agent is an antibody, or antibody fragment, having specificity for the TGFB-1 protein.

In some embodiments, the TGFB-1 inhibitory agent is a soluble TGFB receptor II (TGFBR2).

In some embodiments, the TGFB-1 inhibitory agent is secreted by the genetically-modified eukaryotic cell.

In some embodiments, the nucleic acid encoding the TGFB-1 inhibitory agent is positioned within a target gene of the genetically-modified eukaryotic cell. In some embodiments, expression of the target gene is disrupted by insertion of the polynucleotide.

In some embodiments, the target gene is a TCR alpha gene. In some embodiments, the target gene is a TCR alpha constant region gene. In some embodiments, the target gene is a TCR beta gene. In some embodiments, the target gene is a TCR beta constant region gene. In some embodiments, wherein the target gene is a TCR alpha constant region gene, the nucleic acid sequence encoding the TGFB-1 inhibitory agent is positioned within a sequence set forth in SEQ ID NO: 20. In certain embodiments, the nucleic acid sequence encoding the TGFB-1 inhibitory agent is positioned between nucleotides 13 and 14 of SEQ ID NO: 20. In some embodiments, the genetically-modified eukaryotic cell does not have detectable cell surface expression of an endogenous alpha/beta TCR. In some embodiments, the genetically-modified eukaryotic cell does not have detectable cell surface expression of endogenous CD3.

In some embodiments, the target gene is a TGFBR2 gene. In some embodiments, the genetically-modified eukaryotic cell does not have detectable cell surface expression of TGFBR2.

In some embodiments, the nucleic acid sequence encoding the TGFB-1 inhibitory agent is operably linked to a promoter (e.g., a promoter described herein).

In certain embodiments, the engineered antigen receptor is a chimeric antigen receptor. In certain embodiments, the engineered antigen receptor is an exogenous T cell receptor. In certain embodiments, the engineered antigen receptor is a TCR fusion construct (TRuC).

In some embodiments, the nucleic acid sequence encoding the TGFB-1 inhibitory agent and the nucleic acid sequence encoding the engineered antigen receptor are positioned in different genes.

In some embodiments, the nucleic acid sequence encoding the TGFB-1 inhibitory agent and the nucleic acid sequence encoding the engineered antigen receptor are positioned in the same gene.

In some embodiments, the genetically-modified eukaryotic cell comprises a polynucleotide comprising the nucleic acid sequence encoding the TGFB-1 inhibitory agent and the nucleic acid sequence encoding the engineered antigen receptor.

In some embodiments, the polynucleotide comprises a promoter operably linked to the nucleic acid sequence encoding the TGFB-1 inhibitory agent and the nucleic acid sequence encoding the engineered antigen receptor.

In some embodiments, the nucleic acid sequence encoding the TGFB-1 inhibitory agent and the nucleic acid sequence encoding the engineered antigen receptor are separated by an IRES or 2A sequence.

In some embodiments, the polynucleotide comprises a first promoter operably linked to the nucleic acid sequence encoding the TGFB-1 inhibitory agent and a second promoter operably linked to the nucleic acid sequence encoding the engineered antigen receptor.

In some embodiments, the first promoter and the second promoter are identical.

In some embodiments, the first promoter and the second promoter are not identical.

In some embodiments, the first promoter and the second promoter are promoters described herein.

In another aspect, the invention provides a genetically-modified eukaryotic cell comprising in its genome: (a) an inactivated TGFB-1 gene, wherein expression of an encoded TGFB-1 protein is disrupted; and (b) a nucleic acid sequence encoding an engineered antigen receptor.

In certain embodiments, the engineered antigen receptor is a chimeric antigen receptor. In certain embodiments, the engineered antigen receptor is an exogenous T cell receptor. In certain embodiments, the engineered antigen receptor is a TCR fusion construct (TRuC).

In some embodiments, the inactivated TGFB-1 gene comprises an insertion, a deletion, or a substitution that disrupts expression of the TGFB-1 protein.

In some embodiments, the inactivated TGFB-1 gene comprises an exogenous polynucleotide that disrupts expression of the TGFB-1 protein.

In some embodiments, the insertion, deletion, or substitution is positioned within an engineered nuclease recognition sequence. In some embodiments, the engineered nuclease recognition sequence is an engineered meganuclease recognition sequence, a zinc finger nuclease recognition sequence, a TALEN recognition sequence, a compact TALEN recognition sequence, a CRISPR system nuclease recognition sequence, or a megaTAL recognition sequence. In some embodiments, the engineered nuclease recognition sequence is an engineered meganuclease recognition sequence.

In some embodiments, the exogenous polynucleotide comprises a nucleic acid sequence encoding a polypeptide of interest and/or a nucleic acid sequence encoding an inhibitory nucleic acid molecule.

In some embodiments, the polypeptide of interest is an engineered antigen receptor, or wherein the polypeptide of interest or the inhibitory nucleic acid molecule is a TGFB-1 inhibitory agent.

In some embodiments, the exogenous polynucleotide comprises a promoter operably linked to the nucleic acid sequence encoding the polypeptide of interest and/or the nucleic acid sequence encoding the inhibitory nucleic acid molecule. In some embodiments, the promoter is a promoter described herein.

In some embodiments, the genetically-modified eukaryotic cell comprises an inactived TCR alpha gene. In some embodiments, the genetically-modified eukaryotic cell comprises an inactivated TCR alpha constant region gene. In some embodiments, the genetically-modified eukaryotic cell comprises an inactivated TCR beta gene. In some embodiments, the genetically-modified eukaryotic cell comprises an inactivated TCR beta constant region gene. In some embodiments, the genetically-modified eukaryotic cell does not have detectable cell surface expression of an alpha/beta TCR. In some embodiments, the genetically-modified eukaryotic cell does not have detectable cell surface expression of CD3.

In some embodiments, the nucleic acid sequence encoding the engineered antigen receptor is positioned within a target gene. In some embodiments, expression of a polypeptide encoded by the target gene is disrupted.

In some embodiments, the target gene is a TCR alpha gene. In some embodiments, the target gene is a TCR alpha constant region gene. In some embodiments, the target gene is a TCR beta gene. In some embodiments, the target gene is a TCR beta constant region gene. In certain embodiments, the target gene is a TCR alpha constant region gene, and the nucleic acid sequence encoding the engineered antigen receptor is positioned within a sequence set forth in SEQ ID NO: 20. In particular embodiments, the nucleic acid sequence is positioned between nucleotides 13 and 14 of SEQ ID NO: 20.

In some embodiments, the nucleic acid sequence encoding the engineered antigen receptor is operably linked to a promoter. In some embodiments, the promoter is a promoter described herein.

In some embodiments of the aspects of a genetically-modified cell described above, the genetically-modified eukaryotic cell comprises in its genome a nucleic acid sequence encoding an HLA class I histocompatibility antigen, alpha chain E (HLA-E) fusion protein. In some such embodiments, the HLA-E fusion protein comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 22. In some such embodiments, the HLA-E fusion protein comprises an amino acid sequence set forth in SEQ ID NO: 22.

In some embodiments of the aspects of a genetically-modified cell described above, the genetically-modified eukaryotic cell comprises an inactivated TGFB receptor type II (TGFBR2) gene, wherein expression of an encoded TGFBR2 protein is disrupted. In some such embodiments, the genetically-modified eukaryotic cell does not have detectable cell-surface expression of TGFBR2.

In some such embodiments, the inactivated TGFBR2 gene comprises an insertion, a deletion, or a substitution that disrupts expression of the TGFBR2 protein. In some such embodiments, the insertion, deletion, or substitution is positioned within an engineered nuclease recognition sequence. In some such embodiments, the engineered nuclease recognition sequence is an engineered meganuclease recognition sequence, a zinc finger nuclease recognition sequence, a TALEN recognition sequence, a compact TALEN recognition sequence, a CRISPR system nuclease recognition sequence, or a megaTAL recognition sequence. In some such embodiments, the engineered nuclease recognition sequence is an engineered meganuclease recognition sequence.

In some such embodiments, the inactivated TGFBR2 gene comprises an exogenous polynucleotide that disrupts expression of the TGFBR2 protein.

In some such embodiments, the exogenous polynucleotide comprises a nucleic acid sequence encoding a polypeptide of interest and/or a nucleic acid sequence encoding an inhibitory nucleic acid molecule.

In some such embodiments, the polypeptide of interest is an engineered antigen receptor, or the polypeptide of interest or the inhibitory nucleic acid molecule is a TGFB-1 inhibitory agent.

In some such embodiments, the exogenous polynucleotide comprises a promoter operably linked to the nucleic acid sequence encoding the polypeptide of interest and/or the nucleic acid sequence encoding the inhibitory nucleic acid molecule. In some such embodiments, the promoter is a promoter disclosed herein.

In some such embodiments, the genetically-modified eukaryotic cell comprises an inactived TCR alpha gene. In some such embodiments, the genetically-modified eukaryotic cell comprises an inactivated TCR alpha constant region gene. In some such embodiments, the genetically-modified eukaryotic cell comprises an inactivated TCR beta gene. In some such embodiments, the genetically-modified eukaryotic cell comprises an inactivated TCR beta constant region gene. In some such embodiments, the genetically-modified eukaryotic cell does not have detectable cell surface expression of an alpha/beta TCR. In some such embodiments, the genetically-modified eukaryotic cell does not have detectable cell surface expression of CD3.

In some embodiments of the aspects of a genetically-modified cell described above, the genetically-modified eukaryotic cell comprises in its genome a nucleic acid sequence encoding a TGFBR2 inhibitory agent.

In some such embodiments, the TGFBR2 inhibitory agent reduces expression of TGFBR2 protein in the genetically-modified eukaryotic cell.

In some such embodiments, the TGFBR2 inhibitory agent is an inhibitory nucleic acid molecule. In some such embodiments, the inhibitory nucleic acid molecule is an RNA interference molecule. In some such embodiments, the RNA interference molecule is a short hairpin RNA (shRNA). In some such embodiments, the RNA interference molecule is a small interfering RNA (siRNA). In some such embodiments, the RNA interference molecule is a hairpin siRNA. In some such embodiments, the RNA interference molecule is microRNA (miRNA). In some such embodiments, the RNA interference molecule is a precursor miRNA.

In some such embodiments, the RNA interference molecule is a shRNAmiR. In some such embodiments, the shRNAmiR can be any shRNAmiR described herein, which comprises guide and passenger strands having specificity for the TGFBR2 message.

In some such embodiments, the TGFBR2 inhibitory agent is an engineered DNA-binding domain comprising a transcriptional repressor domain.

In some such embodiments, expression of the TGFBR2 protein by the genetically-modified eukaryotic cell is reduced by about 50% to about 99%, by about 55% to about 99%, by about 60% to about 99%, by about 65% to about 99%, by about 70% to about 99%, by about 75% to about 99%, by about 80% to about 99%, by about 85% to about 99%, by about 90% to about 99%, by about 95% to about 99%, by about 75% to about 95%, by about 80% to about 95%, by about 85% to about 95%, or by about 90% to about 95%, compared to a control cell.

In some such embodiments, expression of TGFBR2 protein by the genetically-modified eukaryotic cell is reduced by about 50%, by about 55%, by about 60%, by about 65%, by about 70%, by about 75%, by about 80%, by about 81%, by about 82%, by about 83%, by about 84%, by about 85%, by about 86%, by about 87%, by about 88%, by about 89%, by about 90%, by about 91%, by about 92%, by about 93%, by about 94%, by about 95%, by about 96%, by about 97%, by about 98%, or by about 99%, compared to a control cell.

In some such embodiments, the polynucleotide comprising the nucleic acid sequence encoding the TGFB-1 inhibitory agent comprises the nucleic acid sequence encoding the TGFBR2 inhibitory agent. In some such embodiments, the polynucleotide comprising the nucleic acid sequence encoding the TGFB-1 inhibitory agent and the engineered antigen receptor comprises the nucleic acid sequence encoding the TGFBR2 inhibitory agent.

In some embodiments of the aspects above describing a genetically-modified eukaryotic cell, the genetically-modified eukaryotic cell is a human cell. In some such embodiments, the genetically-modified eukaryotic cell is an immune cell. In some such embodiments, the immune cell is a T cell, or a cell derived therefrom. In some such embodiments, the immune cell is a natural killer (NK) cell, or a cell derived therefrom. In some such embodiments, the immune cell is a B cell, or a cell derived therefrom. In some such embodiments, the immune cell is a monocyte, or a cell derived therefrom. In some such embodiments, the immune cell is a macrophage, or a cell derived therefrom. In some such embodiments, the genetically-modified eukaryotic cell is a stem cell. In some such embodiments, the genetically-modified eukaryotic cell is an induced-pluripotent stem cell (iPSC).

In another aspect, the invention provides a method of producing a genetically-modified eukaryotic cell, the method comprising introducing a first template nucleic acid into a eukaryotic cell, wherein the first template nucleic acid is inserted into the genome of the eukaryotic cell, wherein the first template nucleic acid comprises a first polynucleotide comprising a nucleic acid sequence encoding a TGFB-1 inhibitory agent, wherein the TGFB-1 inhibitory agent is expressed by the genetically-modified eukaryotic cell.

In some embodiments, the first template nucleic acid is inserted into the genome of the eukaryotic cell by random integration.

In some embodiments, the first template nucleic acid is introduced into the eukaryotic cell using a recombinant lentivirus.

In some embodiments, the genetically-modified eukaryotic cell comprises an inactived TCR alpha gene. In some embodiments, the genetically-modified eukaryotic cell comprises an inactivated TCR alpha constant region gene. In some embodiments, the genetically-modified eukaryotic cell comprises an inactivated TCR beta gene. In some embodiments, the genetically-modified eukaryotic cell comprises an inactivated TCR beta constant region gene. In some embodiments, the genetically-modified eukaryotic cell does not have detectable cell surface expression of an alpha/beta TCR. In some embodiments, the genetically-modified eukaryotic cell does not have detectable cell surface expression of CD3.

In some embodiments, the target gene is a TCR alpha gene. In some embodiments, the target gene is a TCR alpha constant region gene. In some embodiments, the target gene is a TCR beta gene. In some embodiments, the target gene is a TCR beta constant region gene. In certain embodiments, the target gene is a TCR alpha constant region gene, and the nucleic acid sequence encoding the engineered antigen receptor is positioned within a sequence set forth in SEQ ID NO: 20. In particular embodiments, the nucleic acid sequence encoding the engineered antigen receptor is positioned between nucleotides 13 and 14 of SEQ ID NO: 20. In some embodiments, the genetically-modified eukaryotic cell does not have detectable cell surface expression of an alpha/beta TCR. In some embodiments, the genetically-modified eukaryotic cell does not have detectable cell surface expression of CD3.

In some embodiments, the method comprises introducing into the eukaryotic cell a second template nucleic acid, wherein the second template nucleic acid comprises a second polynucleotide comprising a nucleic acid sequence encoding an engineered antigen receptor, and wherein the second template nucleic acid is integrated into the genome of the eukaryotic cell.

In some embodiments, the second template nucleic acid is randomly integrated in the genome.

In some embodiments, the second template nucleic acid is introduced into the eukaryotic cell using a recombinant lentivirus.

In some embodiments, the genetically-modified eukaryotic cell comprises an inactived TCR alpha gene. In some embodiments, the genetically-modified eukaryotic cell comprises an inactivated TCR alpha constant region gene. In some embodiments, the genetically-modified eukaryotic cell comprises an inactivated TCR beta gene. In some embodiments, the genetically-modified eukaryotic cell comprises an inactivated TCR beta constant region gene. In some embodiments, the genetically-modified eukaryotic cell does not have detectable cell surface expression of an alpha/beta TCR. In some embodiments, the genetically-modified eukaryotic cell does not have detectable cell surface expression of CD3.

In some embodiments, the method comprises introducing into the eukaryotic cell an engineered nuclease, or a nucleic acid sequence encoding an engineered nuclease, having specificity for a recognition sequence in the genome, wherein the engineered nuclease is expressed by the genetically-modified cell and generates a cleavage site at the recognition sequence, and wherein the second template nucleic acid is inserted into the cleavage site.

In some embodiments, the second template nucleic acid is flanked by homology arms having homology to sequences flanking the cleavage site, and wherein the second template nucleic acid is inserted at the cleavage site by homologous recombination.

In some embodiments, the recognition sequence is within a target gene, and wherein expression of the target gene is disrupted by the second template nucleic acid.

In some embodiments, the target gene is a TCR alpha gene. In some embodiments, the target gene is a TCR alpha constant region gene. In some embodiments, the target gene is a TCR beta gene. In some embodiments, the target gene is a TCR beta constant region gene. In some embodiments, the target gene is a TCR alpha constant region gene, and the second template nucleic acid is inserted within SEQ ID NO: 20. In some embodiments, the second template nucleic acid is inserted between nucleotides 13 and 14 of SEQ ID NO: 20. In some embodiments, genetically-modified eukaryotic cell does not have detectable cell surface expression of an alpha/beta TCR. In some embodiments, the genetically-modified eukaryotic cell does not have detectable cell surface expression of CD3.

In some embodiments, the engineered nuclease is an engineered meganuclease, a zinc finger nuclease, a TALEN, a compact TALEN, a CRISPR system nuclease, or a megaTAL.

In some embodiments, the engineered nuclease is an engineered meganuclease.

In some embodiments, the second template nucleic acid is introduced into the eukaryotic cell by a recombinant virus. In some embodiments, the recombinant virus is a recombinant adeno-associated virus (AAV). In some embodiments, the recombinant AAV has a serotype of AAV6 or AAV2.

In some embodiments, the nucleic acid encoding the engineered nuclease is introduced into the eukaryotic cell using an mRNA. In some embodiments, the nucleic acid encoding the engineered nuclease is introduced into the eukaryotic cell by electroporation. In some embodiments, the nucleic acid encoding the engineered nuclease is introduced into the eukaryotic cell by a lipid nanoparticle.

In some embodiments, the engineered antigen receptor is a chimeric antigen receptor, an exogenous TCR, or a TRuC.

In another aspect, the invention provides a method of producing a genetically-modified eukaryotic cell, the method comprising introducing a first template nucleic acid into a eukaryotic cell, wherein the first template nucleic acid is inserted into the genome of the eukaryotic cell, wherein the first template nucleic acid comprises a first polynucleotide comprising a nucleic acid sequence encoding a TGFB-1 inhibitory agent, wherein the TGFB-1 inhibitory agent is expressed and reduces the expression of TGFB-1 protein produced by the genetically-modified eukaryotic cell.

In some embodiments, the method comprises introducing into the eukaryotic cell an engineered nuclease, or a nucleic acid encoding an engineered nuclease, having specificity for a recognition sequence in the genome, wherein the engineered nuclease is expressed in the genetically-modified eukaryotic cell and generates a cleavage site at the recognition sequence, and wherein the template nucleic acid is inserted into the genome of the eukaryotic cell at the cleavage site.

In some embodiments, the template nucleic acid is flanked by homology arms having homology to sequences flanking the cleavage site, and wherein the template nucleic acid is inserted at the cleavage site by homologous recombination.

In some embodiments, the template nucleic acid is introduced into the eukaryotic cell by a recombinant virus. In some embodiments, the recombinant virus is a recombinant adeno-associated virus (AAV). In some embodiments, the recombinant virus has a serotype of AAV6 or AAV2.

In some embodiments, the nucleic acid encoding the engineered nuclease is introduced into the eukaryotic cell using an mRNA. In some embodiments, the nucleic acid encoding the engineered nuclease is introduced into the eukaryotic cell by electroporation. In some embodiments, the nucleic acid encoding the engineered nuclease is introduced into the eukaryotic cell by a lipid nanoparticle.

In some embodiments, the engineered nuclease is an engineered meganuclease, a zinc finger nuclease, a TALEN, a compact TALEN, a CRISPR system nuclease, or a megaTAL. In some embodiments, the engineered nuclease is an engineered meganuclease.

In some embodiments, the first polynucleotide comprises a promoter that is operably linked to the nucleic acid sequence encoding the TGFB-1 inhibitory agent. In some embodiments, the promoter is a promoter described herein.

In some embodiments, the recognition sequence is within a target gene. In some embodiments, expression of the target gene is disrupted by insertion of the template nucleic acid.

In some embodiments, the target gene is a TCR alpha gene. In some embodiments, the target gene is a TCR alpha constant region gene. In some embodiments, the target gene is a TCR beta gene. In some embodiments, the target gene is a TCR beta constant region gene. In some embodiments, the target gene is a TCR alpha constant region gene, and the template nucleic acid is inserted within a sequence set forth in SEQ ID NO: 20. In some embodiments, the template nucleic acid is inserted between nucleotides 13 and 14 of SEQ ID NO: 20. In some embodiments, the genetically-modified eukaryotic cell does not have detectable cell surface expression of an endogenous alpha/beta TCR. In some embodiments, the genetically-modified eukaryotic cell does not have detectable cell surface expression of endogenous CD3.

In some embodiments, the target gene is a TGFBR2 gene. In some embodiments, the genetically-modified eukaryotic cell does not have detectable cell surface expression of cell surface TGFBR2.

In some embodiments, the first polynucleotide comprising the nucleic acid encoding the TGFB-1 inhibitory agent comprises a nucleic acid sequence encoding an engineered antigen receptor.

In some embodiments, the first polynucleotide comprises a promoter operably linked to the nucleic acid sequence encoding the TGFB-1 inhibitory agent and the nucleic acid sequence encoding the engineered antigen receptor. In some embodiments, the nucleic acid sequence encoding the TGFB-1 inhibitory agent and the nucleic acid sequence encoding the engineered antigen receptor are separated by an IRES or 2A sequence.

In some embodiments, the first polynucleotide comprises a first promoter operably linked to the nucleic acid sequence encoding the TGFB-1 inhibitory agent and a second promoter operably linked to the nucleic acid sequence encoding the engineered antigen receptor. In some embodiments, the first promoter and the second promoter are identical. In some embodiments, the first promoter and the second promoter are promoters described herein.

In some embodiments, the first promoter and the second promoter are not identical. In some embodiments, the first promoter and the second promoter are promoters described herein.

In some embodiments, the genetically-modified eukaryotic cell comprises in its genome a nucleic acid sequence encoding an engineered antigen receptor.

In some embodiments, the genetically-modified eukaryotic cell comprises an inactived TCR alpha gene, an inactivated TCR alpha constant region gene, an inactivated TCR beta gene, or an inactivated TCR beta constant region gene.

In some embodiments, the nucleic acid sequence encoding the engineered antigen receptor is randomly integrated in the genome.

In some embodiments, the genetically-modified eukaryotic cell does not have detectable cell surface expression of an alpha/beta TCR. In some embodiments, wherein the genetically-modified eukaryotic cell does not have detectable cell surface expression of CD3.

In some embodiments, the nucleic acid sequence encoding the engineered antigen receptor is positioned within a target gene, and wherein expression of the target gene is disrupted.

In some embodiments, the target gene is a TCR alpha gene. In some embodiments, the target gene is a TCR alpha constant region gene. In some embodiments, the target gene is a TCR beta gene. In some embodiments, the target gene is a TCR beta constant region gene. In some embodiments, the target gene is a TCR alpha constant region gene, and the nucleic acid sequence encoding the engineered antigen receptor is positioned within SEQ ID NO: 20. In some embodiments, the nucleic acid sequence encoding the engineered antigen receptor is positioned between nucleotides 13 and 14 of SEQ ID NO: 20. In some embodiments, the genetically-modified eukaryotic cell does not have detectable cell surface expression of an alpha/beta TCR. In some embodiments, the genetically-modified eukaryotic cell does not have detectable cell surface expression of CD3.

In some embodiments, the target gene is a TGFBR2 gene. In some embodiments, the genetically-modified eukaryotic cell does not have detectable cell surface expression of TGFBR2.

In some embodiments, the method comprises introducing into the eukaryotic cell a second template nucleic acid, wherein the second template nucleic acid comprises a second polynucleotide comprising a nucleic acid sequence encoding an engineered antigen receptor, and wherein the second template nucleic acid is integrated into the genome of the eukaryotic cell.

In some embodiments, the second template nucleic acid is randomly integrated in the genome. In some embodiments, the second template nucleic acid is introduced into the eukaryotic cell using a recombinant lentivirus.

In some embodiments, the genetically-modified eukaryotic cell comprises an inactived TCR alpha gene. In some embodiments, the genetically-modified eukaryotic cell comprises an inactivated TCR alpha constant region gene. In some embodiments, the genetically-modified eukaryotic cell comprises an inactivated TCR beta gene. In some embodiments, the genetically-modified eukaryotic cell comprises an inactivated TCR beta constant region gene. In some embodiments, the genetically-modified eukaryotic cell does not have detectable cell surface expression of an alpha/beta TCR. In some embodiments, the genetically-modified eukaryotic cell does not have detectable cell surface expression of CD3.

In some embodiments, the nucleic acid sequence encoding the engineered antigen receptor is located within a different gene as the template nucleic acid. In some embodiments, wherein the nucleic acid sequence encoding the engineered antigen receptor is located within the same gene as the template nucleic acid.

In some embodiments, the engineered antigen receptor is a chimeric antigen receptor. In some embodiments, the engineered antigen receptor is an exogenous T cell receptor. In some embodiments, the engineered antigen receptor is a TRuC.

In some embodiments, the method comprises introducing into the eukaryotic cell a second engineered nuclease, or a nucleic acid encoding a second engineered nuclease, having specificity for a second recognition sequence in the genome, wherein the second engineered nuclease is expressed by the genetically-modified cell and generates a second cleavage site at the second recognition sequence, and introducing into the eukaryotic cell a second template nucleic acid, wherein the second template nucleic acid comprises a second polynucleotide comprising a nucleic acid sequence encoding an engineered antigen receptor, wherein the second template nucleic acid is inserted into the second cleavage site.

In some embodiments, the second template nucleic acid is flanked by homology arms having homology to sequences flanking the cleavage site, and wherein the second template nucleic acid is inserted at the cleavage site by homologous recombination.

In some embodiments, the second recognition sequence is within a target gene, and expression of the target gene is disrupted by the second template nucleic acid.

In some embodiments, the target gene is a TCR alpha gene. In some embodiments, the target gene is a TCR alpha constant region gene. In some embodiments, the target gene is a TCR beta gene. In some embodiments, the target gene is a TCR beta constant region gene. In some embodiments, the target gene is a TCR alpha constant region gene, and the second template nucleic acid is inserted within SEQ ID NO: 20. In some embodiments, the second template nucleic acid is inserted between nucleotides 13 and 14 of SEQ ID NO: 20. In some embodiments, the genetically-modified eukaryotic cell does not have detectable cell surface expression of an alpha/beta TCR. In some embodiments, the genetically-modified eukaryotic cell does not have detectable cell surface expression of CD3.

In some embodiments, the second engineered nuclease is an engineered meganuclease, a zinc finger nuclease, a TALEN, a compact TALEN, a CRISPR system nuclease, or a megaTAL. In some embodiments, the second engineered nuclease is an engineered meganuclease.

In some embodiments, the second template nucleic acid is introduced into the eukaryotic cell by a recombinant virus. In some embodiments, the recombinant virus is a recombinant adeno-associated virus (AAV). In some embodiments, the recombinant virus has a serotype of AAV6. In some embodiments, the recombinant virus has a serotype of AAV2.

In some embodiments, the nucleic acid encoding the second engineered nuclease is introduced into the eukaryotic cell using an mRNA. In some embodiments, the nucleic acid encoding the second engineered nuclease is introduced into the eukaryotic cell by electroporation. In some embodiments, the nucleic acid encoding the second engineered nuclease is introduced into the eukaryotic cell by a lipid nanoparticle.

In some embodiments, the second polynucleotide comprises a promoter operably linked to the nucleic acid sequence encoding the engineered antigen receptor. In some embodiments, the promoter is a promoter described herein.

In some embodiments, the engineered antigen receptor is a chimeric antigen receptor. In some embodiments, the engineered antigen receptor is an exogenous T cell receptor. In some embodiments, the engineered antigen receptor is a TRuC.

In some embodiments, the TGFB-1 inhibitory agent reduces expression of TGFB-1 protein in the genetically-modified eukaryotic cell.

In some embodiments, the TGFB-1 inhibitory agent is an inhibitory nucleic acid molecule. In some embodiments, the inhibitory nucleic acid molecule is an RNA interference molecule.

In some embodiments, the TGFB-1 inhibitory agent is an inhibitory nucleic acid molecule. In certain embodiments, the inhibitory nucleic acid molecule is an RNA interference molecule. In some embodiments, the RNA interference molecule is a short hairpin RNA (shRNA). In some embodiments, the RNA interference molecule is a small interfering RNA (siRNA). In some embodiments, the RNA interference molecule is a hairpin siRNA. In some embodiments, the RNA interference molecule is microRNA (miRNA). In some embodiments, the RNA interference molecule is a precursor miRNA.

In some embodiments, the RNA interference molecule is an miRNA-adapted shRNA (shRNAmiR). In certain embodiments, the shRNAmiR comprises, from 5′ to 3′: (a) a 5′ miR scaffold domain; (b) a 5′ miR basal stem domain; (c) a passenger strand; (d) a miR loop domain; (e) a guide strand; (f) a 3′ miR basal stem domain; and (g) a 3′ miR scaffold domain. In certain embodiments, the miR loop domain is a miR-30a loop domain, a miR-15 loop domain, a miR-16 loop domain, a miR-155 loop domain, a miR-22 loop domain, a miR-103 loop domain, or a miR-107 loop domain. In particular embodiments, the miR loop domain is a miR-30a loop domain. In some embodiments, the miR-30a loop domain comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 3. In particular embodiments, the miR-30a loop domain comprises a nucleic acid sequence set forth in SEQ ID NO: 3. In some embodiments, the shRNAmiR comprises a microRNA-E (miR-E) scaffold, a miR-30 (e.g., miR-30a) scaffold, a miR-15 scaffold, a miR-16 scaffold, a miR-155 scaffold, a miR-22 scaffold, a miR-103 scaffold, or a miR-107 scaffold. In certain embodiments, the shRNAmiR comprises a miR-E scaffold. In some embodiments, the shRNAmiR comprises a structure wherein: (a) the 5′ miR scaffold domain comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 4; (b) the 5′ miR basal stem domain comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 5; (c) the 3′ miR basal stem domain comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 6; and/or (d) the 3′ miR scaffold domain comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 7. In particular embodiments, the shRNAmiR comprises a structure wherein: (a) the 5′ miR scaffold domain comprises a nucleic acid sequence set forth in SEQ ID NO: 4; (b) the 5′ miR basal stem domain comprises a nucleic acid sequence set forth in SEQ ID NO: 5; (c) the 3′ miR basal stem domain comprises a nucleic acid sequence set forth in SEQ ID NO: 6; and (d) the 3′ miR scaffold domain comprises a nucleic acid sequence set forth in SEQ ID NO: 7. In certain embodiments, the shRNAmiR comprises a guide strand comprising a nucleic acid sequence set forth in SEQ ID NO: 8 and a passenger strand comprising a nucleic acid sequence set forth in SEQ ID NO: 9. In certain embodiments, the shRNAmiR comprises a guide strand comprising a nucleic acid sequence set forth in SEQ ID NO: 10 and a passenger strand comprising a nucleic acid sequence set forth in SEQ ID NO: 11. In certain embodiments, the shRNAmiR comprises a guide strand comprising a nucleic acid sequence set forth in SEQ ID NO: 12 and a passenger strand comprising a nucleic acid sequence set forth in SEQ ID NO: 13. In certain embodiments, the shRNAmiR comprises a guide strand comprising a nucleic acid sequence set forth in SEQ ID NO: 14 and a passenger strand comprising a nucleic acid sequence set forth in SEQ ID NO: 15. In certain embodiments, the shRNAmiR comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 16. In certain embodiments, the shRNAmiR comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 17. In certain embodiments, the shRNAmiR comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 18. In certain embodiments, the shRNAmiR comprises a nucleic acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 19. In particular embodiments, the shRNAmiR comprises a nucleic acid sequence set forth SEQ ID NO: 16. In particular embodiments, the shRNAmiR comprises a nucleic acid sequence set forth SEQ ID NO: 17. In particular embodiments, the shRNAmiR comprises a nucleic acid sequence set forth SEQ ID NO: 18. In particular embodiments, the shRNAmiR comprises a nucleic acid sequence set forth SEQ ID NO: 19.

In some embodiments, the TGFB-1 inhibitory agent is an engineered DNA-binding domain comprising a transcriptional repressor domain.

In some embodiments, expression of the TGFB-1 protein by the genetically-modified eukaryotic cell is reduced by about 50% to about 99%, by about 55% to about 99%, by about 60% to about 99%, by about 65% to about 99%, by about 70% to about 99%, by about 75% to about 99%, by about 80% to about 99%, by about 85% to about 99%, by about 90% to about 99%, by about 95% to about 99%, by about 75% to about 95%, by about 80% to about 95%, by about 85% to about 95%, or by about 90% to about 95%, compared to a control cell.

In some embodiments, expression of TGFB-1 protein by the genetically-modified eukaryotic cell is reduced by about 50%, by about 55%, by about 60%, by about 65%, by about 70%, by about 75%, by about 80%, by about 81%, by about 82%, by about 83%, by about 84%, by about 85%, by about 86%, by about 87%, by about 88%, by about 89%, by about 90%, by about 91%, by about 92%, by about 93%, by about 94%, by about 95%, by about 96%, by about 97%, by about 98%, or by about 99%, compared to a control cell.

In some embodiments, the TGFB-1 inhibitory agent binds TGFB-1 protein produced by the genetically-modified eukaryotic cell. In some embodiments, the TGFB-1 inhibitory agent is an antibody, or antibody fragment, having specificity for the TGFB-1 protein. In some embodiments, the TGFB-1 inhibitory agent is a soluble TGFBR2 protein. In some embodiments, the TGFB-1 inhibitory agent is secreted by the genetically-modified eukaryotic cell.

In some embodiments of the method aspects described above, the genetically-modified eukaryotic cell comprises in its genome a nucleic acid sequence encoding an HLA-E fusion protein. In some such embodiments, the HLA-E fusion protein comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 22. In some such embodiments, the HLA-E fusion protein comprises an amino acid sequence set forth in SEQ ID NO: 22.

In some such embodiments, the first polynucleotide comprising the nucleic acid sequence encoding the TGFB-1 inhibitory agent comprises the nucleic acid sequence encoding the HLA-E fusion protein. In some such embodiments, the first polynucleotide comprising the nucleic acid sequence encoding the TGFB-1 inhibitory agent and the engineered antigen receptor comprises the nucleic acid sequence encoding the HLA-E fusion protein.

In some particular embodiments of the method aspects described above, the genetically-modified eukaryotic cell comprises in its genome: (a) a nucleic acid sequence encoding a TGFB-1 inhibitory agent; and (b) a nucleic acid sequence encoding an engineered antigen receptor (e.g., a chimeric antigen receptor or exogenous TCR); wherein the nucleic acid sequence encoding the TGFB-1 inhibitory agent and the nucleic acid sequence encoding the engineered antigen receptor are positioned within a TCR alpha constant region gene and disrupt expression of the TCR alpha constant region gene, and wherein the TGFB-1 inhibitory agent is a shRNAmiR described herein.

In some particular embodiments of the method aspects described above, the genetically-modified eukaryotic cell comprises in its genome: (a) an inactivated TGFB-1 gene; and (b) a nucleic acid sequence encoding an engineered antigen receptor (e.g., a chimeric antigen receptor or exogenous TCR); wherein the nucleic acid sequence encoding the engineered antigen receptor is positioned within a TCR alpha constant region gene and disrupts expression of the TCR alpha constant region gene.

In some particular embodiments of the method aspects described above, the genetically-modified eukaryotic cell comprises in its genome: (a) a nucleic acid sequence encoding a TGFB-1 inhibitory agent; (b) a nucleic acid sequence encoding an engineered antigen receptor (e.g., a chimeric antigen receptor or exogenous TCR); and (c) a nucleic acid sequence encoding a TGFBR2 inhibitory agent; wherein the nucleic acid sequence encoding the TGFB-1 inhibitory agent, the nucleic acid sequence encoding the engineered antigen receptor, and the nucleic acid sequence encoding the TGFBR2 inhibitory agent are positioned within a TCR alpha constant region gene and disrupt expression of the TCR alpha constant region gene, and wherein the TGFB-1 inhibitory agent and the TGFBR2 inhibitory agent are shRNAmiRs described herein.

In some particular embodiments of the method aspects described above, the genetically-modified eukaryotic cell comprises in its genome: (a) a nucleic acid sequence encoding a TGFB-1 inhibitory agent; (b) a nucleic acid sequence encoding an engineered antigen receptor (e.g., a chimeric antigen receptor or exogenous TCR); and (c) an inactivated TGFBR2 gene; wherein the nucleic acid sequence encoding the TGFB-1 inhibitory agent and the nucleic acid sequence encoding the engineered antigen receptor are positioned within a TCR alpha constant region gene and disrupt expression of the TCR alpha constant region gene, and wherein the TGFB-1 inhibitory agent is a shRNAmiR described herein.

In some particular embodiments of the method aspects described above, the genetically-modified eukaryotic cell comprises in its genome: (a) an inactivated TGFB-1 gene; (b) a nucleic acid sequence encoding an engineered antigen receptor (e.g., a chimeric antigen receptor or exogenous TCR); and (c) a nucleic acid sequence encoding a TGFBR2 inhibitory agent; wherein the nucleic acid sequence encoding the chimeric antigen receptor and the nucleic acid sequence encoding a TGFBR2 inhibitory agent are positioned within a TCR alpha constant region gene and disrupt expression of the TCR alpha constant region gene, and wherein the TGFBR2 inhibitory agent is a shRNAmiR described herein.

In some particular embodiments of the method aspects described above, the genetically-modified eukaryotic cell comprises in its genome: (a) an inactivated TGFB-1 gene; (b) a nucleic acid sequence encoding an engineered antigen receptor (e.g., a chimeric antigen receptor or exogenous TCR); and (c) in inactivated TGFBR2 gene; wherein the nucleic acid sequence encoding the chimeric antigen receptor is positioned within a TCR alpha constant region gene and disrupts expression of the TCR alpha constant region gene.

In another aspect, the invention provides a method of producing a genetically-modified eukaryotic cell, the method comprising introducing into a eukaryotic cell a first engineered nuclease, or a nucleic acid encoding a first engineered nuclease, having specificity for a first recognition sequence in the TGFB-1 gene, wherein the first engineered nuclease is expressed by the eukaryotic cell and generates a first cleavage site at the recognition sequence.

In some embodiments, the first engineered nuclease generates a substitution or an insertion that disrupts expression of TGFB-1 protein.

In some embodiments, the first cleavage site is repaired by non-homologous end joining, resulting in an insertion or deletion that disrupts expression of TGFB-1 protein.

In some embodiments, the method comprises introducing into the eukaryotic cell a first template nucleic acid comprising a first polynucleotide, wherein the first template nucleic acid is inserted into the genome of the eukaryotic cell at the first cleavage site in the TGFB-1 gene, and wherein insertion of the first template nucleic acid disrupts expression of TGFB-1 protein.

In some embodiments, the nucleic acid encoding the first engineered nuclease is introduced into the eukaryotic cell using an mRNA. In some embodiments, the nucleic acid encoding the first engineered nuclease is introduced into the eukaryotic cell by electroporation. In some embodiments, the nucleic acid encoding the first engineered nuclease is introduced into the eukaryotic cell by a lipid nanoparticle.

In some embodiments, the first engineered nuclease is an engineered meganuclease, a zinc finger nuclease, a TALEN, a compact TALEN, a CRISPR system nuclease, or a megaTAL. In some embodiments, the first engineered nuclease is an engineered meganuclease.

In some embodiments, the first template nucleic acid is flanked by homology arms having homology to sequences flanking the first cleavage site in the TGFB-1 gene, and wherein the first template nucleic acid is inserted at the first cleavage site by homologous recombination.

In some embodiments, the first template nucleic acid is introduced into the eukaryotic cell using a recombinant virus. In some embodiments, the recombinant virus is a recombinant AAV. In some embodiments, the recombinant AAV has a serotype of AAV6 or AAV2.

In some embodiments, the first polynucleotide comprises a nucleic acid sequence encoding a polypeptide of interest or an inhibitory nucleic acid.

In some embodiments, the genetically-modified eukaryotic cell comprises an inactived TCR alpha gene. In some embodiments, the genetically-modified eukaryotic cell comprises an inactivated TCR alpha constant region gene. In some embodiments, the genetically-modified eukaryotic cell comprises an inactivated TCR beta gene. In some embodiments, the genetically-modified eukaryotic cell comprises an inactivated TCR beta constant region gene. In some embodiments, the genetically-modified eukaryotic cell does not have detectable cell surface expression of an alpha/beta TCR. In some embodiments, the genetically-modified eukaryotic cell does not have detectable cell surface expression of CD3.

In some embodiments, the nucleic acid sequence encoding the engineered antigen receptor is positioned within a TCR alpha gene. In some embodiments, the nucleic acid sequence encoding the engineered antigen receptor is positioned within a TCR alpha constant region gene. In some embodiments, the nucleic acid sequence encoding the engineered antigen receptor is positioned within a TCR beta gene. In some embodiments, the nucleic acid sequence encoding the engineered antigen receptor is positioned within a TCR beta constant region gene. In some embodiments, the nucleic acid sequence encoding the engineered antigen receptor is positioned within a TCR alpha constant region gene and is positioned within a sequence set forth in SEQ ID NO: 20. In some embodiments, the nucleic acid sequence encoding the engineered antigen receptor is positioned between nucleotides 13 and 14 of SEQ ID NO: 20. In some embodiments, the genetically-modified eukaryotic cell does not have detectable cell surface expression of an alpha/beta TCR. In some embodiments, the genetically-modified eukaryotic cell does not have detectable cell surface expression of CD3.

In some embodiments, the polypeptide of interest encoded by the first polynucleotide is an engineered antigen receptor.

In some embodiments, the method comprises introducing into the eukaryotic cell a second template nucleic acid comprising a second polynucleotide comprising a nucleic acid sequence encoding an engineered antigen receptor, wherein the second template nucleic acid is inserted into the genome of the eukaryotic cell.

In some embodiments, the second template nucleic acid is inserted into the genome of the eukaryotic cell by random integration. In some embodiments, the second template nucleic acid is introduced into the eukaryotic cell using a recombinant lentivirus.

In some embodiments, the genetically-modified eukaryotic cell comprises an inactived TCR alpha gene. In some embodiments, the genetically-modified eukaryotic cell comprises an inactivated TCR alpha constant region gene. In some embodiments, the genetically-modified eukaryotic cell comprises an inactivated TCR beta gene. In some embodiments, the genetically-modified eukaryotic cell comprises an inactivated TCR beta constant region gene. In some embodiments, the genetically-modified eukaryotic cell does not have detectable cell surface expression of an alpha/beta TCR. In some embodiments, the genetically-modified eukaryotic cell does not have detectable cell surface expression of CD3.

In some embodiments, the method comprises introducing into the eukaryotic cell a second engineered nuclease, or a nucleic acid encoding a second engineered nuclease, wherein the second engineered nuclease has specificity for a second recognition sequence in the genome of the eukaryotic cell, wherein the second engineered nuclease is expressed in the eukaryotic cell and generates a second cleavage site at the second recognition sequence, and wherein the second template nucleic acid is inserted into the genome of the eukaryotic cell at the second cleavage site.

In some embodiments, the second template nucleic acid is flanked by homology arms having homology to sequences flanking the second cleavage site, and wherein the second template nucleic acid is inserted at the second cleavage site by homologous recombination.

In some embodiments, the second template nucleic acid is introduced into the eukaryotic cell using a recombinant virus. In some embodiments, the recombinant virus is a recombinant AAV. In some embodiments, the recombinant AAV has a serotype of AAV6. In some embodiments, the recombinant AAV has a serotype of AAV2.

In some embodiments, the nucleic acid encoding the second engineered nuclease is introduced into the eukaryotic cell using an mRNA. In some embodiments, the nucleic acid encoding the second engineered nuclease is introduced into the eukaryotic cell by electroporation. In some embodiments, the nucleic acid encoding the second engineered nuclease is introduced into the eukaryotic cell by a lipid nanoparticle.

In some embodiments, the second engineered nuclease is an engineered meganuclease, a zinc finger nuclease, a TALEN, a compact TALEN, a CRISPR system nuclease, or a megaTAL. In some embodiments, the second engineered nuclease is an engineered meganuclease.

In some embodiments, the second recognition sequence is within a target gene. In some embodiments, the target gene is disrupted by insertion of the second template nucleic acid.

In some embodiments, the target gene is a TCR alpha gene. In some embodiments, the target gene is a TCR alpha constant region gene. In some embodiments, the target gene is a TCR beta gene. In some embodiments, the target gene is a TCR beta constant region gene. In some embodiments, the target gene is a TCR alpha constant region gene, and the second template nucleic acid is inserted within a sequence set forth in SEQ ID NO: 20. In some embodiments, the second template nucleic acid is inserted between nucleotides 13 and 14 of SEQ ID NO: 20. In some embodiments, the engineered antigen receptor is a chimeric antigen receptor. In some embodiments, the engineered antigen receptor is an exogenous T cell receptor. In some embodiments, the engineered antigen receptor is a TRuC.

In some embodiments of the method aspects described above, the genetically-modified eukaryotic cell comprises in its genome a nucleic acid sequence encoding an HLA-E fusion protein. In some such embodiments, the HLA-E fusion protein comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to a sequence set forth in SEQ ID NO: 22. In some such embodiments, the HLA-E fusion protein comprises an amino acid sequence set forth in SEQ ID NO: 22. In some such embodiments, the second polynucleotide comprises the nucleic acid sequence encoding the HLA-E fusion protein.

In some embodiments of the method aspects described above, the genetically-modified eukaryotic cell comprises an inactivated TGFBR2 gene, wherein expression of TGFBR2 protein is disrupted. In some such embodiments, the inactivated TGFBR2 gene comprises an insertion, deletion, or substitution that disrupts expression of the TGFBR2 protein.

In some such embodiments, the inactivated TGFBR2 gene comprises an exogenous polynucleotide that disrupts expression of the TGFBR2 protein. In some such embodiments, the exogenous polynucleotide comprises a nucleic acid sequence encoding a polypeptide of interest and/or a nucleic acid sequence encoding an inhibitory nucleic acid molecule. In some such embodiments, the polypeptide of interest is an engineered antigen receptor, or wherein the polypeptide of interest or the inhibitory nucleic acid molecule is a TGFB-1 inhibitory agent.

In some such embodiments, the exogenous polynucleotide comprises a promoter operably linked to the nucleic acid sequence encoding the polypeptide of interest and/or the nucleic acid sequence encoding the inhibitory nucleic acid molecule. In some such embodiments, the promoter is a promoter described herein.

In some such embodiments, the genetically-modified eukaryotic cell does not have detectable cell-surface expression of TGFBR2.

In some such embodiments, the insertion, deletion, or substitution is positioned within an engineered nuclease recognition sequence. In some such embodiments, the engineered nuclease recognition sequence is an engineered meganuclease recognition sequence, a zinc finger nuclease recognition sequence, a TALEN recognition sequence, a compact TALEN recognition sequence, a CRISPR system nuclease recognition sequence, or a megaTAL recognition sequence. In some such embodiments, the engineered nuclease recognition sequence is an engineered meganuclease recognition sequence.

In some such embodiments, the method comprises introducing into the eukaryotic cell an engineered nuclease, or a nucleic acid encoding an engineered nuclease, having specificity for a recognition sequence within the TGFBR2 gene, wherein the engineered nuclease is expressed in the eukaryotic cell and generates a cleavage site at the recognition sequence to produce a modified TGFBR2 gene.

In some such embodiments, the engineered nuclease generates a substitution or an insertion at the cleavage site that disrupts expression of the encoded TGFBR2 protein.

In some such embodiments, the cleavage site is repaired by non-homologous end joining, and wherein the modified TGFBR2 gene comprises an insertion or deletion at the cleavage site that disrupts expression of the encoded TGFBR2 protein.

In some such embodiments, the method comprises introducing into the eukaryotic cell a template nucleic acid comprising a polynucleotide, wherein the template nucleic acid is inserted into the genome of the eukaryotic cell at the cleavage site in the TGFBR2 gene, and wherein insertion of the template nucleic acid disrupts expression of the encoded TGFBR2 protein.

In some such embodiments, the template nucleic acid is flanked by homology arms having homology to sequences flanking the cleavage site in the TGFBR2 gene, and wherein the template nucleic acid is inserted at the cleavage site by homologous recombination.

In some such embodiments, the polynucleotide comprises a nucleic acid sequence encoding a polypeptide of interest and/or a nucleic acid sequence encoding an inhibitory nucleic acid molecule. In some such embodiments, the polypeptide of interest is an engineered antigen receptor, or the polypeptide of interest or the inhibitory nucleic acid molecule is a TGFB-1 inhibitory agent.

In some such embodiments, the polynucleotide comprises a promoter operably linked to the nucleic acid sequence encoding the polypeptide of interest and/or the nucleic acid sequence encoding the inhibitory nucleic acid molecule. In some such embodiments, the promoter is a promoter described herein.

In some such embodiments, the genetically-modified eukaryotic cell does not have detectable cell-surface expression of TGFBR2.

In some such embodiments, the template nucleic acid is introduced into the eukaryotic cell using a recombinant virus. In some such embodiments, the recombinant virus is a recombinant AAV. In some such embodiments, the recombinant AAV has a serotype of AAV6. In some such embodiments, the recombinant AAV has a serotype of AAV6.

In some such embodiments, the nucleic acid encoding the engineered nuclease is introduced into the eukaryotic cell using an mRNA. In some such embodiments, the nucleic acid encoding the engineered nuclease is introduced into the eukaryotic cell by electroporation. In some such embodiments, the nucleic acid encoding the engineered nuclease is introduced into the eukaryotic cell by a lipid nanoparticle.

In some such embodiments, the engineered nuclease is an engineered meganuclease, a zinc finger nuclease, a TALEN, a compact TALEN, a CRISPR system nuclease, or a megaTAL. In some such embodiments, the engineered nuclease is an engineered meganuclease.

In some embodiments of the method aspects described above, the genetically-modified eukaryotic cell comprises in its genome a nucleic acid sequence encoding a TGFBR2 inhibitory agent.

In some such embodiments, the method comprises introducing into the eukaryotic cell a template nucleic acid comprising a polynucleotide comprising a nucleic acid sequence encoding a TGFBR2 inhibitory agent, wherein the template nucleic acid is inserted into the genome of the eukaryotic cell, and wherein the TGFBR2 inhibitory agent is expressed.

In some such embodiments, the template nucleic acid is inserted into the genome of the eukaryotic cell by random integration. In some such embodiments, the template nucleic acid is introduced into the eukaryotic cell using a recombinant lentivirus.

In some such embodiments, the genetically-modified eukaryotic cell comprises an inactived TCR alpha gene. In some such embodiments, the genetically-modified eukaryotic cell comprises an inactivated TCR alpha constant region gene. In some such embodiments, the genetically-modified eukaryotic cell comprises an inactivated TCR beta gene. In some such embodiments, the genetically-modified eukaryotic cell comprises an inactivated TCR beta constant region gene. In some such embodiments, the genetically-modified eukaryotic cell does not have detectable cell surface expression of an alpha/beta TCR. In some such embodiments, the genetically-modified eukaryotic cell does not have detectable cell surface expression of CD3.

In some such embodiments, the method comprises introducing into the eukaryotic cell an engineered nuclease, or a nucleic acid encoding an engineered nuclease, wherein the engineered nuclease has specificity for a recognition sequence in the genome of the eukaryotic cell, wherein the engineered nuclease is expressed in the eukaryotic cell and generates a cleavage site at the recognition sequence, and wherein the template nucleic acid is inserted into the genome of the eukaryotic cell at the cleavage site.

In some such embodiments, the template nucleic acid is flanked by homology arms having homology to sequences flanking the cleavage site, and wherein the template nucleic acid is inserted at the cleavage site by homologous recombination.

In some such embodiments, the template nucleic acid is introduced into the eukaryotic cell using a recombinant virus. In some such embodiments, the recombinant virus is a recombinant AAV. In some such embodiments, the recombinant AAV has a serotype of AAV6. In some such embodiments, the recombinant AAV has a serotype of AAV2.

In some such embodiments, the nucleic acid encoding the engineered nuclease is introduced into the eukaryotic cell using an mRNA. In some such embodiments, the nucleic acid encoding the engineered nuclease is introduced into the eukaryotic cell by electroporation. In some such embodiments, the nucleic acid encoding the engineered nuclease is introduced into the eukaryotic cell by a lipid nanoparticle.

In some such embodiments, the engineered nuclease is an engineered meganuclease, a zinc finger nuclease, a TALEN, a compact TALEN, a CRISPR system nuclease, or a megaTAL. In some such embodiments, the engineered nuclease is an engineered meganuclease.

In some such embodiments, the recognition sequence is within a target gene. In some such embodiments, expression of the target gene is disrupted by insertion of the donor template nucleic acid.

In some such embodiments, the target gene is a TCR alpha gene. In some such embodiments, the target gene is a TCR alpha constant region gene. In some such embodiments, the target gene is a TCR beta gene. In some such embodiments, the target gene is a TCR beta constant region gene. In some such embodiments, the target gene is a TCR alpha constant region gene, and the template nucleic acid is inserted within a sequence set forth in SEQ ID NO: 20. In some such embodiments, the template nucleic acid is inserted between nucleotides 13 and 14 of SEQ ID NO: 20. In some such embodiments, the genetically-modified eukaryotic cell does not have detectable cell surface expression of an endogenous alpha/beta TCR. In some such embodiments, the genetically-modified eukaryotic cell does not have detectable cell surface expression of endogenous CD3.

In some such embodiments, the TGFBR2 inhibitory agent reduces expression of TGFBR2 protein in the genetically-modified eukaryotic cell.

In some such embodiments, the TGFBR2 inhibitory agent is an inhibitory nucleic acid molecule.

In some such embodiments, the inhibitory nucleic acid molecule is an RNA interference molecule. In some such embodiments, the RNA interference molecule is a short hairpin RNA (shRNA), a small interfering RNA (siRNA), a hairpin siRNA, a microRNA (miRNA), a precursor miRNA, or an miRNA-adapted shRNA (shRNAmiR). In some such embodiments, the shRNAmiR is a shRNAmiR described herein which is adapted to comprise guide and passenger strands having specificity for the TGFBR2 message.

In some such embodiments, the TGFBR2 inhibitory agent is an engineered DNA-binding domain comprising a transcriptional repressor domain.

In some such embodiments, expression of TGFBR2 protein by the genetically-modified eukaryotic cell is reduced by about 50% to about 99%, by about 55% to about 99%, by about 60% to about 99%, by about 65% to about 99%, by about 70% to about 99%, by about 75% to about 99%, by about 80% to about 99%, by about 85% to about 99%, by about 90% to about 99%, by about 95% to about 99%, by about 75% to about 95%, by about 80% to about 95%, by about 85% to about 95%, or by about 90% to about 95%, compared to a control cell.

In some such embodiments, expression of TGFBR2 protein by the genetically-modified eukaryotic cell is reduced by about 50%, by about 55%, by about 60%, by about 65%, by about 70%, by about 75%, by about 80%, by about 81%, by about 82%, by about 83%, by about 84%, by about 85%, by about 86%, by about 87%, by about 88%, by about 89%, by about 90%, by about 91%, by about 92%, by about 93%, by about 94%, by about 95%, by about 96%, by about 97%, by about 98%, or by about 99%, compared to a control cell.

In some such embodiments, the polynucleotide comprising the nucleic acid sequence encoding the TGFB-1 inhibitory agent comprises the nucleic acid sequence encoding the TGFBR2 inhibitory agent.

In some such embodiments, the polynucleotide comprising the nucleic acid sequence encoding the TGFB-1 inhibitory agent and the engineered antigen receptor comprises the nucleic acid sequence encoding the TGFBR2 inhibitory agent.

In some embodiments of the method aspects described above, the genetically-modified eukaryotic cell is a human cell. In some embodiments, the genetically-modified eukaryotic cell is an immune cell. In some such embodiments, the immune cell is a T cell, or a cell derived therefrom. In some such embodiments, the immune cell is a natural killer (NK) cell, or a cell derived therefrom. In some such embodiments, the immune cell is a B cell, or a cell derived therefrom. In some such embodiments, the immune cell is a monocyte, or a cell derived therefrom. In some such embodiments, the immune cell is a macrophage, or a cell derived therefrom. In some such embodiments, the genetically-modified eukaryotic cell is a stem cell. In some such embodiments, the genetically-modified eukaryotic cell is an iPSC.

In another aspect, the invention provides a genetically-modified eukaryotic cell prepared by any of the methods described herein.

In another aspect, the invention provides a population of cells comprising a plurality of any genetically-modified eukaryotic cell described herein.

In another aspect, the invention provides a pharmaceutical composition comprising any genetically-modified eukaryotic cell described herein.

In another aspect, the invention provides a pharmaceutical composition comprising a population of cells described herein.

In another aspect, the invention provides a method for reducing the number of target cells in a subject in need thereof, the method comprising administering to the subject an effective amount of a population of cells described herein, or a pharmaceutical composition described herein, wherein the genetically-modified eukaryotic cells express an engineered antigen receptor having specificity for an antigen present on the target cells.

In some embodiments, the method is a method of immunotherapy.

In some embodiments, the target cells are cancer cells.

In some embodiments, the method reduces the size of the cancer.

In some embodiments, the method eradicates the cancer in the subject.

In another aspect, the invention provides a population of genetically-modified eukaryotic cells described herein, or a pharmaceutical composition described herein, for use as a medicament. In another aspect, the invention provides a population of genetically-modified eukaryotic cells described herein, or a pharmaceutical composition described herein, for use in the manufacture of a medicament. In some such aspects, the medicament is useful for reducing the number of target cells in a subject in need thereof. In some such embodiments, the target cells are cancer cells. In some such aspects, the medicament is useful for cancer immunotherapy in subjects in need thereof.

### BRIEF DESCRIPTIONS OF THE FIGURES

FIG. 1 provides a bar graph showing the percent (%) knockdown of TGFβ1 with various designed shRNAmiR constructs specific to TGFβ1 (72173-72176) or a control construct (72155).

FIGS. 2A and 2B. FIG. 2A provides a bar graph showing the number of CAR T cells following co-culture with K562 cells engineered to express BCMA (KBCMA, “Targets”) or KBCMA cells further engineered to secrete active TGFβ1 (“TGFβ targets”). The horizontal dashed line in FIG. 2A represents 20,000 cells. FIG. 2B provides a bargraph showing the percentage of cytolysis (target cell killing) of target cells co-cultured with either the KBCMA target cells or the KBCMA target cells expressing TGFβ1. Asterisks in each graph show the statistical significant difference in either CAR T cell number (FIG. 2A) or percent cytolysis (FIG. 2B) between the two co-culture conditions (*P<0.05, **P<0.01).

### BRIEF DESCRIPTIONS OF THE SEQUENCES

SEQ ID NO: 1 sets forth the amino acid sequence of a wild-type I-CreI meganuclease from Chlamydomonas reinhardtii.

SEQ ID NO: 2 sets forth the amino acid sequence of a LAGLIDADG domain.

SEQ ID NO: 3 sets forth the nucleic acid sequence of a miR-30a loop domain.

SEQ ID NO: 4 sets forth the nucleic acid sequence of a 5′ miR-E scaffold domain.

SEQ ID NO: 5 sets forth the nucleic acid sequence of a 5′ mir-E basal stem domain.

SEQ ID NO: 6 sets forth the nucleic acid sequence of a 3′ mir-E basal stem domain.

SEQ ID NO: 7 sets forth the nucleic acid sequence of a 3′ miR-E scaffold domain.

SEQ ID NO: 8 sets forth the nucleic acid sequence of a 72173 shRNAmiR guide strand.

SEQ ID NO: 9 sets forth the nucleic acid sequence of a 72173 shRNAmiR passenger strand.

SEQ ID NO: 10 sets forth the nucleic acid sequence of a 72174 shRNAmiR guide strand.

SEQ ID NO: 11 sets forth the nucleic acid sequence of a 72174 shRNAmiR passenger strand.

SEQ ID NO: 12 sets forth the nucleic acid sequence of a 72175 shRNAmiR guide strand.

SEQ ID NO: 13 sets forth the nucleic acid sequence of a 72175 shRNAmiR passenger strand.

SEQ ID NO: 14 sets forth the nucleic acid sequence of a 72176 shRNAmiR guide strand.

SEQ ID NO: 15 sets forth the nucleic acid sequence of a 72176 shRNAmiR passenger strand.

SEQ ID NO: 16 sets forth the nucleic acid sequence of a 72173 shRNAmiR.

SEQ ID NO: 17 sets forth the nucleic acid sequence of a 72174 shRNAmiR.

SEQ ID NO: 18 sets forth the nucleic acid sequence of a 72175 shRNAmiR.

SEQ ID NO: 19 sets forth the nucleic acid sequence of a 72176 shRNAmiR.

SEQ ID NO: 20 sets forth the nucleic acid sequence of a TRC 1-2 recognition sequence (sense).

SEQ ID NO: 21 sets forth the nucleic acid sequence of a TRC 1-2 recognition sequence (antisense).

SEQ ID NO: 22 sets forth the amino acid sequence of a an HLA-E fusion protein.

SEQ ID NO: 23 sets forth the nucleic acid sequence of a JeT promoter.

SEQ ID NO: 24 sets forth the nucleic acid sequence of an EF1 alpha core promoter.

SEQ ID NO: 25 sets forth the amino acid sequence of a TRC 1-2L.1592 meganuclease.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein, the terms “nuclease” and “endonuclease” are used interchangeably to refer to naturally-occurring or engineered enzymes, which cleave a phosphodiester bond within a polynucleotide chain. Engineered nucleases can include, without limitation, engineered meganucleases, zinc finger nucleases, TALENs, compact TALENs, CRISPR system nucleases, and megaTALs. In addition, any engineered nuclease is envisioned that is capable of generating overhangs at its cleavage site.

As used herein, the terms “cleave” or “cleavage” refer to the hydrolysis of phosphodiester bonds within the backbone of a recognition sequence within a target sequence that results in a double-stranded break within the target sequence, referred to herein as a “cleavage site”.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be an endonuclease that is derived from I-CreI (SEQ ID NO: 1), and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in the targeted cells as described herein such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit-Linker-C-terminal subunit. The two meganuclease subunits will generally be non-identical in amino acid sequence and will bind non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “linker” refers to an exogenous peptide sequence used to join two nuclease subunits into a single polypeptide. A linker may have a sequence that is found in natural proteins or may be an artificial sequence that is not found in any natural protein. A linker may be flexible and lacking in secondary structure or may have a propensity to form a specific three-dimensional structure under physiological conditions. A linker can include, without limitation, those encompassed by U.S. Pat. Nos. 8,445,251, 9,340,777, 9,434,931, and 10,041,053, each of which is incorporated by reference in its entirety. In some embodiments, a linker may have at least 80%, at least 85%, at least 90%, at least 91%, at least 92%, at least 93%, at least 94%, at least 95%, at least 96%, at least 97%, at least 98%, at least 99%, or 100% sequence identity to residues 154-195 of SEQ ID NO: 25.

As used herein, the term “compact TALEN” refers to an endonuclease comprising a DNA-binding domain with one or more TAL domain repeats fused in any orientation to any portion of the I-TevI homing endonuclease or any of the endonucleases listed in Table 2 in U.S. Application No. 20130117869 (which is incorporated by reference in its entirety), including but not limited to MmeI, EndA, End1, I-BasI, I-TevII, I-TevIII, I-TwoI, MspI, MvaI, NucA, and NucM. Compact TALENs do not require dimerization for DNA processing activity, alleviating the need for dual target sites with intervening DNA spacers. In some embodiments, the compact TALEN comprises 16-22 TAL domain repeats.

As used herein, the terms “CRISPR nuclease” or “CRISPR system nuclease” refers to a CRISPR (clustered regularly interspaced short palindromic repeats)-associated (Cas) endonuclease or a variant thereof, such as Cas9, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, type V enzyme, such as Cpf1. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a D10A mutation within the RuvC I catalytic domain, or with a H840A, N854A, or N863A mutation. CRISPR system nucleases can further include base editing systems, that allow for substitution of a single base at a target site, prime editing systems that allow for the substitution of segments of a target sequence in the genome, and CRISPR integrase-based systems that allow for the insertion of exogenous sequences into a targeted site. Given a predetermined DNA locus, recognition sequences can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407).

As used herein, the term “megaTAL” refers to a single-chain endonuclease comprising a transcription activator-like effector (TALE) DNA binding domain with an engineered, sequence-specific homing endonuclease.

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, S1 nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type IIs restriction endonuclease, including but not limited to FokI, FoM, StsI, HhaI, HindIII, Nod, BbvCI, EcoRI, BglI, and AlwI. Additional Type IIs restriction endonucleases are described in International Publication No. WO 2007/014275, which is incorporated by reference in its entirety. In some embodiments, the nuclease domain of the TALEN is a FokI nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12th and 13th amino acids. These two positions, referred to as the repeat variable dipeptide (RVD), are highly variable and show a strong correlation with specific nucleotide recognition. Each base pair in the DNA target sequence is contacted by a single TAL repeat with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The term “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein that binds to a pre-determined DNA sequence (see, for example, Boch et al. (2009) Science 326(5959):1509-1512 and Moscou and Bogdanove (2009) Science 326(5959):1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., FokI) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site.

As used herein, the terms “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, S1 nuclease, mung bean nuclease, pancreatic DNAse I, micrococcal nuclease, and yeast HO endonuclease. Nuclease domains useful for the design of zinc finger nucleases include those from a Type IIs restriction endonuclease, including but not limited to FokI, FoM, and StsI restriction enzyme. Additional Type IIs restriction endonucleases are described in International Publication No. WO 2007/014275, which is incorporated by reference in its entirety. The structure of a zinc finger domain is stabilized through coordination of a zinc ion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence ˜18 basepairs in length, comprising a pair of nine basepair half-sites separated by 2-10 basepairs. See, for example, U.S. Pat. Nos. 5,789,538, 5,925,523, 6,007,988, 6,013,453, 6,200,759, and International Publication Nos. WO 95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as FokI nuclease, it is possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four basepairs). It is understood that the term “zinc finger nuclease” can refer to a single zinc finger protein or, alternatively, a pair of zinc finger proteins (i.e., a left ZFN protein and a right ZFN protein) that bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell J G, Barbas C F 3rd. Zinc Finger Tools: custom DNA-binding domains for transcription factors and nucleases. Nucleic Acids Res. 2006 Jul. 1; 34 (Web Server issue):W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the terms “recombinant” or “engineered,” with respect to a protein, means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered.

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein, the term with respect to recombinant proteins, the term “modification” means any insertion, deletion, or substitution of an amino acid residue in the recombinant sequence relative to a reference sequence (e.g., a wild-type or a native sequence).

As used herein, the terms “recognition sequence” or “recognition site” refers to a DNA sequence that is bound and cleaved by a nuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a compact TALEN, the recognition sequence comprises a first CNNNGN sequence that is recognized by the I-TevI domain, followed by a non-specific spacer 4-16 basepairs in length, followed by a second sequence 16-22 bp in length that is recognized by the TAL-effector domain (this sequence typically has a 5′ T base). Cleavage by a compact TALEN produces two basepair 3′ overhangs. In the case of a CRISPR nuclease, the recognition sequence is the sequence, typically 16-24 basepairs, to which the guide RNA binds to direct cleavage. Full complementarity between the guide sequence and the recognition sequence is not necessarily required to effect cleavage. Cleavage by a CRISPR nuclease can produce blunt ends (such as by a class 2, type II CRISPR nuclease) or overhanging ends (such as by a class 2, type V CRISPR nuclease), depending on the CRISPR nuclease. In those embodiments wherein a CpfI CRISPR nuclease is utilized, cleavage by the CRISPR complex comprising the same will result in 5′ overhangs and in certain embodiments, 5 nucleotide 5′ overhangs. Each CRISPR nuclease enzyme also requires the recognition of a PAM (protospacer adjacent motif) sequence that is near the recognition sequence complementary to the guide RNA. The precise sequence, length requirements for the PAM, and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

As used herein, the terms “target site” or “target sequence” refers to a region of the chromosomal DNA of a cell comprising a recognition sequence for a nuclease.

As used herein, the terms “DNA-binding affinity” or “binding affinity” means the tendency of a nuclease to non-covalently associate with a reference DNA molecule (e.g., a recognition sequence or an arbitrary sequence). Binding affinity is measured by a dissociation constant, Kd. As used herein, a nuclease has “altered” binding affinity if the Kd of the nuclease for a reference recognition sequence is increased or decreased by a statistically significant percent change relative to a reference nuclease.

As used herein, the term “specificity” means the ability of a nuclease to bind and cleave double-stranded DNA molecules only at a particular sequence of base pairs referred to as the recognition sequence, or only at a particular set of recognition sequences. The set of recognition sequences will share certain conserved positions or sequence motifs but may be degenerate at one or more positions. A highly-specific nuclease is capable of cleaving only one or a very few recognition sequences. Specificity can be determined by any method known in the art.

As used herein, the term “homologous recombination” or “HR” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell.

As used herein, the term “non-homologous end-joining” or “NHEJ” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end-joining is error-prone and frequently results in the untemplated addition or deletion of DNA sequences at the site of repair. In some instances, cleavage at a target recognition sequence results in NHEJ at a target recognition site. Nuclease-induced cleavage of a target site in the coding sequence of a gene followed by DNA repair by NHEJ can introduce mutations into the coding sequence, such as frameshift mutations, that disrupt gene function. Thus, engineered nucleases can be used to effectively knock-out a gene in a population of cells.

As used herein, the term “disrupted” or “disrupts” or “disrupts expression” or “disrupting a target sequence” refers to the introduction of a mutation (e.g., frameshift mutation) that interferes with the gene function and prevents expression and/or function of the polypeptide/expression product encoded thereby. For example, nuclease-mediated disruption of a gene can result in the expression of a truncated protein and/or expression of a protein that does not retain its wild-type function. Additionally, introduction of a template nucleic acid into a gene can result in no expression of an encoded protein, expression of a truncated protein, and/or expression of a protein that does not retain its wild-type function.

As used herein, the term “homology arms” or “sequences homologous to sequences flanking a nuclease cleavage site” refer to sequences flanking the 5′ and 3′ ends of a polynucleotide, which promote insertion of the polynucleotide into a cleavage site generated by a nuclease. In general, homology arms can have a length of at least 50 base pairs, preferably at least 100 base pairs, and up to 2000 base pairs or more, and can have at least 90%, preferably at least 95%, or more, sequence homology to their corresponding sequences in the genome. In some embodiments, the homology arms are about 500 base pairs.

As used herein, the term with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:33 89-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

As used herein, the term “corresponding to” with respect to modifications of two proteins or amino acid sequences is used to indicate that a specified modification in the first protein is a substitution of the same amino acid residue as in the modification in the second protein, and that the amino acid position of the modification in the first protein corresponds to or aligns with the amino acid position of the modification in the second protein when the two proteins are subjected to standard sequence alignments (e.g., using the BLASTp program). Thus, the modification of residue “X” to amino acid “A” in the first protein will correspond to the modification of residue “Y” to amino acid “A” in the second protein if residues X and Y correspond to each other in a sequence alignment and despite the fact that X and Y may be different numbers.

As used herein, the term “recognition half-site,” “recognition sequence half-site,” or simply “half-site” means a nucleic acid sequence in a double-stranded DNA molecule that is recognized and bound by a monomer of a homodimeric or heterodimeric meganuclease or by one subunit of a single-chain meganuclease or by one subunit of a single-chain meganuclease.

As used herein, the term “hypervariable region” refers to a localized sequence within a meganuclease monomer or subunit that comprises amino acids with relatively high variability. A hypervariable region can comprise about 50-60 contiguous residues, about 53-57 contiguous residues, or preferably about 56 residues. In some embodiments, the residues of a hypervariable region may correspond to positions 24-79 or positions 215-270 of any one of SEQ ID NO: 25. A hypervariable region can comprise one or more residues that contact DNA bases in a recognition sequence and can be modified to alter base preference of the monomer or subunit. A hypervariable region can also comprise one or more residues that bind to the DNA backbone when the meganuclease associates with a double-stranded DNA recognition sequence. Such residues can be modified to alter the binding affinity of the meganuclease for the DNA backbone and the target recognition sequence. In different embodiments of the invention, a hypervariable region may comprise between 1-20 residues that exhibit variability and can be modified to influence base preference and/or DNA-binding affinity. In particular embodiments, a hypervariable region comprises between about 15-20 residues that exhibit variability and can be modified to influence base preference and/or DNA-binding affinity. In some embodiments, variable residues within a hypervariable region correspond to one or more of positions 24, 26, 28, 30, 32, 33, 38, 40, 42, 44, 46, 68, 70, 75, and 77 of SEQ ID NO: 25. In other embodiments, variable residues within a hypervariable region correspond to one or more of positions 215, 217, 219, 221, 223, 224, 229, 231, 233, 235, 237, 259, 261, 266, and 268 of any one of SEQ ID NO: 25.

As used herein, the term “reference level” in the context of protein or mRNA levels refers to a level of protein or mRNA as measured in, for example, a control cell, control cell population or a control subject, at a previous time point in the control cell, the control cell population or the subject undergoing treatment (e.g., a pre-dose baseline level obtained from the control cell, control cell population or subject), or a pre-defined threshold level of protein or mRNA (e.g., a threshold level identified through previous experimentation).

As used herein, the term “a control” or “a control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); or, (c) a cell genetically identical to the genetically-modified cell but which is not exposed to conditions or stimuli or further genetic modifications that would induce expression of altered genotype or phenotype.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, the term “vector” or “recombinant DNA vector” may be a construct that includes a replication system and sequences that are capable of transcription and translation of a polypeptide-encoding sequence in a given host cell. If a vector is used, then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention. In some embodiments, a “vector” also refers to a viral vector. Viral vectors can include, without limitation, retroviral vectors, lentiviral vectors, adenoviral vectors, and adeno-associated viral vectors (AAV).

As used herein, the term “operably linked” is intended to mean a functional linkage between two or more elements. For example, an operable linkage between a nucleic acid sequence encoding a nuclease as disclosed herein and a regulatory sequence (e.g., a promoter) is a functional link that allows for expression of the nucleic acid sequence encoding the nuclease. Operably linked elements may be contiguous or non-contiguous. When used to refer to the joining of two protein coding regions, by operably linked is intended that the coding regions are in the same reading frame.

As used herein, the term “inhibitory agent” is intended to include any agent which inhibits either the expression or function of a TGFβ family signaling molecule (e.g., TGFβ1, TGFβR1, or TGFβR2). For example, the inhibitory agent may be an inhibitory nucleic acid molecule such as RNA interference, which inhibits the mRNA of a TGFβ family signaling molecule thereby reducing its overall expression. Alternatively, the inhibitory agent may inhibit the expression of a TGFβ family signaling molecule at the transcriptional level by directly repressing gene expression through the use of engineered DNA binding proteins coupled with transcriptional repressor domains (e.g., an engineered zinc finger protein or engineered meganuclease coupled to a KRAB repressor domain). The inhibitory agent may inhibit the function of a TGFβ family signaling molecule by binding or sequestering the molecule. For example, an antibody, an antibody fragment, or a soluble TGFβ receptor can be used to bind to the TGFβ1 ligand. In the case of the TGFβ receptor, a receptor trap may be used to bind to, for example, TGFβR2 and prevent dimerization with and phosphorylation of TGFβR1.

As used herein, the term “RNA interference” or “RNAi” refers to a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559, which is incorporated by reference herein for its specific teachings thereof.

The term “siRNA” as used herein refers to small interfering RNA, also known as short interfering RNA or silencing RNA. siRNAs can be, for example, 18 to 30, 20 to 25, 21 to 23 or 21 nucleotide-long double-stranded RNA molecules. An “shRNA” as used herein is a short hairpin RNA, which is a sequence of RNA that makes a tight hairpin turn that can also be used to silence gene expression via RNA interference. shRNA can by operably linked to the U6 promoter expression. The shRNA hairpin structure is cleaved by the cellular machinery into siRNA. shRNA disclosed herein can comprise a sequence complementary to at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, or 23 nucleotides of the mRNA a target protein.

As used herein, the term “shRNA” or “short hairpin RNA” refers to an artificial RNA molecule comprising a hairpin that can be used to silence gene expression via RNA interference.

As used herein, the term “miRNA” or “microRNA” or “miR” refers to mature microRNAs (miRNAs) that are endogenously encoded ˜22 nt long RNAs that post-transcriptionally reduce the expression of target genes. miRNAs are found in plants, animals, and some viruses and are generally expressed in a highly tissue- or developmental-stage-specific fashion.

A “stem-loop structure” refers to a nucleic acid having a secondary structure that includes a region of nucleotides which are known or predicted to form a double strand (stem portion) that is linked on one side by a region of predominantly single-stranded nucleotides (loop portion), In some cases, the loop may also be very short and thereby not be recognized by Dicer, leading to Dicer-independent shRNAs (comparable to the endogenous miR0431). The term “hairpin” is also used herein to refer to stem-loop structures. The actual primary sequence of nucleotides within the stern-loop structure is not critical to the practice of the description as long as the secondary structure is present. As is known in the art, the secondary structure does not require exact base-pairing. Thus, the stem may include one or more base mismatches. Alternatively, the base-pairing may be exact (i.e., not include any mismatches).

As used herein, the terms “shRNAmiR” and “microRNA-adapted shRNA” refer to shRNA sequences embedded within a microRNA scaffold. A shRNAmiR molecule mimics naturally-occurring pri-miRNA molecules in that they comprise a hairpin flanked by sequences necessary for efficient processing and can be processed by the Drosha enzyme into pre-miRNAs, exported into the cytoplasm, and cleaved by Dicer, after which the mature miRNA can enter the RISC. The microRNA scaffold can be derived from naturally-occurring microRNA, pre-miRNAs, or pri-miRNAs or variants thereof. In some embodiments, the shRNA sequences which the shRNAmiR is based upon is of a different length from miRNAs (which are 22 nucleotides long) and the miRNA scaffold must therefore be modified in order to accommodate the longer or shorter shRNA sequence length.

As used herein, the term “microRNA flanking sequences” refers to nucleotide sequences comprising microRNA processing elements. MicroRNA processing elements are the minimal nucleic acid sequences which contribute to the production of mature microRNA from primary microRNA or precursor microRNA. Often these elements are located within a 40 nucleotide sequence that flanks a microRNA stem-loop structure. In some instances, the microRNA processing elements are found within a stretch of nucleotide sequences of between 5 and 4,000 nucleotides in length that flank a microRNA stem-loop structure. MicroRNA flanking sequences used in the shRNAmiR molecules can be naturally-occurring sequences flanking naturally-occurring microRNA or can be variants thereof. MicroRNA flanking sequences include miR scaffold domains and miR basal stem domains.

shRNAmiR molecules used in the presently disclosed compositions and methods can comprise in the 5′ to 3′ direction: (a) a 5′ miR scaffold domain; (b) a 5′ miR basal stem domain; (c) a passenger strand; (d) a miR loop domain; (e) a guide strand; (f) a 3′ miR basal stem domain; and (g) a 3′ miR scaffold domain.

As used herein, the term “miR scaffold domain” as it relates to a shRNAmiR refers to a nucleotide sequence that can flank either the 5′ or 3′ end of a microRNA or shRNA in a shRNAmiR molecule and can be derived from a naturally-occurring microRNA flanking sequence or a variant thereof. In general, the miR basal stem domain sequence separates the shRNA sequence (passenger and guide strand, and miR loop domain) and the scaffold domains. The 5′ miR scaffold domain can comprise a restriction enzyme (e.g., type IIS restriction enzyme) recognition sequence at or near its 3′ end and the 3′ miR scaffold domain can comprise a restriction enzyme recognition sequence at or near its 5′ end, thus facilitating the insertion of a shRNA sequence. In some embodiments, the secondary structure of the miR scaffold domain is more important than the actual sequence thereof.

As used herein, the term “miR basal stem domain” as it relates to a shRNAmiR refers to sequences immediately flanking the passenger and guide strand sequences that comprise the base of the hairpin stem below the passenger:guide duplex. Thus, the 5′ and 3′ miR basal stem domains are complementary (fully or partially) in sequence to one another. In some embodiments, the 5′ and 3′ miR basal stem domains comprise sequences that when hybridized together, form two mismatch bubbles, each comprising one or two mismatched base pairs.

As used herein, the term “passenger strand” as it relates to a shRNAmiR refers to the sequence of the shRNAmiR, which is complementary (fully or partially) to the guide sequence.

As used herein, the term “guide strand” as it relates to a shRNAmiR refers to the sequence of the shRNAmiR that has complementarity (full or partial) with the target mRNA sequence for which a reduction in expression is desired.

As used herein, a “miR loop domain” as it relates to a shRNAmiR refers to the single-stranded loop sequence at one end of the passenger:guide duplex of the shRNAmiR. The miR loop domain can be derived from a naturally-occurring pre-microRNA loop sequence or a variant thereof.

As used herein, the term “antibody” refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

As used herein, the terms an “antibody fragment”, “antigen-binding fragment” or “antigen-binding portion” of an antibody refers to a molecule other than an intact antibody that comprises a portion of an intact antibody and that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv); single domain antibodies (sdAbs), and multispecific antibodies formed from antibody fragments.

As used herein, the term an “engineered antigen receptor” refers to an exogenous receptor introduced into a cell, such as a chimeric antigen receptor or exogenous T cell receptor, which induces an activating signal in the cell upon stimulation/binding to a ligand or antigen (e.g., a tumor-specific antigen).

As used herein, the term “chimeric antigen receptor” or “CAR” refers to an engineered receptor that confers or grafts specificity for an antigen onto an immune effector cell (e.g., a human T cell). A chimeric antigen receptor comprises at least an extracellular ligand-binding domain or moiety, a transmembrane domain, and an intracellular domain that comprises one or more signaling domains and/or co-stimulatory domains.

In some embodiments, the extracellular ligand-binding domain or moiety is an antibody, or antibody fragment. In this context, the term “antibody fragment” can refer to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CHi domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine, humanized, or fully human.

The extracellular ligand-binding domain of a chimeric antigen receptor can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a chimeric antigen receptor can also comprise a naturally-occurring ligand for an antigen of interest, or a fragment of a naturally-occurring ligand which retains the ability to bind the antigen of interest.

The intracellular stimulatory domain can include one or more cytoplasmic signaling domains that transmit an activation signal to the T cell following antigen binding. Such cytoplasmic signaling domains can include, without limitation, a CD3 zeta signaling domain.

The intracellular stimulatory domain can also include one or more intracellular co-stimulatory domains that transmit a proliferative and/or cell-survival signal after ligand binding. Such intracellular co-stimulatory domains can be any of those known in the art and can include, without limitation, those co-stimulatory domains disclosed in WO 2018/067697 including, for example, Novel 6. Further examples of co-stimulatory domains can include 4-1BB (CD137), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof.

A chimeric antigen receptor further includes additional structural elements, including a transmembrane domain that is attached to the extracellular ligand-binding domain via a hinge or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (α chain), p75 (β chain) or γ chain, subunit chain of Fc receptors (e.g., Fcy receptor III) or CD proteins such as the CD8 alpha chain. In certain examples, the transmembrane domain is a CD8 alpha domain. Alternatively, the transmembrane domain can be synthetic and can comprise predominantly hydrophobic residues such as leucine and valine.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcγRllla receptor or IgG1. In certain examples, the hinge region can be a CD8 alpha domain.

As used herein, the terms “exogenous T cell receptor” or “exogenous TCR” refer to a TCR whose sequence is introduced into the genome of a eukaryotic cell (e.g., an immune cell such as a T cell) that may or may not endogenously express the TCR. Expression of an exogenous TCR on a immune cell can confer specificity for a specific epitope or antigen (e.g., an epitope or antigen preferentially present on the surface of a cancer cell or other disease-causing cell or particle). Such exogenous T cell receptors can comprise alpha and beta chains or, alternatively, may comprise gamma and delta chains. Exogenous TCRs useful in the invention may have specificity to any antigen or epitope of interest.

As used herein, the term “HLA class I histocompatibility antigen, alpha chain E fusion protein” or “HLA-E fusion protein” refers to a protein comprising an HLA-E protein fused to at least one additional protein that enables expression of the HLA-E protein on the cell-surface. HLA-E proteins can include, for example, an HLA-E-01:01 or HLA-E-01:03 protein. An HLA-E fusion protein can comprise, for example, an HLA-E protein fused to a beta-2 microglobulin protein that enables expression of the HLA-E protein on the cell-surface. In further examples, the HLA-E fusion protein can comprise an HLA-E protein fused to both a beta-2 microglobulin protein and an additional protein that is loaded into the HLA-E protein for presentation such as, for example, an HLA-G leader peptide and others known in the art. The proteins of the HLA-E fusion protein can be fused by polypeptide linkers such as, for example, a linkers described in PCT/US2020/026571, which is incorporated by reference herein in its entirety.

As used herein, the term “reduced expression” in reference to a target protein (i.e., an endogenously expressed protein) refers to any reduction in the expression of the endogenous protein by a genetically-modified cell when compared to a control cell. The term reduced can also refer to a reduction in the percentage of cells in a population of cells that express wild-type levels of an endogenous protein targeted by a shRNAmiR of the disclosure when compared to a population of control cells. Such a reduction in the percentage of cells in a population that fully express the targeted endogenous protein may be up to 5%, 10%, 20%, 30%, 40%, 50%, 60%, 70%, 80%, 90%, 95%, 96%, 97%, 98%, 99%, or up to 100%. It is understood in the context of this disclosure that the term “reduced” encompasses a partial or incomplete knockdown of a target or endogenous protein, and is distinguished from a complete knockdown, such as that achieved by gene inactivation by a nuclease.

As used herein, the term “T cell receptor alpha gene” or “TCR alpha gene” refer to the locus in a T cell which encodes the T cell receptor alpha subunit. The T cell receptor alpha gene can refer to NCBI Gene ID number 6955, before or after rearrangement. Following rearrangement, the T cell receptor alpha gene comprises an endogenous promoter, rearranged V and J segments, the endogenous splice donor site, an intron, the endogenous splice acceptor site, and the T cell receptor alpha constant region locus, which comprises the subunit coding exons.

As used herein, the term “T cell receptor alpha constant region” or “TCR alpha constant region” refers to the coding sequence of the T cell receptor alpha gene. The TCR alpha constant region includes the wild-type sequence, and functional variants thereof, identified by NCBI Gene ID NO. 28755.

As used herein, the term “T cell receptor beta gene” or “TCR beta gene” refers to the locus in a T cell which encodes the T cell receptor beta subunit. The T cell receptor beta gene can refer to NCBI Gene ID number 6957.

As used herein, the term “immune cell” refers to any cell that is part of the immune system (innate and/or adaptive) and is of hematopoietic origin. Non-limiting examples of immune cells include lymphocytes, B cells, T cells, monocytes, macrophages, dendritic cells, granulocytes, megakaryocytes, monocytes, macrophages, natural killer cells, myeloid-derived suppressor cells, innate lymphoid cells, platelets, red blood cells, thymocytes, leukocytes, neutrophils, mast cells, eosinophils, basophils, and granulocytes.

As used herein, a “human T cell” or “T cell” or “isolated human T cell” refers to a T cell isolated from a donor, particularly a human donor. T cells, and cells derived therefrom, include isolated T cells that have not been passaged in culture, T cells that have been passaged and maintained under cell culture conditions without immortalization, and T cells that have been immortalized and can be maintained under cell culture conditions indefinitely.

As used herein, a “human NK cell” or “NK cell” refers to a NK cell isolated from a donor, particularly a human donor. NK cells, and cells derived therefrom, include isolated NK cells that have not been passaged in culture, NK cells that have been passaged and maintained under cell culture conditions without immortalization, and NK cells that have been immortalized and can be maintained under cell culture conditions indefinitely.

As used herein, a “human B cell” or “B cell” refers to a B cell isolated from a donor, particularly a human donor. B cells, and cells derived therefrom, include isolated T cells that have not been passaged in culture, B cells that have been passaged and maintained under cell culture conditions without immortalization, and B cells that have been immortalized and can be maintained under cell culture conditions indefinitely.

As used herein, the terms “treatment” or “treating a subject” refers to the administration of a genetically-modified eukaryotic cell (e.g., a genetically-modified immune cell) or population of genetically-modified eukaryotic cells (e.g., a population of genetically-modified immune cells) of the invention to a subject having a disease. For example, the subject can have a disease such as cancer, and treatment can represent immunotherapy for the treatment of the disease. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some aspects, a genetically-modified eukaryotic cells (e.g., a genetically-modified immune cell) or population of genetically-modified eukaryotic cells (e.g., a population of genetically-modified immune cell) described herein is administered during treatment in the form of a pharmaceutical composition of the invention.

The term “effective amount” or “therapeutically effective amount” refers to an amount sufficient to effect beneficial or desirable biological and/or clinical results. The therapeutically effective amount will vary depending on the formulation or composition used, the disease and its severity and the age, weight, physical condition and responsiveness of the subject to be treated. In specific embodiments, an effective amount of a genetically-modified immune cell or population of genetically-modified immune cells of the invention, or pharmaceutical compositions disclosed herein, reduces at least one symptom of a disease in a subject. In those embodiments wherein the disease is a cancer, an effective amount of the pharmaceutical compositions disclosed herein reduces the level of proliferation or metastasis of cancer, causes a partial or full response or remission of cancer, or reduces at least one symptom of cancer in a subject.

As used herein, the term “cancer” should be understood to encompass any neoplastic disease (whether invasive or metastatic) which is characterized by abnormal and uncontrolled cell division causing malignant growth or tumor.

As used herein, the term “carcinoma” refers to a malignant growth made up of epithelial cells.

As used herein, the term “leukemia” refers to malignancies of the hematopoietic organs/systems and is generally characterized by an abnormal proliferation and development of leukocytes and their precursors in the blood and bone marrow.

As used herein, the term “sarcoma” refers to a tumor which is made up of a substance like the embryonic connective tissue and is generally composed of closely packed cells embedded in a fibrillary, heterogeneous, or homogeneous substance.

As used herein, the term “melanoma” refers to a tumor arising from the melanocytic system of the skin and other organs.

As used herein, the term “lymphoma” refers to a group of blood cell tumors that develop from lymphocytes.

As used herein, the term “blastoma” refers to a type of cancer that is caused by malignancies in precursor cells or blasts (immature or embryonic tissue).

As used herein, the recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range.

Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

### 2.1 Principle of the Invention

The TGFβ family of ligands and receptors are important regulators of immune homeostasis. In particular, TGFβ1 is a well-characterized peripheral tolerance enforcer on T lymphocytes. In mouse studies, conditional deletion (restricted to the T cell compartment) of TGFβ1 or its receptor is sufficient to induce fulminant multiorgan autoimmune responses that are lethal by 3-4 weeks of life (Huter et al., Eur. J. Immunol. 38(7) (2008)). Measurement of TGFβ1 transcript levels indicate that it is highly abundant in T cells following activation and/or proliferation. Thus, it is possible that T cells utilize TGFβ1 production in an autocrine signaling pathway for the suppression of T cell activation. In terms of engineered cells, this could result in a suppression of anti-tumor responses. Recent reports have shown that deletion of TGFβ1 in activated CD4⁺ cells and Treg cells is able to reduce tumor development in mice. This finding suggests that certain T cells in certain in vivo tumor environments are a source of TGFβ1 that can inhibit T cell anti-tumor effects (Donkor et al., Oncoimmunology 1(2), 2012). However, until the present disclosure, it has been completely unknown whether autocrine TGFβ1 signaling could inhibit expansion of genetically modified immune cells (e.g., CAR T cells) and/or their anti-tumor efficacy. Accordingly, it is contemplated herein that reducing TGFβ1 production by genetically modified immune cells (e.g., CAR T cells) may enhance the performance and anti-tumor effects of adoptive cell therapies.

Thus, in some embodiments described herein, TGFβ1 is reduced in a genetically modified eukaryotic cell (e.g., a genetically-modified immune cell such as a CAR T cell). The reduction of TGFβ1 may be accomplished through knock down of TGFβ1 or genetic knock out of TGFβ1 in the genetically modified immune cell. In addition, reduction of TGFβ1 may also be accompanied by knock down or knock out of its endogenous receptor (e.g., the TGFβR2 or TGFβR1 receptor tyrosine kinases). It is further contemplated that knock down of TGFβ1 and its receptor (e.g., TGFβR2 or TGFβR1) may result in one or more of the properties including increased proliferation, increased anti-tumor effects, and persistence compared to a genetically modified immune cell not having knock down of TGFβ1 and/or its receptor. In addition, knocking down TGFβ1 and/or its receptor (e.g., utilizing an inhibitory nucleic acid molecule) in a genetically modified immune cell reduces the number of genetic modification steps, which may further increase the persistence of the genetically modified immune cells.

### 2.2 Inhibitory Agents for Reducing TGFβ Signaling

In some embodiments, the disclosure provides inhibitory agents for reducing the expression of one or more members of the transforming growth factor beta super family of ligands and receptors. In particular embodiments, the disclosure provides an inhibitory agent for reducing the expression of TGFβ1. In particular embodiments, the disclosure provides an inhibitory agent for reducing the expression of TGFβR1. In particular embodiments, the disclosure provides an inhibitory agent for reducing the expression of TGFβR2. In some embodiments, the inhibitory agent is an inhibitory nucleic acid molecule such as RNA interference. In some embodiments, the inhibitory agent is clustered regularly interspaced short palindromic repeats interference (CRISPRi; Lei et al., Cell 152(5) 2013, which is incorporated by reference herein in its entirety). In some embodiments, the inhibitory agent is an engineered DNA binding protein fused with a transcriptional repressor domain. Examples of such proteins include but are not limited to zinc finger proteins fused with transcriptional repressor domains (U.S. Pat. No. 6,534,261, which is incorporated by reference herein for its specific teachings thereof) and engineered meganucleases fused with transcriptional repressor domains (US Patent Application Publication No. US20110123509, which is incorporated by reference herein in its entirety).

In some embodiments, the inhibitory agent is a binder that is specific for a TGFβ family member. Useful non-limiting inhibitory binding agents that can bind TGFβ1 and sequester TGFβ1 include but are not limited to antibodies, antibody fragments, soluble TGFβ receptors (e.g., a soluble TGFβR2). Useful and non-limiting inhibitory agents that can bind and inhibit TGFβR1 or TGFβR2 include but are not limited to antibodies, antibody fragments and TGFβ receptor traps, such as those described in Qin et al., Oncotarget 7(52) (2016), which is incorporated by reference herein. In some embodiments, the TGFB-1 inhibitory agent is secreted by a genetically-modified eukaryotic cell. In some embodiments, the TGFβ1 inhibitory agent is a soluble TGFβR2.

In some embodiments, the disclosure provides inhibitory nucleic acid molecules for reducing expression of TGFβ1. In some further embodiments, the disclosure provides inhibitory nucleic acid molecules for reducing expression of a receptor of TGFβ1 (e.g., the TGFβR2 or TGFβR1 receptor tyrosine kinases). In some embodiments, the inhibitory nucleic acid molecule comprises RNA interference including but not limited to a short hairpin RNA (shRNA), a small interfering RNA (siRNA), a hairpin siRNA, a microRNA (miRNA), a precursor miRNA, or an miRNA-adapted shRNA (shRNAmiR).

In certain embodiments, the disclosure provides a polynucleotide comprising: (a) a nucleic acid sequence encoding an engineered antigen receptor; (b) a 5′ homology arm; and (c) a 3′ homology arm. In other embodiments, the disclosure provides a polynucleotide comprising: (a) a first a nucleic acid sequence encoding an engineered antigen receptor; (b) a second a nucleic acid sequence encoding an inhibitory agent (e.g., an inhibitory nucleic acid molecule such as a shRNAmiR); (c) a 5′ homology arm; and (d) a 3′ homology arm. The 5′ homology arm and the 3′ homology arm can be engineered at any suitable length to have homology to chromosomal regions flanking a nuclease recognition sequence in a gene of interest, which can be any desired gene in a target cell in which a suitable recognition sequence is present.

In particular embodiments, the first and second nucleic acid sequences can be in the same orientation. This orientation can be either 5′ to 3′ relative to the homology arms or, alternatively, 3′ to 5′. In either case, the first nucleic acid sequence may be 5′ to the second nucleic acid sequence, or the second nucleic acid sequence may be 5′ to the first nucleic acid sequence. In other embodiments, the first and second nucleic acid sequences can be in different orientations in the polynucleotide. For example, the first nucleic acid sequence may be oriented 5′ to 3′, whereas the second nucleic acid sequence may be oriented 3′ to 5′. Alternatively, the first nucleic acid sequence may be oriented 3′ to 5′ and the second nucleic acid sequence may be oriented 5′ to 3′. Particular nucleic acid sequences and their relative orientation is described in PCT International Application Publication WO2018208837, which is incorporated by reference herein in its entirety.

In some embodiments, the polynucleotide can comprise multiple copies of the second nucleic acid sequence. The copies of the nucleic acid sequence can be identical or vary from one another. In some cases, the copies can include a promoter, a coding sequence for the inhibitory nucleic acid molecule, and a sequence, such as a (cPPT/CTS) sequence, to terminate translation of the inhibitory nucleic acid molecule. The copies of the second nucleic acid sequence can be in tandem to one another in the polynucleotide, and can be in the same orientation, or in opposite orientations. Alternatively, the copies may not be in tandem, and can be in the same orientation, or in opposite orientations.

The nucleic acid sequences of the polynucleotide can include various promoters that drive expression of the engineered antigen receptor and/or the inhibitory agent. One example of a suitable promoter is the immediate early cytomegalovirus (CMV) promoter sequence. This promoter sequence is a strong constitutive promoter sequence capable of driving high levels of expression of any nucleic acid sequence operatively linked thereto. Another example of a suitable promoter is Elongation Growth Factor-1α (EF-1α) (SEQ ID NO: 24). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the present disclosure should not be limited to the use of constitutive promoters. Inducible promoters are also contemplated as part of the present disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Synthetic promoters are also contemplated as part of the present disclosure. For example, in particular embodiments, the promoter driving expression of the engineered antigen receptor is a JeT promoter, such as the JeT promoter of SEQ ID NO: 23. In some embodiments, the promoter driving expression of the engineered antigen receptor is an EF1 alpha promoter, such as the EF1 alpha promoter of SEQ ID NO: 23.

In some particular embodiments, the promoter(s) utilized in the invention have at least 80%, at least 85%, at least 90%, at least 91%, at least 92%, at least 93%, at least 94%, at least 95%, at least 96%, at least 97%, at least 98%, at least 99%, or 100% sequence identity to a sequence set forth in SEQ ID NO: 23 (i.e., a JeT promoter) or SEQ ID NO: 24 (i.e., an EF1 alpha core promoter).

In some embodiments, the promoters are selected based on the desired outcome. It is recognized that different applications can be enhanced by the use of different promoters in the polynucleotides to modulate the timing, location and/or level of expression of the polynucleotides disclosed herein. Such expression constructs may also contain, if desired, a promoter regulatory region (e.g., one conferring inducible, constitutive, environmentally- or developmentally-regulated, or cell- or tissue-specific/selective expression), a transcription initiation start site, a ribosome binding site, an RNA processing signal, a transcription termination site, and/or a polyadenylation signal.

Promoters particularly useful for driving expression of an RNA interference molecule are well known in the art and can include, without limitation, pol III promoters, such as U6 or H1.

The 5′ and 3′ homology arms of the polynucleotide have sequence homology to corresponding sequences 5′ upstream and 3′ downstream of the nuclease recognition sequence in the genome. The homology arms promote insertion of the polynucleotide into the cleavage site generated by the nuclease. In general, homology arms can have a length of at least 50 base pairs, preferably at least 100 base pairs, and up to 2000 base pairs or more, and can have at least 90%, preferably at least 95%, or more, sequence homology to their corresponding sequences in the genome.

In order to assess the expression of an engineered antigen receptor (e.g. a CAR or exogenous T cell receptor) in a genetically-modified cell, the polynucleotide of the invention can optionally comprise an epitope which can be used to detect the presence of the encoded cell surface protein. In some examples described herein, a CAR coding sequence may include a QBend10 epitope which allows for detection using an anti-CD34 antibody (see, WO2013/153391).

In other examples, a polynucleotide can also contain either a selectable marker gene or a reporter gene, or both, to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes and fluorescent marker genes.

Expression may also be assessed by determining protein expression of the polypeptide targeted by the inhibitory nucleic acid sequence. For example, expression of TGFβ1 or its receptors (e.g., TGFβR2 or TGFβR1) can be detected on the cell surface by a number of techniques known in the art. Expression can also be determined by positive or negative selection procedures which purify particular populations of cells expressing, or lacking expression, of the cell surface polypeptides.

Also provided herein are vectors comprising the polynucleotides of the present disclosure. In some embodiments, the polynucleotide is cloned into a vector including, but not limited to a plasmid, a phagemid, a phage derivative, an animal virus, or a cosmid. Vectors of particular interest include expression vectors, replication vectors, probe generation vectors, and sequencing vectors.

In other embodiments, polynucleotides of the invention are provided on viral vectors, such as retroviral vectors, lentiviral vectors, adenoviral vectors, and adeno-associated viral (AAV) vectors. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193). Where the nucleic acid of the invention is provided in a viral vector that promotes random integration into the genome, and does not require the presence of 5′ and 3′ homology arms for homologous recombination, the nucleic acid of the invention can be provided without 5′ and 3′ homology arms.

In some embodiments, the present invention provides genetically-modified eukaryotic cells (e.g., genetically modified immune cells) expressing a shRNAmiR molecule that reduces the abundance of an endogenous protein (e.g., TGFβ1, TGFβR1 or TGFβR2). Inhibitory shRNAmiR molecules useful in the present disclosure include those that have been described in PCT International Patent Application No. PCT/US2020/026571, which is incorporated by reference herein in its entirety.

The shRNAmiR molecule can comprise a microRNA scaffold in that the structure of the shRNAmiR molecule can mimic that of a naturally-occurring microRNA (or pri-miRNA or pre-miRNA) or a variant thereof. Sequences of microRNAs (and pri-miRNAs and pre-miRNAs) are known in the art. Non-limiting examples of suitable miR scaffolds for the presently disclosed shRNAmiRs include miR-E, miR-30 (e.g., miR-30a), miR-15, miR-16, miR-155, miR-22, miR-103, and miR-107. In particular embodiments, the shRNAmiR used in the presently disclosed compositions and methods comprises a mir-E scaffold. The mir-E scaffold is a synthetically-derived variant of miR-30a and its genesis is described in International Publication No. WO 2014/117050, which is incorporated by reference in its entirety.

The presently disclosed shRNAmiR molecules can comprise the following domains in the 5′ to 3′ direction: (a) a 5′ miR scaffold domain; (b) a 5′ miR basal stem domain; (c) a passenger strand; (d) a miR loop domain; (e) a guide strand; (f) a 3′ miR basal stem domain; and (g) a 3′ miR scaffold domain. The miR scaffold domains and basal stem domains flank the miRNA stem-loop and are referred to herein as microRNA flanking sequences that comprise the microRNA processing elements (the minimal nucleic acid sequences which contribute to the production of mature microRNA from primary microRNA or precursor microRNA). Often these elements are located within a 40 nucleotide sequence that flanks a microRNA stem-loop structure. In some instances, the microRNA processing elements are found within a stretch of nucleotide sequences of between 5 and 4,000 nucleotides in length that flank a microRNA stem-loop structure.

In some embodiments, the miRNA flanking sequences are about 3 to about 4,000 nt in length and can be present on either or both the 5′ and 3′ ends of the shRNAmiR molecule. In other embodiments, the minimal length of the microRNA flanking sequence of the shRNAmiR molecule is about 10, about 20, about 30, about 40, about 50, about 60, about 70, about 80, about 90, about 100, about 125, about 126, about 127, about 128, about 129, about 130, about 131, about 132, about 133, about 134, about 135, about 136, about 137, about 138, about 139, about 140, about 150, about 200, and any integer therein between. In other embodiments the maximal length of the microRNA flanking sequence of the shRNAmiR molecule is about 2,000, about 2,100, about 2,200, about 2,300, about 2,400, about 2,500, about 2,600, about 2,700, about 2,800, about 2,900, about 3,000, about 3,100, about 3,200, about 3,300, about 3,400, about 3,500, about 3,600, about 3,700, about 3,800, about 3,900, about 4,000, and any integer therein between.

The microRNA flanking sequences may be native microRNA flanking sequences or artificial microRNA flanking sequences. A native microRNA flanking sequence is a nucleotide sequence that is ordinarily comprised within naturally existing systems with microRNA sequences (i.e., these sequences are found within the genomic sequences surrounding the minimal microRNA hairpin in vivo). Artificial microRNA flanking sequences are nucleotides sequences that are not found to be flanking microRNA sequences in naturally existing systems. The artificial microRNA flanking sequences may be flanking sequences found naturally in the context of other microRNA sequences. Alternatively, they may be composed of minimal microRNA processing elements which are found within naturally occurring flanking sequences and inserted into other random nucleic acid sequences that do not naturally occur as flanking sequences or only partially occur as natural flanking sequences.

In some embodiments, the 5′ miR scaffold domain is about 10 to about 150 nucleotides in length, including but not limited to about 10, about 20, about 30, about 40, about 50, about 60, about 70, about 80, about 90, about 100, about 110, about 120, about 130, about 140, and about 150 nucleotides long. In some of these embodiments, the 5′ miR scaffold domain is about 111 nucleotides in length. The 5′ miR scaffold domain may comprise a 3′ sequence that is a recognition sequence for a type IIS restriction enzyme. In some of these embodiments, the 5′ miR scaffold domain comprises a XhoI recognition sequence on its 3′ end. In particular embodiments, the 5′ miR scaffold domain has at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to the sequence set forth as SEQ ID NO: 4. In certain embodiments, the 5′ miR scaffold domain has the sequence set forth as SEQ ID NO: 4.

The 5′ miR basal stem domain of the shRNAmiR can be about 5 to about 30 nucleotides in length in some embodiments, including but not limited to about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, and about 30 nucleotides long. In some of these embodiments, the 5′ miR basal stem domain is about 20 nucleotides in length. In particular embodiments, the 5′ miR basal stem domain has at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to the sequence set forth as SEQ ID NO: 5. In certain embodiments, the 5′ miR basal stem domain has the sequence set forth as SEQ ID NO: 5.

The shRNAmiR molecules of the presently disclosed compositions and methods comprise a stem-loop structure, wherein the stem is comprised of the hybridized passenger and guide strands and the loop is single-stranded. The miR loop domain can be derived from a naturally-occurring pre-microRNA or pri-microRNA loop sequence or a variant thereof. In some embodiments, the miR loop domain has the sequence of a loop domain from any one of miR-30 (e.g., miR-30a), miR-15, miR-16, miR-155, miR-22, miR-103, and miR-107. In particular embodiments, the shRNAmiR comprises a miR-30a loop domain, the sequence of which is set forth as SEQ ID NO: 3.

In certain embodiments, the miR loop domain is about 5 to about 30 nucleotides in length, including but not limited to about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, and about 30 nucleotides long. In some of these embodiments, the miR loop domain is about 15 nucleotides in length. In particular embodiments, the miR loop domain has at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to the sequence set forth as SEQ ID NO: 3. In certain embodiments, the miR loop domain has the sequence set forth as SEQ ID NO: 3.

The 3′ miR basal stem domain of the shRNAmiR can be about 5 to about 30 nucleotides in length in some embodiments, including but not limited to about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, and about 30 nucleotides long. In some of these embodiments, the 3′ miR basal stem domain is about 18 nucleotides in length. In particular embodiments, the 3′ miR basal stem domain has at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to the sequence set forth as SEQ ID NO: 6. In certain embodiments, the 3′ miR basal stem domain has the sequence set forth as SEQ ID NO: 6.

In some embodiments, the 3′ miR scaffold domain is about 50 to about 150 nucleotides in length, including but not limited to about 10, about 20, about 30, about 40, about 50, about 60, about 70, about 80, about 90, about 100, about 110, about 120, about 130, about 140, or about 150 nucleotides long. In some of these embodiments, the 3′ miR scaffold domain is about 116 nucleotides in length. In particular embodiments, the 3′ miR scaffold domain has at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to the sequence set forth as SEQ ID NO: 7. In certain embodiments, the 3′ miR scaffold domain has the sequence set forth as SEQ ID NO: 7.

The guide strand of the shRNAmiR is the sequence that targets the mRNA, leading to reduction in abundance of the protein encoded by the mRNA. After the guide strand binds to its target mRNA, RISC either degrades the target transcript and/or prevents the target transcript from being loaded into the ribosome for translation. The guide strand is of sufficient complementarity with the target mRNA in order to lead to reduced expression of the target mRNA. In some embodiments, the guide strand is at least about 50%, at least about 60%, at least about 70%, at least about 80%, at least about 90%, at least about 95%, at least about 97%, at least about 98%, at least about 99% or 100% complementary to the target mRNA sequence. In certain embodiments, the guide strand hybridizes with the target mRNA within a coding sequence. The guide strand can comprise 1, 2, 3, 4, 5, or more mismatching nucleotides with the target mRNA sequence. In other embodiments, the guide strand hybridizes with the target mRNA in a non-coding region, such as a 5′ or 3′ untranslated region (UTR). In some embodiments, the guide strand is about 15 to about 25 nucleotides in length, including but not limited to about 15, about 16, about 17, about 18, about 19, about 20, about 21, about 22, about 23, about 24, and about 25 nucleotides long. In some of these embodiments, the guide strand is about 22 nucleotides in length. In particular embodiments wherein the shRNA sequence from which the shRNAmiR is derived is less than 22 nucleotides in length, which is the length of most naturally-occurring microRNAs, an additional nucleotide is added to the shRNA sequence and in certain embodiments, this additional nucleotide is one that is complementary with the corresponding position within the target mRNA.

The passenger strand of the shRNAmiR is the sequence that is fully or partially complementary with the guide strand sequence. In some embodiments, the passenger strand is about 15 to about 25 nucleotides in length, including but not limited to about 15 to about 25 nucleotides in length, including but not limited to about 15, about 16, about 17, about 18, about 19, about 20, about 21, about 22, about 23, about 24, and about 25 nucleotides long. In some of these embodiments, the passenger strand is about 22 nucleotides in length. The passenger strand can be at least about 50%, at least about 60%, at least about 70%, at least about 80%, at least about 90%, at least about 95%, at least about 97%, at least about 98%, at least about 99% or 100% complementary to the guide strand sequence. The passenger strand can comprise 1, 2, 3, 4, 5, or more mismatching nucleotides with the guide strand. In certain embodiments, however, the guide:passenger strand duplex does not comprise any mismatching nucleotides. In general, guide/passenger strand sequences should be selected that do not form any secondary structures within themselves. Further, the use of guide/passenger strand sequences that target sites within an mRNA that comprise single-nucleotide polymorphisms should be avoided. Guide/passenger strand sequences that are specific for the target mRNA are preferred to avoid any off-target effects (i.e., reduction in expression of non-target mRNAs).

In order to aid in the selection of suitable shRNAmiR guide/passenger strands, or sequences for other shRNAmiR domains, any program known in the art that models the predicted secondary structure of a RNA molecule can be used, including but not limited to Mfold, RNAfold, and UNAFold. Any program known in the art that can predict the efficiency of a shRNA or miRNA guide/passenger sequence to target a particular mRNA can be used to select suitable guide/passenger strand sequences, including but not limited to those disclosed in Agarwal et al. (2015) eLife 4:e05005; and Knott et al. (2014) Mol Cell 56(6):796-807, each of which is incorporated herein in its entirety.

### 2.3 Genetically-Modified Eukaryotic Cells

The invention provides genetically-modified eukaryotic cells and populations thereof and methods for producing the same. In some embodiments, the genetically-modified eukaryotic cells of the presently disclosed compositions and methods are genetically-modified immune cells. In some embodiments, the genetically-modified immune cells are genetically-modified human immune cells. In some embodiments, the immune cells are T cells, or cells derived therefrom. In other embodiments, the immune cells are natural killer (NK) cells, or cells derived therefrom. In still other embodiments, the immune cells are B cells, or cells derived therefrom. In yet other embodiments, the immune cells are monocyte or macrophage cells or cells derived therefrom.

Immune cells (e.g., T cells) can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines, NK cell lines, B cell lines, monocyte cells lines, or macrophage cell lines available in the art may be used. In some embodiments of the present disclosure, immune cells (e.g., T cells) are obtained from a unit of blood collected from a subject using any number of techniques known to the skilled artisan. In one embodiment, cells from the circulating blood of an individual are obtained by apheresis. Immune cells of the invention can also be induced pluripotent stem cell (iPSC)-derived cells that have been differentiated into functional immune cells (e.g., T cells, NK cell, B cells).

In some embodiments, a polynucleotide comprising a nucleic acid sequence encoding a TGFβ1 inhibitory agent described herein is positioned within a target gene of a genetically-modified eukaryotic cell (e.g., a genetically-modified immune cell). In some embodiments, the target gene comprises a TCR alpha gene, a TCR alpha constant region gene, a TCR beta gene, or a TCR beta constant region gene. In some embodiments, the target gene comprises a TCR alpha gene, a TCR alpha constant region gene. In some embodiments, the target gene is a TGFβR2 gene. In some embodiments, the target gene is a TGFβR1 gene. In some embodiments, the target gene is a TGFβ1 gene. In some embodiments, the genetically-modified eukaryotic cell does not have detectable expression of a TCR alpha constant region gene.

In some embodiments, the genetically-modified eukaryotic cells (e.g., genetically-modified immune cells) of the presently disclosed compositions and methods comprise in the cells' genome a nucleic acid sequence encoding an inhibitory agent (e.g., a shRNAmiR), leading to the reduction of expression of a target protein (e.g., TGFβ1).

In some of those embodiments wherein the expression of an endogenous protein is reduced, the expression of the endogenous protein is reduced by at least about 10%, about 20%, about 30%, about 40%, about 50%, about 55%, about 60%, about 65%, about 70%, about 75%, about 80%, about 85%, about 90%, about 95%, or up to about 99% compared to a control cell (e.g., a cell not expressing an inhibitory agent such as a shRNAmiR). Any method known in the art can be used to determine the expression level of an endogenous protein, including but not limited to, ELISA, flow cytometry, Western blot, immunocytochemistry, and immunoprecipitation.

Expressing an inhibitory agent described herein by cells within a population can lead to a reduction in the percentage of cells in the population of cells that fully express the endogenous protein to which the inhibitory agent is targeted when compared to a population of control cells. Such a reduction may be up to 5%, 10%, 20%, 30%, 40%, 50%, 60%, 70%, 80%, 90%, 95%, 96%, 97%, 98%, 99%, or up to 100% of cells in the population.

A nucleic acid sequence encoding an inhibitory agent described herein can be present in the genome of the genetically-modified eukaryotic cell (e.g., a genetically-modified immune cell), for example, in a polynucleotide. Such polynucleotides can be inserted into the genome, for example, by introducing a template nucleic acid of the invention either by random integration (e.g., lentiviral transduction) or by targeted insertion into a selected site (e.g., by nuclease-mediated targeted insertion). Polynucleotides comprising the inhibitory agent-encoding sequence can include, for example, nucleic acid sequences encoding additional proteins, such as those described herein (e.g., engineered antigen receptors such as CARs and exogenous TCRs as well as fusion proteins), and may also include control sequences such as promoters and termination sequences. The nucleic acid sequence encoding the inhibitory agent can be positioned at any number of locations within the polynucleotide or template nucleic acid that allow for expression of the inhibitory agent. In some examples, a nucleic acid sequence encoding an inhibitory agent is positioned between the stop codon of another transgene (e.g., a nucleic acid sequence encoding an engineered antigen receptor such as a CAR, exogenous TCR, or a fusion protein) and a termination signal. In other examples, another transgene present in the polynucleotide or template nucleic acid (e.g., a nucleic acid sequence encoding an engineered antigen receptor such as a CAR, exogenous TCR, or a fusion protein) comprises an intron that is positioned within the transgene sequence. Here, “positioned” is intended to mean that the intron sequence is inserted into the transgene sequence, such that the resulting sequence comprises a 5′ portion of the transgene, the intron sequence, and a 3′ portion of the transgene. In some such examples, the nucleic acid sequence encoding the inhibitory agent can be positioned within such an intron. Here, “positioned” is intended to mean that the inhibitory agent-encoding sequence is inserted into the intron sequence, such that the resulting sequence comprises a 5′ portion of the intron sequence, the inhibitory agent-encoding sequence, and a 3′ portion of the intron sequence. In such cases, the inhibitory agent is expressed and the intron sequence is spliced out by the cell when the transgene is expressed. Introns that can be included in this manner can be naturally-occurring introns or, alternatively, synthetic introns.

In some embodiments, the genetically modified eukaryotic cells have a target gene genetically knocked out. In some embodiments the genetically modified eukaryotic cells of the disclosure have a target gene knocked out by introducing an engineered nuclease that binds and cleaves a recognition sequence within the target gene, which results in the genetic knock out of the target gene within the cell that expresses the engineered nuclease. In some embodiments, an engineered nuclease binds and cleaves a recognition sequence within a TGFβ1. In some embodiments, an engineered nuclease binds and cleaves a recognition sequence within a TGFβR1. In some embodiments, an engineered nuclease binds and cleaves a recognition sequence within a TGFβR2. In some embodiments, the genetically-modified eukaryotic cell does not have detectable expression of a TGFβ1 gene. In some embodiments, the genetically-modified eukaryotic cell does not have detectable expression of a TGFβR1 gene. In some embodiments, the genetically-modified eukaryotic cell does not have detectable expression of a TGFβR2 gene.

In certain embodiments, the genetically-modified immune cell can further comprise in its genome a nucleic acid sequence encoding a engineered antigen receptor (e.g., a CAR or an exogenous TCR). In some embodiments, the genetically-modified immune cell can further comprise in its genome a nucleic acid sequence encoding an HLA-E fusion protein capable of being expressed on the immune cell surface. Exemplary and non-limiting HLA-E fusion proteins useful in the present disclosure are described in PCT/US2020/026571.

The engineered antigen receptor nucleic acid sequence (e.g., a CAR/TCR-encoding nucleic acid sequence) and/or the nucleic acid sequence encoding the HLA-E fusion protein can be located within the same gene as the inhibitory agent-encoding sequence. Alternatively, the engineered antigen receptor nucleic acid sequence and/or nucleic acid sequence encoding the HLA-E fusion protein can be located within a different gene as the inhibitory agent-encoding sequence.

Each of the coding sequences (e.g., a coding sequence encoding an engineered antigen receptor and inhibitory agent) can be operably linked to different promoters. In other embodiments, the inhibitory agent-encoding sequence is operably linked to the same promoter as the nucleic acid sequence encoding the engineered antigen receptor (e.g., a CAR or exogenous TCR) and/or the nucleic acid sequence encoding the HLA-E fusion protein. In some specific examples, where a nucleic acid sequence encoding a inhibitory agent, a nucleic acid sequence encoding an engineered antigen receptor (e.g., a CAR or exogenous TCR) and a nucleic acid sequence encoding an HLA-E fusion protein are all located within the same gene, two of the nucleic acid sequences are operably linked to a first promoter, and the third nucleic acid sequence is operably linked to a second promoter. In other specific examples, where a nucleic acid sequence encoding a inhibitory agent, a nucleic acid sequence encoding an engineered antigen receptor (e.g., a CAR or exogenous TCR) and a nucleic acid sequence encoding an HLA-E fusion protein are all located within the same gene, all three of the nucleic acid sequences are operably linked to the same promoter. In various embodiments, the nucleic acid sequences can be operably linked to an endogenous promoter following insertion into the genome. In some such cases, the polynucleotides or template nucleic acids of the invention may not require an exogenous promoter in order for the encoded sequences to be expressed. Further, in such cases, the polynucleotides or template nucleic acids may comprise elements (e.g., splice acceptor sequences, 2A or IRES sequences, and the like) necessary for the nucleic acids to be operably linked to the endogenous promoter. In other embodiments, the polynucleotides or template nucleic acids of the invention comprise one or more exogenous promoters that are operably linked to the nucleic acid sequences and drive expression of the inhibitory agent, the engineered antigen receptor (e.g., a CAR or exogenous TCR), and/or HLA-E fusion protein.

Each of the coding sequences can be present in the genome in the same orientation or in different orientations from each other. For example, one coding sequence can be on the plus strand of the double-stranded DNA and another coding sequence on the minus strand. In some embodiments, the inhibitory agent-encoding nucleic acid sequence is 3′ downstream of the nucleic acid sequence encoding the engineered antigen receptor (e.g., a CAR or exogenous TCR) and/or the nucleic acid sequence encoding the HLA-E fusion protein. In alternative embodiments, the inhibitory agent-encoding sequence is 5′ upstream of the engineered antigen receptor-encoding sequence (e.g., CAR/TCR) and/or the nucleic acid sequence encoding the HLA-E fusion protein.

In certain embodiments, nucleic acid sequences, such as those encoding an engineered antigen receptor (e.g., a CAR or exogenous TCR), a inhibitory agent, and/or an HLA-E fusion protein, are operably linked to the same promoter and are separated by any element known in the art to allow for the translation of two or more genes (i.e., cistrons) from the same polynucleotide. Such elements can include, but are not limited to, an IRES element, a T2A element, a P2A element (e.g., P2A/furin), an E2A element, and an F2A element.

In certain embodiments, the genetically-modified immune cell comprises a nucleic acid sequence encoding a cell-surface protein that protects the immune cell from NK cell killing. In some examples, the nucleic acid sequence encodes a non-classical MHC I protein. Non-classical MHC class I proteins can include, without limitation, HLA-E, HLA-F, HLA-G, and HLA-H. In particular examples, the nucleic acid sequence encodes an HLA-E protein. Examples of HLA-E proteins include, without limitation, an HLA-E-01:01 protein or an HLA-E-01:03 protein. In particular examples of the invention, the nucleic acid sequence encodes a fusion protein comprising a non-classical MHC class I protein (e.g., HLA-E) and at least one additional protein that enables expression of the MHC class I protein on the cell-surface of the immune cell. The fusion protein can comprise, for example, an MHC class I protein (e.g., HLA-E) fused to a beta-2 microglobulin protein that enables expression of the MHC class I protein on the cell-surface. In further examples, the fusion protein can comprise a non-classical MHC class I protein (e.g., HLA-E) fused to both a beta-2 microglobulin protein and an additional protein that is presented extracellularly by the non-classical MHC. Such additional proteins can include, for example, an HLA-G leader peptide. The individual proteins of the fusion protein can be fused by polypeptide linkers such as, for example, a linker described in PCT/US2020/026571.

In specific embodiments, the fusion protein is an HLA-E fusion protein comprising an HLA-E protein, a beta-2 microglobulin protein, and an HLA-G leader peptide. In some such embodiments, the HLA-E protein is an HLA-E-01:01 protein or an HLA-E-01:03 protein.

In certain embodiments, the genetically-modified eukaryotic cell comprises a polynucleotide (e.g., an expression cassette) comprising a nucleic acid sequence encoding an engineered antigen receptor (e.g., a CAR or an engineered TCR) and a shRNAmiR integrated in its genome. Exemplary and non-limiting polynucleotides and orientations that are useful for modifying eukaryotic cells (e.g., immune cells) are described in PCT/US2020/026571, which are incorporated by reference herein.

Generally, an engineered antigen receptor (e.g., a CAR) of the present disclosure will comprise at least an extracellular domain, a transmembrane domain, and an intracellular domain. In some embodiments, the extracellular domain comprises a target-specific binding element otherwise referred to as an extracellular ligand-binding domain or moiety. In some embodiments, the intracellular domain, or cytoplasmic domain, comprises at least one co-stimulatory domain and one or more signaling domains.

In some embodiments, an engineered antigen receptor (e.g., a CAR or engineered TCR) useful in the invention comprises an extracellular ligand-binding domain. The choice of ligand-binding domain depends upon the type and number of ligands that define the surface of a target cell. For example, the ligand-binding domain may be chosen to recognize a ligand that acts as a cell surface marker on target cells associated with a particular disease state. Thus, some examples of cell surface markers that may act as ligands for the ligand-binding domain in an engineered antigen receptor (e.g., a CAR or engineered TCR) can include those associated with viruses, bacterial and parasitic infections, autoimmune disease, and cancer cells. In some embodiments, an engineered antigen receptor (e.g., a CAR or engineered TCR) is engineered to target a cancer-specific antigen of interest by way of engineering a desired ligand-binding moiety that specifically binds to an antigen on a cancer (i.e., tumor) cell. In the context of the present disclosure, “cancer antigen,” tumor antigen,” “cancer-specific antigen,” or “tumor-specific antigen” refer to antigens that are common to specific hyperproliferative disorders such as cancer.

In some embodiments, the extracellular ligand-binding domain of the engineered antigen receptor (e.g., CAR or engineered TCR) is specific for any antigen or epitope of interest, particularly any tumor antigen or epitope of interest. As non-limiting examples, in some embodiments the antigen of the target is a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD22, CD30, CD40, CD79B, IL1RAP, glypican 3 (GPC3), CLL-1, disialoganglioside GD2, ductal-epithelial mucine, gp36, TAG-72, glycosphingolipids, glioma-associated antigen, B-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-1a, p53, prostein, PSMA, surviving and telomerase, prostate-carcinoma tumor antigen-1 (PCTA-1), MAGE, ELF2M, neutrophil elastase, ephrin B2, insulin growth factor (IGF1)-1, IGF-II, IGFI receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the A1 domain of tenascin-C (TnC A1) and fibroblast associated protein (fap); a lineage-specific or tissue specific antigen such as CD3, CD4, CD8, CD24, CD25, CD33, CD34, CD38, CD123, CD133, CD138, CTLA-4, B7-1 (CD80), B7-2 (CD86), endoglin, a major histocompatibility complex (MHC) molecule, BCMA (CD269, TNFRSF 17), CS1, or a virus-specific surface antigen such as an HIV-specific antigen (such as HIV gpl20); an EBV-specific antigen, a CMV-specific antigen, a HPV-specific antigen such as the E6 or E7 oncoproteins, a Lasse Virus-specific antigen, an Influenza Virus-specific antigen, as well as any derivate or variant of these surface markers.

In some examples, the extracellular ligand-binding domain or moiety is an antibody, or antibody fragment. An antibody fragment can, for example, be at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CHi domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some such embodiments, the scFv can comprise a heavy chain variable (VH) domain and a light chain variable (VL) domain from a monoclonal antibody having specificity for an antigen. In some embodiments, the scFv is attached via a linker sequence. In some embodiments, the scFv is murine, humanized, or fully human.

The extracellular ligand-binding domain of a chimeric antigen receptor can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention.

In some embodiments, the extracellular domain of a chimeric antigen receptor can comprise a naturally-occurring ligand for an antigen of interest, or a fragment of a naturally-occurring ligand which retains the ability to bind the antigen of interest.

An engineered antigen receptor (e.g., a CAR) can comprise a transmembrane domain which links the extracellular ligand-binding domain with the intracellular signaling and co-stimulatory domains via a hinge region or spacer sequence. The transmembrane domain can be derived from any membrane-bound or transmembrane protein. For example, the transmembrane polypeptide can be a subunit of the T-cell receptor (e.g., an α, β, γ or ζ polypeptide constituting CD3 complex), IL2 receptor p55 (α chain), p75 (β chain) or γ chain, subunit chain of Fc receptors (e.g., Fcy receptor III) or CD proteins such as the CD8 alpha chain. In certain examples, the transmembrane domain is a CD8 alpha domain. Alternatively, the transmembrane domain can be synthetic and can comprise predominantly hydrophobic residues such as leucine and valine.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcγRllla receptor or IgG1. In certain examples, the hinge region can be a CD8 alpha domain.

Intracellular signaling domains of an engineered antigen receptor (e.g., a CAR) are responsible for activation of at least one of the normal effector functions of the cell in which the engineered antigen receptor has been placed and/or activation of proliferative and cell survival pathways. The term “effector function” refers to a specialized function of a cell. Effector function of a T cell, for example, may be cytolytic activity or helper activity including the secretion of cytokines. The intracellular stimulatory domain can include one or more cytoplasmic signaling domains that transmit an activation signal to the T cell following antigen binding. Such cytoplasmic signaling domains can include, without limitation, a CD3 zeta signaling domain.

The intracellular stimulatory domain can also include one or more intracellular co-stimulatory domains that transmit a proliferative and/or cell-survival signal after ligand binding. In some cases, the co-stimulatory domain can comprise one or more TRAF-binding domains. Such intracellular co-stimulatory domains can be any of those known in the art and can include, without limitation, those co-stimulatory domains disclosed in WO 2018/067697 including, for example, Novel 6 (“N6”). Further examples of co-stimulatory domains can include 4-1BB (CD137), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof. In a particular embodiment, the co-stimulatory domain is an N6 domain. In another particular embodiment, the co-stimulatory domain is a 4-1BB co-stimulatory domain.

In other embodiments, the genetically-modified immune cell comprises a nucleic acid sequence encoding an exogenous T cell receptor (TCR). Such exogenous T cell receptors can comprise alpha and beta chains or, alternatively, may comprise gamma and delta chains. Exogenous TCRs useful in the invention may have specificity to any antigen or epitope of interest such as, without limitation, any antigen or epitope disclosed herein.

In particular embodiments, the engineered antigen receptor (e.g., a CAR or a exogenous TCR) can be specific for any type of cancer cell. Such cancers can include, without limitation, carcinoma, lymphoma, sarcoma, blastomas, leukemia, cancers of B cell origin, breast cancer, gastric cancer, neuroblastoma, osteosarcoma, lung cancer, melanoma, prostate cancer, colon cancer, renal cell carcinoma, ovarian cancer, rhabdomyosarcoma, leukemia, and Hodgkin lymphoma. In specific embodiments, cancers and disorders include but are not limited to pre-B ALL (pediatric indication), adult ALL, mantle cell lymphoma, diffuse large B cell lymphoma, salvage post allogenic bone marrow transplantation, and the like. These cancers can be treated using a combination of CARs that target, for example, CD19, CD20, CD22, and/or ROR1. In some non-limiting examples, a genetically-modified immune cell or population thereof of the present disclosure targets carcinomas, lymphomas, sarcomas, melanomas, blastomas, leukemias, and germ cell tumors, including but not limited to cancers of B-cell origin, neuroblastoma, osteosarcoma, prostate cancer, renal cell carcinoma, liver cancer, gastric cancer, bone cancer, pancreatic cancer, skin cancer, cancer of the head or neck, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, multiple myeloma, Hodgkin lymphoma, non-Hodgkin lymphomas, acute myeloid lymphoma, chronic myelogenous leukemia, chronic lymphoid leukemia, immunoblastic large cell lymphoma, acute lymphoblastic leukemia, mycosis fungoides, anaplastic large cell lymphoma, and T-cell lymphoma, and any combinations of said cancers. In certain embodiments, cancers of B-cell origin include, without limitation, B-lineage acute lymphoblastic leukemia, B-cell chronic lymphocytic leukemia, B-cell lymphoma, diffuse large B cell lymphoma, pre-B ALL (pediatric indication), mantle cell lymphoma, follicular lymphoma, marginal zone lymphoma, Burkitt's lymphoma, multiple myeloma, and B-cell non-Hodgkin lymphoma. In some examples, cancers can include, without limitation, cancers of B cell origin or multiple myeloma. In some examples, the cancer of B cell origin is acute lymphoblastic leukemia (ALL), chronic lymphocytic leukemia (CLL), small lymphocytic lymphoma (SLL), or non-Hodgkin lymphoma (NHL). In some examples, the cancer of B cell origin is mantle cell lymphoma (MCL) or diffuse large B cell lymphoma (DLBCL).

In some embodiments, genetically-modified immune cells of the invention comprise an inactivated TCR alpha gene and/or an inactivated TCR beta gene. Inactivation of the TCR alpha gene and/or TCR beta gene to generate the genetically-modified cells of the present invention occurs in at least one or both alleles where the TCR alpha gene and/or TCR beta gene is being expressed. Accordingly, inactivation of one or both genes prevents expression of the endogenous TCR alpha chain or the endogenous TCR beta chain protein. Expression of these proteins is required for assembly of the endogenous alpha/beta TCR on the cell surface. Thus, inactivation of the TCR alpha gene and/or the TCR beta gene results in genetically-modified immune that have no detectable cell surface expression of the endogenous alpha/beta TCR. The endogenous alpha/beta TCR incorporates CD3. Therefore, cells with an inactivated TCR alpha gene and/or TCR beta chain can have no detectable cell surface expression of CD3. In particular embodiments, the inactivated gene is a TCR alpha constant region (TRAC) gene.

In some examples, the TCR alpha gene, the TRAC gene, or the TCR beta gene is inactivated by insertion of a template nucleic acid into a cleavage site in the gene. Insertion of the template nucleic acid disrupts expression of the endogenous TCR alpha chain or TCR beta chain and, therefore, prevents assembly of an endogenous alpha/beta TCR on the T cell surface. In some examples, the template nucleic acid is inserted into the TRAC gene. In a particular example, a template nucleic acid is inserted into the TRAC gene at an engineered meganuclease recognition sequence comprising SEQ ID NO: 20 (i.e., the TRC 1-2 recognition sequence). In particular examples, the CAR transgene is inserted into SEQ ID NO: 20 between nucleotide positions 13 and 14.

In some of those embodiments wherein the genetically-modified immune cell expresses a CAR or exogenous TCR, such cells have no detectable cell-surface expression of an endogenous T cell receptor (e.g., an alpha/beta T cell receptor). Thus, the invention further provides a population of genetically-modified immune cells that express a inhibitory agent and have no detectable cell-surface expression of an endogenous T cell receptor (e.g., an alpha/beta T cell receptor), and in some embodiments also express a CAR or exogenous TCR. For example, the population can include a plurality of genetically-modified immune cells of the invention which express a CAR (i.e., are CAR+), or an exogenous T cell receptor (i.e., exoTCR+), and have no cell-surface expression of an endogenous T cell receptor (i.e., are TCR−).

As used herein, “detectable cell-surface expression of an endogenous TCR” refers to the ability to detect one or more components of the TCR complex (e.g., an alpha/beta TCR complex) on the cell surface of an immune cell using standard experimental methods. Such methods can include, for example, immunostaining and/or flow cytometry specific for components of the TCR itself, such as a TCR alpha or TCR beta chain, or for components of the assembled cell-surface TCR complex, such as CD3. Methods for detecting cell-surface expression of an endogenous TCR (e.g., an alpha/beta TCR) on an immune cell include those described in the examples herein, and, for example, those described in MacLeod et al. (2017) Molecular Therapy 25(4): 949-961.

### 2.4 Target Proteins

In some embodiments, the genetically-modified eukaryotic cells (e.g., genetically-modified immune cells) of the presently disclosed compositions and methods can comprise a nucleic acid sequence encoding an inhibitory agent that reduces expression of a target protein. In some embodiments, the inhibitory agent reduces expression of TGFβ1. In some other embodiments, the inhibitory agent reduces expression of TGFβR2. In some further embodiments, the inhibitory agent reduces expression of TGFβR1.

In some embodiments, the genetically-modified eukaryotic cells (e.g., genetically-modified immune cells) of the presently disclosed compositions and methods have a target protein knocked out through an engineered nuclease described herein. In some embodiments, the genetically modified cell has TGFβ1 knocked out. In some other embodiments, the genetically modified cell has TGFβR2 knocked out. In some further embodiments, the genetically modified cell has TGFβR1 knocked out.

In some embodiments, the endogenous protein with reduced expression levels as the result of the expression of an inhibitory agent is transforming growth factor beta 1 (TGFβ1).

In some embodiments, the expression of TGFβ1 is reduced by at least about 10%, about 20%, about 30%, about 40%, about 50%, about 55%, about 60%, about 65%, about 70%, about 75%, about 80%, about 85%, about 90%, about 95%, or up to about 99% compared to a control cell (e.g., a cell not expressing a TGFβ1-targeted inhibitory agent such as a shRNAmiR). In some embodiments the expression of a TGFβ1 protein is reduced by about 50% to about 99%, by about 55% to about 99%, by about 60% to about 99%, by about 65% to about 99%, by about 70% to about 99%, by about 75% to about 99%, by about 80% to about 99%, by about 85% to about 99%, by about 90% to about 99%, by about 95% to about 99%, by about 75% to about 95%, by about 80% to about 95%, by about 85% to about 95%, or by about 90% to about 95%, compared to a control cell.

In some embodiments, the inhibitory agent is a shRNAmiR specific to TGFβ1. The shRNAmiR molecule may target any region of a TGFβ1 mRNA. Representative TGFβ1 mRNA and protein sequences are known in the art. A non-limiting example of a TGFβ1 mRNA sequence is NCBI Acc. No. NM_000660.7 and a TGFβ1 protein sequence is NCBI Acc. No. NP_000651.3.

shRNAmiR molecules that target TGFβ1 may comprise any passenger and corresponding guide sequence that is complementary (fully or partially) to a sequence within the TGFβ1 gene. In some embodiments, the guide and passenger sequence of the shRNAmiR comprise the sequences set forth as SEQ ID NO: 8 and 9, respectively (e.g., TGFβ1 72173 shRNAmiR). In other embodiments, the guide and passenger sequence of the shRNAmiR comprise the sequences set forth as SEQ ID NO: 10 and 11, respectively (e.g., TGFβ1 72174 shRNAmiR). In still other embodiments, the guide and passenger sequence of the shRNAmiR comprise the sequences set forth as SEQ ID NO: 12 and 13, respectively (e.g., TGFβ1 72175 shRNAmiR). In yet other embodiments, the guide and passenger sequence of the shRNAmiR comprise the sequences set forth as SEQ ID NO: 14 and 15, respectively (e.g., TGFβ1 72176 shRNAmiR).

The TGFβ1-targeted shRNAmiR may comprise a sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or more sequence identity to the nucleic acid sequence set forth in any one of SEQ ID NOs: 16-19. In particular embodiments, the shRNAmiR comprises the sequence set forth in any one of SEQ ID NOs: 16-19. In some embodiments, the shRNAmiR comprises the sequence set forth in SEQ ID NO: 16. In some embodiments, the shRNAmiR comprises the sequence set forth in SEQ ID NO: 17. In some embodiments, the shRNAmiR comprises the sequence set forth in SEQ ID NO: 18. In some embodiments, the shRNAmiR comprises the sequence set forth in SEQ ID NO: 19.

In some of the embodiments wherein the genetically-modified eukaryotic cell (e.g., a genetically-modified immune cell) expresses an inhibitory agent that reduces the expression of TGFβ1, the genetically-modified immune cell is less susceptible to immunosuppression by autocrine TGFβ1 signaling compared to a control cell (e.g., an immune cell not expressing a TGFβ1-targeted inhibitory agent such as a shRNAmiR). As used herein, the term “immunosuppression” refers to the reduction of the activation or efficacy of the immune system. Immunosuppression by TGFβ1 can be measured using any method known in the art, including but not limited to measuring the effects of TGFβ1 on T cell differentiation and/or cytokine production.

In some embodiments, the endogenous protein with reduced expression levels as the result of the expression of an inhibitory agent is transforming growth factor beta receptor 2 (TGFβR2). TGFβR2 is a transmembrane receptor that binds transforming growth factor-beta (TGFβ1). TGFβR2 comprises a serine/threonine protein kinase domain and heterodimerizes with other TGFβ receptors.

In some embodiments, the cell surface expression of TGFβR2 is reduced by at least about 10%, about 20%, about 30%, about 40%, about 50%, about 55%, about 60%, about 65%, about 70%, about 75%, about 80%, about 85%, about 90%, about 95%, or up to about 99% compared to a control cell (e.g., a cell not expressing a TGFβR2-targeted inhibitory agent). In some embodiments the expression of a TGFβR2 protein is reduced by about 50% to about 99%, by about 55% to about 99%, by about 60% to about 99%, by about 65% to about 99%, by about 70% to about 99%, by about 75% to about 99%, by about 80% to about 99%, by about 85% to about 99%, by about 90% to about 99%, by about 95% to about 99%, by about 75% to about 95%, by about 80% to about 95%, by about 85% to about 95%, or by about 90% to about 95%, compared to a control cell.

In some embodiments, the inhibitory agent is a shRNAmiR specific to TGFβR2. The shRNAmiR molecule may target any region of a TGFβR2 mRNA. Representative TGFβR2mRNA and protein sequences are known in the art. A non-limiting example of a TGFBR2 mRNA sequence is NM_001024847.2 and a TGFBR2 protein sequence is NCBI Ace. No. NP_001020018.1.

shRNAmiR molecules that target TGFβR2 may comprise any passenger and corresponding guide sequence that is complementary (fully or partially) to a sequence within the TGFβR2 gene, such sequences have previously been described in PCT/US2020/026571.

In some of the embodiments wherein the genetically-modified eukaryotic cell (e.g., a genetically modified immune cell) expresses a shRNAmiR that reduces the expression of TGFβR2, the genetically-modified eukaryotic cell is less susceptible to immunosuppression by transforming growth factor TGFβ1 (TGFβ1) compared to a control cell (e.g., an eukaryotic cell not expressing a TGFβR2-targeted inhibitory agent).

### 2.5 Methods for Reducing Expression of Endogenous Proteins

The present invention provides methods for reducing the expression of a TGFβ signaling protein in an eukaryotic cell by introducing into the cell a template nucleic acid comprising a nucleic acid sequence encoding an inhibitory agent (e.g., an inhibitory nucleic acid molecule such as a shRNAmiR), whereby the template nucleic acid is inserted into the genome and expressed.

The template nucleic acid can be inserted into the genome of the eukaryotic cell by random integration. Alternatively, the template nucleic acid can be inserted into a target gene by nuclease-mediated targeted insertion, wherein an engineered nuclease has specificity for a recognition sequence in the genome of the eukaryotic cell and generates a cleavage site at the recognition sequence, allowing for the insertion of the template nucleic acid into the genome of the eukaryotic cell at the cleavage site.

In addition, in some embodiments, an endogenous protein can be knocked out by methods known in the art (e.g, a TGFβ signaling molecule, such as TGFβ1, TGFβR1, and/or TGFβR2). It is known in the art that it is possible to use a site-specific nuclease to make a DNA break in the genome of a living cell, and that such a DNA break can result in permanent modification of the genome via mutagenic NHEJ repair or via homologous recombination with a transgenic DNA sequence. NHEJ can produce mutagenesis at the cleavage site, resulting in inactivation of the allele. NHEJ-associated mutagenesis may inactivate an allele via generation of early stop codons, frameshift mutations producing aberrant non-functional proteins, insertions, deletions, or could trigger mechanisms such as nonsense-mediated mRNA decay. The use of nucleases to induce mutagenesis via NHEJ can be used to target a specific mutation or a sequence present in a wild-type allele.

In addition, engineered nucleases disclosed herein can be used for targeted insertion of the template nucleic acid. Exemplary and non-limiting nucleases that can be used for knock out of a gene or insertion of a template nucleic acid within a gene include an engineered meganuclease, a zinc finger nuclease, a TALEN, a compact TALEN, a CRISPR system nuclease, or a megaTAL.

In particular embodiments, the recognition sequence of the engineered nuclease is within a target gene. The target gene can be any gene in which the sequence is desired to be altered (e.g., addition or subtraction of a nucleotide, substitution of a nucleotide, or insertion of a heterologous or exogenous sequence). For example, knockout of a target gene by genetic inactivation may be desired. In some embodiments, the target gene is a TCR alpha gene or a TCR beta gene. In particular embodiments, the target gene can be the TCR alpha constant region (TRAC) gene. In some specific embodiments, the target gene is the TRAC gene and the recognition sequence is the TRC 1-2 recognition sequence set forth in SEQ ID NO: 20.

In some of these embodiments, the insertion of the template nucleic acid into the target gene leads to disruption of expression of the full-length endogenous protein encoded by the target gene. Thus, in some of those embodiments wherein the target gene is a TCR alpha gene, TRAC gene, or TCR beta gene, the genetically-modified eukaryotic cell (e.g., a genetically-modified immune cell) does not have detectable cell-surface expression of an endogenous TCR, such as an alpha/beta TCR, because the endogenous TCR will not properly assemble at the cell surface in the absence of the endogenous proteins encoded by these genes.

In particular embodiments in which the genetically-modified eukaryotic cell does not have detectable cell-surface expression of an endogenous TCR (e.g., an alpha/beta TCR) due to inactivation of a gene encoding a component of an alpha/beta TCR, the genetically-modified eukaryotic cell further expresses an engineered antigen receptor (e.g., a CAR or exogenous TCR) and/or an HLA-E fusion protein. The engineered antigen receptor can be encoded by sequences comprised within the template nucleic acid. In some of these embodiments, the engineered antigen receptor protein-encoding sequence is operably linked to a different promoter than the inhibitory agent-encoding sequence. In alternative embodiments, the engineered antigen receptor-encoding sequence is operably linked to the same promoter, or to a different promoter, as the inhibitory agent-encoding sequence. The engineered antigen receptor-encoding sequence can be 5′ or 3′ of the inhibitory agent-encoding sequence, and the coding sequences can be in the same or different orientation, such as 5′ to 3′ or 3′ to 5′. Further, the coding sequences may be separated by an element known in the art to allow for the translation of two or more genes (i.e., cistrons) from the same polynucleotide including, but not limited to, an IRES element, a T2A element, a P2A element (e.g., a P2A/furin), an E2A element, and an F2A element.

The use of nucleases for disrupting expression of an endogenous TCR has been disclosed, including the use of zinc finger nucleases (ZFNs), transcription activator-like effector nucleases (TALENs), megaTALs, and CRISPR systems (e.g., Osborn et al. (2016), Molecular Therapy 24(3): 570-581; Eyquem et al. (2017), Nature 543: 113-117; U.S. Pat. No. 8,956,828; U.S. Publication No. US2014/0301990; U.S. Publication No. US2012/0321667). The specific use of engineered meganucleases for cleaving DNA targets in the human TRAC gene has also been previously disclosed. For example, International Publication No. WO 2014/191527, which disclosed variants of the I-OnuI meganuclease that were engineered to target a recognition sequence within exon 1 of the TCR alpha constant region gene.

Moreover, in International Publication Nos. WO 2017/062439 and WO 2017/062451, Applicants disclosed engineered meganucleases which have specificity for recognition sequences in exon 1 of the TCR alpha constant region (TRAC) gene. These included “TRC 1-2 meganucleases” which have specificity for the TRC 1-2 recognition sequence (SEQ ID NO: 20) in exon 1 of the TRAC gene. The '439 and '451 publications also disclosed methods for targeted insertion of a CAR coding sequence or an exogenous TCR coding sequence into the TCR 1-2 meganuclease cleavage site.

In particular embodiments, the nucleases used to practice the invention are single-chain meganucleases. A single-chain meganuclease comprises an N-terminal subunit and a C-terminal subunit joined by a linker peptide. Each of the two domains recognizes half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs.

Engineered nucleases for knock out of a gene and/or insertion of a template nucleic acid can be delivered into a cell in the form of protein or, preferably, as a nucleic acid encoding the engineered nuclease. Such nucleic acids can be DNA (e.g., circular or linearized plasmid DNA or PCR products) or RNA (e.g., mRNA). For embodiments in which the engineered nuclease coding sequence is delivered in DNA form, it should be operably linked to a promoter to facilitate transcription of the nuclease gene. Mammalian promoters suitable for the invention include constitutive promoters such as the cytomegalovirus early (CMV) promoter (Thomsen et al. (1984), Proc Natl Acad Sci USA. 81(3):659-63) or the SV40 early promoter (Benoist and Chambon (1981), Nature. 290(5804):304-10) as well as inducible promoters such as the tetracycline-inducible promoter (Dingermann et al. (1992), Mol Cell Biol. 12(9):4038-45). A nucleic acid encoding an engineered nuclease can also be operably linked to a synthetic promoter. Synthetic promoters can include, without limitation, the JeT promoter (WO 2002/012514).

In certain embodiments, a nucleic acid sequence encoding an engineered nuclease is delivered on a recombinant DNA construct or polynucleotide. For example, the recombinant DNA construct can comprise an polynucleotide comprising a promoter and a nucleic acid sequence encoding an engineered nuclease described herein.

In some embodiments, mRNA encoding the engineered nuclease is delivered to the cell because this reduces the likelihood that the gene encoding the engineered nuclease will integrate into the genome of the cell.

The mRNA encoding an engineered nuclease can be produced using methods known in the art such as in vitro transcription. In some embodiments, the mRNA comprises a modified 5′ cap. Such modified 5′ caps are known in the art and can include, without limitation, an anti-reverse cap analogs (ARCA) (U.S. Pat. No. 7,074,596), 7-methyl-guanosine, CleanCap® analogs, such as Cap 1 analogs (Trilink; San Diego, CA), or enzymatically capped using, for example, a vaccinia capping enzyme or the like. In some embodiments, the mRNA may be polyadenylated. The mRNA may contain various 5′ and 3′ untranslated sequence elements to enhance expression of the encoded engineered nuclease and/or stability of the mRNA itself. Such elements can include, for example, posttranslational regulatory elements such as a woodchuck hepatitis virus posttranslational regulatory element.

The mRNA may contain nucleoside analogs or naturally-occurring nucleosides, such as pseudouridine, 5-methylcytidine, N6-methyladenosine, 5-methyluridine, or 2-thiouridine. Additional nucleoside analogs include, for example, those described in U.S. Pat. No. 8,278,036.

In another particular embodiment, a nucleic acid encoding an engineered nuclease can be introduced into the cell using a single-stranded DNA template. The single-stranded DNA can further comprise a 5′ and/or a 3′ AAV inverted terminal repeat (ITR) upstream and/or downstream of the sequence encoding the engineered nuclease. In other embodiments, the single-stranded DNA can further comprise a 5′ and/or a 3′ homology arm upstream and/or downstream of the sequence encoding the engineered nuclease.

In another particular embodiment, genes encoding a nuclease can be introduced into a cell using a linearized DNA template. In some examples, a plasmid DNA encoding a nuclease can be digested by one or more restriction enzymes such that the circular plasmid DNA is linearized prior to being introduced into a cell.

Purified nuclease proteins can be delivered into cells to cleave genomic DNA, which allows for homologous recombination or non-homologous end-joining at the cleavage site with a sequence of interest, by a variety of different mechanisms known in the art, including those further detailed herein below.

In some embodiments, nuclease proteins, or DNA/mRNA encoding the nuclease, are coupled to a cell penetrating peptide or targeting ligand to facilitate cellular uptake. Examples of cell penetrating peptides known in the art include poly-arginine (Jearawiriyapaisarn, et al. (2008) Mol Ther. 16:1624-9), TAT peptide from the HIV virus (Hudecz et al. (2005), Med. Res. Rev. 25: 679-736), MPG (Simeoni, et al. (2003) Nucleic Acids Res. 31:2717-2724), Pep-1 (Deshayes et al. (2004) Biochemistry 43: 7698-7706), and HSV-1 VP-22 (Deshayes et al. (2005) Cell Mol Life Sci. 62:1839-49). In an alternative embodiment, nuclease proteins, or DNA/mRNA encoding nucleases, are coupled covalently or non-covalently to an antibody that recognizes a specific cell-surface receptor expressed on target cells such that the nuclease protein/DNA/mRNA binds to and is internalized by the target cells. Alternatively, nuclease protein/DNA/mRNA can be coupled covalently or non-covalently to the natural ligand (or a portion of the natural ligand) for such a cell-surface receptor. (McCall, et al. (2014) Tissue Barriers. 2(4):e944449; Dinda, et al. (2013) Curr Pharm Biotechnol. 14:1264-74; Kang, et al. (2014) Curr Pharm Biotechnol. 15(3):220-30; Qian et al. (2014) Expert Opin Drug Metab Toxicol. 10(11):1491-508).

In some embodiments, nuclease proteins, or DNA/mRNA encoding nucleases, are coupled covalently or, preferably, non-covalently to a nanoparticle or encapsulated within such a nanoparticle using methods known in the art (Sharma, et al. (2014) Biomed Res Int. 2014). A nanoparticle is a nanoscale delivery system whose length scale is <1 μm, preferably <100 nm. Such nanoparticles may be designed using a core composed of metal, lipid, polymer, or biological macromolecule, and multiple copies of the nuclease proteins, mRNA, or DNA can be attached to or encapsulated with the nanoparticle core. This increases the copy number of the protein/mRNA/DNA that is delivered to each cell and, so, increases the intracellular expression of each nuclease to maximize the likelihood that the target recognition sequences will be cut. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012) Biomaterials. 33(30): 7621-30). Nanoparticles may additionally be advantageously coupled to targeting molecules to direct the nanoparticle to the appropriate cell type and/or increase the likelihood of cellular uptake. Examples of such targeting molecules include antibodies specific for cell-surface receptors and the natural ligands (or portions of the natural ligands) for cell surface receptors.

In some embodiments, the nuclease proteins or DNA/mRNA encoding the nucleases are encapsulated within liposomes or complexed using cationic lipids (see, e.g., Lipofectamine™, Life Technologies Corp., Carlsbad, CA; Zuris et al. (2015) Nat Biotechnol. 33: 73-80; Mishra et al. (2011) J Drug Deliv. 2011:863734). The liposome and lipoplex formulations can protect the payload from degradation, and facilitate cellular uptake and delivery efficiency through fusion with and/or disruption of the cellular membranes of the target cells.

In some embodiments, nuclease proteins, or DNA/mRNA encoding nucleases, are encapsulated within polymeric scaffolds (e.g., PLGA) or complexed using cationic polymers (e.g., PEI, PLL) (Tamboli et al. (2011) Ther Deliv. 2(4): 523-536). Polymeric carriers can be designed to provide tunable drug release rates through control of polymer erosion and drug diffusion, and high drug encapsulation efficiencies can offer protection of the therapeutic payload until intracellular delivery to the desired target cell population.

In some embodiments, nuclease proteins, or DNA/mRNA encoding recombinant nucleases, are combined with amphiphilic molecules that self-assemble into micelles (Tong et al. (2007) J Gene Med. 9(11): 956-66). Polymeric micelles may include a micellar shell formed with a hydrophilic polymer (e.g., polyethyleneglycol) that can prevent aggregation, mask charge interactions, and reduce nonspecific interactions.

In some embodiments, nuclease proteins, or DNA/mRNA encoding meganucleases, are formulated into an emulsion or a nanoemulsion (i.e., having an average particle diameter of <1 nm) for administration and/or delivery to the target cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in US Patent Application Nos. 2002/0045667 and 2004/0043041, and U.S. Pat. Nos. 6,015,832, 6,506,803, 6,635,676, and 6,559,189, each of which is incorporated herein by reference in its entirety.

In some embodiments, nuclease proteins, or DNA/mRNA encoding nucleases, are covalently attached to, or non-covalently associated with, multifunctional polymer conjugates, DNA dendrimers, and polymeric dendrimers (Mastorakos et al. (2015) Nanoscale. 7(9): 3845-56; Cheng et al. (2008) J Pharm Sci. 97(1): 123-43). The dendrimer generation can control the payload capacity and size, and can provide a high drug payload capacity. Moreover, display of multiple surface groups can be leveraged to improve stability, reduce nonspecific interactions, and enhance cell-specific targeting and drug release.

In some embodiments, polynucleotides comprising a nucleic acid sequence encoding an engineered meganuclease described herein are introduced into a cell using a recombinant virus (i.e., a recombinant viral vector). Such recombinant viruses are known in the art and include recombinant retroviruses, recombinant lentiviruses, recombinant adenoviruses, and recombinant adeno-associated viruses (AAVs) (reviewed in Vannucci, et al. (2013 New Microbiol. 36:1-22). Recombinant AAVs useful in the invention can have any serotype that allows for transduction of the virus into a target cell type and expression of the nuclease gene. In particular embodiments, recombinant AAVs have a serotype of AAV2 or AAV6. AAV vectors can also be self-complementary such that they do not require second-strand DNA synthesis in the host cell (McCarty, et al. (2001) Gene Ther. 8:1248-54). Polynucleotides delivered by recombinant AAV vectors, including those that deliver a template nucleic acid disclosed herein, can include left (5′) and right (3′) inverted terminal repeats.

If the nuclease genes are delivered in DNA form (e.g. plasmid) and/or via a viral vector (e.g. AAV) they must be operably linked to a promoter. In some embodiments, this can be a viral promoter such as endogenous promoters from the viral vector (e.g. the LTR of a lentiviral vector) or the well-known cytomegalovirus- or SV40 virus-early promoters. In a preferred embodiment, nuclease genes are operably linked to a promoter that drives gene expression preferentially in the target cell (e.g., a T cell).

The engineered antigen receptor coding sequence (e.g., CAR/TCR coding sequence) and/or the HLA-E fusion protein coding sequence can further comprise additional control sequences. For example, the sequence can include homologous recombination enhancer sequences, Kozak sequences, polyadenylation sequences, transcriptional termination sequences, selectable marker sequences (e.g., antibiotic resistance genes), origins of replication, and the like. Sequences encoding engineered nucleases can also include at least one nuclear localization signal. Examples of nuclear localization signals are known in the art (see, e.g., Lange et al., J. Biol. Chem., 2007, 282:5101-5105).

The invention further provides for the introduction of a template nucleic acid into a target gene. In some embodiments, the template nucleic acid comprises a 5′ homology arm and a 3′ homology arm flanking the elements of the insert. Such homology arms have sequence homology to corresponding sequences 5′ upstream and 3′ downstream of the nuclease recognition sequence where a cleavage site is produced. In general, homology arms can have a length of at least 50 base pairs, preferably at least 100 base pairs, and up to 2000 base pairs or more, and can have at least 90%, preferably at least 95%, or more, sequence homology to their corresponding sequences in the genome.

A template nucleic acid encoding an inhibitory agent disclosed herein (e.g., encoding a shRNAmiR), a nucleic acid encoding an engineered antigen receptor (e.g., a CAR or exogenous TCR), and/or an HLA-E fusion protein can be introduced into the cell by any of the means previously discussed. In a particular embodiment, the template nucleic acid is introduced by way of a recombinant virus (i.e., a recombinant viral vector), such as a recombinant lentivirus, a recombinant retrovirus, a recombinant adenovirus, or preferably a recombinant AAV (i.e., a recombinant AAV vector). Recombinant AAVs useful for introducing an exogenous nucleic acid (e.g., a template nucleic acid) can have any serotype that allows for transduction of the virus into the cell and insertion of the exogenous nucleic acid sequence into the cell genome. In particular embodiments, the recombinant AAV have a serotype of AAV2 or AAV6. The recombinant AAV can also be self-complementary such that they do not require second-strand DNA synthesis in the host cell.

In another particular embodiment, the template nucleic acid encoding an inhibitory agent disclosed herein (e.g., encoding a shRNAmiR), a nucleic acid encoding an engineered antigen receptor (e.g., a CAR or exogenous TCR), and/or an HLA-E fusion protein can be introduced into the cell using a single-stranded DNA template. The single-stranded DNA can comprise the exogenous sequence of interest and, in preferred embodiments, can comprise 5′ and 3′ homology arms to promote insertion of the nucleic acid sequence into the meganuclease cleavage site by homologous recombination. The single-stranded DNA can further comprise a 5′ AAV inverted terminal repeat (ITR) sequence 5′ upstream of the 5′ homology arm, and a 3′ AAV ITR sequence 3′ downstream of the 3′ homology arm.

In another particular embodiment, the template nucleic acid encoding an inhibitory agent disclosed herein (e.g., encoding a shRNAmiR) a nucleic acid encoding an engineered antigen receptor (e.g., a CAR or exogenous TCR), and/or an HLA-E fusion protein can be introduced into the cell by transfection with a linearized DNA template. In some examples, a plasmid DNA can be digested by one or more restriction enzymes such that the circular plasmid DNA is linearized prior to transfection into the cell.

Immune cells (e.g., T cells) modified by the present invention may require activation prior to introduction of a nuclease and/or an exogenous sequence of interest. For example, T cells can be contacted with anti-CD3 and anti-CD28 antibodies that are soluble or conjugated to a support (i.e., beads) for a period of time sufficient to activate the cells.

Genetically-modified eukaryotic cells of the invention can be further modified to express one or more inducible suicide genes, the induction of which provokes cell death and allows for selective destruction of the cells in vitro or in vivo. In some examples, a suicide gene can encode a cytotoxic polypeptide, a polypeptide that has the ability to convert a non-toxic pro-drug into a cytotoxic drug, and/or a polypeptide that activates a cytotoxic gene pathway within the cell. That is, a suicide gene is a nucleic acid that encodes a product that causes cell death by itself or in the presence of other compounds. A representative example of such a suicide gene is one that encodes thymidine kinase of herpes simplex virus. Additional examples are genes that encode thymidine kinase of varicella zoster virus and the bacterial gene cytosine deaminase that can convert 5-fluorocytosine to the highly toxic compound 5-fluorouracil. Suicide genes also include as non-limiting examples genes that encode caspase-9, caspase-8, or cytosine deaminase. In some examples, caspase-9 can be activated using a specific chemical inducer of dimerization (CID). A suicide gene can also encode a polypeptide that is expressed at the surface of the cell that makes the cells sensitive to therapeutic and/or cytotoxic monoclonal antibodies. In further examples, a suicide gene can encode recombinant antigenic polypeptide comprising an antigenic motif recognized by the anti-CD20 mAb Rituximab and an epitope that allows for selection of cells expressing the suicide gene. See, for example, the RQR8 polypeptide described in WO2013153391, which comprises two Rituximab-binding epitopes and a QBEnd10-binding epitope. For such a gene, Rituximab can be administered to a subject to induce cell depletion when needed. In further examples, a suicide gene may include a QBEnd10-binding epitope expressed in combination with a truncated EGFR polypeptide.

Variants of naturally-occurring nucleases and inhibitory nucleic sequences (e.g., microRNA sequences, including pre-miRNA and pri-miRNA sequences) can be used in the presently disclosed compositions and methods. As used herein, “variants” is intended to mean substantially similar sequences. A “variant” polypeptide is intended to mean a polypeptide derived from the “native” polypeptide by deletion or addition of one or more amino acids at one or more internal sites in the native protein and/or substitution of one or more amino acids at one or more sites in the native polypeptide. As used herein, a “native” polynucleotide or polypeptide comprises a parental sequence from which variants are derived. Variant polypeptides encompassed by the embodiments are biologically active. That is, they continue to possess the desired biological activity of the native protein. Such variants may result, for example, from human manipulation. Biologically active variants of a native polypeptide will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, sequence identity to the amino acid sequence of the native polypeptide, as determined by sequence alignment programs and parameters described elsewhere herein. A biologically active variant of a polypeptide may differ from that polypeptide or subunit by as few as about 1-40 amino acid residues, as few as about 1-20, as few as about 1-10, as few as about 5, as few as 4, 3, 2, or even 1 amino acid residue.

The polypeptides may be altered in various ways including amino acid substitutions, deletions, truncations, and insertions. Methods for such manipulations are generally known in the art. For example, amino acid sequence variants can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

For polynucleotides, a “variant” comprises a deletion and/or addition of one or more nucleotides at one or more sites within the native polynucleotide. One of skill in the art will recognize that variants of the nucleic acids of the embodiments will be constructed such that the open reading frame is maintained. For polynucleotides, conservative variants include those sequences that, because of the degeneracy of the genetic code, encode the amino acid sequence of one of the polypeptides of the embodiments. Variant polynucleotides include synthetically derived polynucleotides, such as those generated, for example, by using site-directed mutagenesis but which still encode a polypeptide or RNA. Generally, variants of a particular polynucleotide of the embodiments will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein. Variants of a particular polynucleotide (i.e., the reference polynucleotide) can also be evaluated by comparison of the percent sequence identity between the polypeptide encoded by a variant polynucleotide and the polypeptide encoded by the reference polynucleotide.

The deletions, insertions, and substitutions of the protein sequences encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by screening the polypeptide for its biological activity.

### 2.6 Pharmaceutical Compositions

In some embodiments, the invention provides a pharmaceutical composition comprising a genetically-modified eukaryotic cell of the invention, or a population of genetically-modified eukaryotic cells of the invention, and a pharmaceutically-acceptable carrier. Such pharmaceutical compositions can be prepared in accordance with known techniques. See, e.g., Remington, The Science and Practice of Pharmacy (21st ed. 2005). In the manufacture of a pharmaceutical formulation according to the invention, cells are typically admixed with a pharmaceutically acceptable carrier and the resulting composition is administered to a subject. The carrier must, of course, be acceptable in the sense of being compatible with any other ingredients in the formulation and must not be deleterious to the subject. In some embodiments, pharmaceutical compositions of the invention can further comprise one or more additional agents useful in the treatment of a disease in the subject. In additional embodiments, pharmaceutical compositions of the invention can further include biological molecules, such as cytokines (e.g., IL-2, IL-7, IL-15, and/or IL-21), which promote in vivo cell proliferation and engraftment of genetically-modified T cells. Pharmaceutical compositions comprising genetically-modified eukaryotic cells of the invention can be administered in the same composition as an additional agent or biological molecule or, alternatively, can be co-administered in separate compositions.

The present disclosure also provides genetically-modified eukaryotic cells, or populations thereof, described herein for use as a medicament. The present disclosure further provides the use of genetically-modified eukaryotic cells or populations thereof described herein in the manufacture of a medicament for treating a disease in a subject in need thereof. In one such aspect, the medicament is useful for cancer immunotherapy in subjects in need thereof.

Pharmaceutical compositions of the invention can be useful for treating any disease state that can be targeted by adoptive immunotherapy, and particularly T cell adoptive immunotherapy. In a particular embodiment, the pharmaceutical compositions and medicaments of the invention are useful in the treatment of cancer. Non-limiting examples of cancers which may be treated with the pharmaceutical compositions and medicaments of the present disclosure include, without limitation, various types of cancers described herein that can be targeted by an engineered antigen receptor (e.g., a CAR or exogenous TCR).

In some of these embodiments wherein cancer is treated with the presently disclosed genetically-modified eukaryotic cells or populations thereof, the subject administered the genetically-modified eukaryotic cells or populations thereof is further administered an additional therapeutic, such as radiation, surgery, or a chemotherapeutic agent.

The invention further provides a population of genetically-modified eukaryotic cells comprising a plurality of genetically-modified eukaryotic cells described herein, which comprise in their genome a nucleic acid sequence encoding an inhibitory agent (e.g., a shRNAmiR), wherein the exogenous nucleic acid sequence encoding the inhibitory agent can be inserted into a target gene, such as the TCR alpha gene or the TRAC gene, such that the cell has no detectable cell-surface expression of an endogenous TCR (e.g., an alpha/beta TCR). In some embodiments of the invention, a population of eukaryotic cells is provided wherein about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or up to 100%, of cells in the population are a genetically-modified eukaryotic cell described herein.

### 2.7 Methods of Administering Genetically-Modified Eukaryotic Cells

Another aspect disclosed herein is the administration of an effective amount of the genetically-modified eukaryotic (e.g., genetically-modified immune cells), or populations thereof, of the present disclosure to a subject in need thereof. In particular embodiments, the pharmaceutical compositions described herein are administered to a subject in need thereof.

For example, an effective amount of a population of cells can be administered to a subject having a disease. In particular embodiments, the disease can be cancer, and administration of the genetically-modified immune cells of the invention represent an immunotherapy. The administered cells are able to reduce the proliferation, reduce the number, or kill target cells in the recipient. Unlike antibody therapies, genetically-modified immune cells of the present disclosure are able to replicate and expand in vivo, resulting in long-term persistence that can lead to sustained control of a disease.

Examples of possible routes of administration include parenteral, (e.g., intravenous (IV), intramuscular (IM), intradermal, subcutaneous (SC), or infusion) administration.

Moreover, the administration may be by continuous infusion or by single or multiple boluses.

In specific embodiments, the agent is infused over a period of less than about 12 hours, 6 hours, 4 hours, 3 hours, 2 hours, or 1 hour. In still other embodiments, the infusion occurs slowly at first and then is increased over time.

In some embodiments, a genetically-modified immune cell or population thereof of the present disclosure targets a tumor antigen for the purposes of treating cancer. Such cancers can include, without limitation, various types of cancers described herein that can be targeted by an engineered antigen receptor (e.g., a CAR or exogenous TCR).

When an “effective amount” or “therapeutic amount” is indicated, the precise amount to be administered can be determined by a physician with consideration of individual differences in age, weight, tumor size (if present), extent of infection or metastasis, and condition of the patient (subject). In some embodiments, a pharmaceutical composition comprising the genetically-modified eukaryotic cells or populations thereof described herein is administered at a dosage of 10⁴ to 10⁹ cells/kg body weight, including all integer values within those ranges. In further embodiments, the dosage is 10⁵ to 10⁶ cells/kg body weight, including all integer values within those ranges. In some embodiments, cell compositions are administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In some embodiments, administration of genetically-modified eukaryotic cells or populations thereof of the present disclosure reduce at least one symptom of a target disease or condition. For example, administration of genetically-modified T cells or populations thereof of the present disclosure can reduce at least one symptom of a cancer. Symptoms of cancers are well known in the art and can be determined by known techniques.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

Use of a shRNAmiR to Reduce TGFβ1 Production by CAR T Cells

### 1. Methods

T cells were isolated from an apheresis product and prepared for editing with the TRC 1-2 L.1592 engineered meganuclease, which binds and cleaves the TRC 1-2 recognition sequence (SEQ ID NO: 20) in the TRAC gene as previously described in WO/2019/200122, which is incorporated by reference herein in its entirety. Briefly, samples containing 1×10⁶ stimulated T cells were electroporated with 1 μg of RNA (TriLink) encoding the TRC1-2 L.1592 meganuclease using the Lonza 4D NucleoFector. T cells were immediately transduced with AAV6 vectors at an MOI of 2×10⁴ viral genomes/cell. T cells received either AAV 72155, which encodes a JeT promoter-driven BCMA-specific CAR and a β2m-specific shRNAmiR, or one of four experimental AAV6 vectors (72173-72176), each of which encode a JeT promoter-driven BCMA specific CAR and one of four experimental TGFβ1-specific shRNAmiR sequences provided in Table 1 below.

TABLE 1
Exemplary shRNAmiR Sequences

| Vector | | SEQ | |
|---|---|---|---|
| No. | | ID NO: | Sequence |
| 72173 | Guide | 8 | TTACAGCGAGATGTCATTTCCC |
| | Passenger | 9 | AGGAAATGACATCTCGCTGTAA |
| 72174 | Guide | 10 | TCATAGATTTCGTTGTGGGTTT |
| | Passenger | 11 | CAACCCACAACGAAATCTATGA |
| 72175 | Guide | 12 | ATGCTGTGTGTACTCTGCTTGA |
| | Passenger | 13 | CCAAGCAGAGTACACACAGCAT |
| 72176 | Guide | 14 | GTAGTGAACCCGTTGATGTCCA |
| | Passenger | 15 | CGGACATCAACGGGTTCACTAC |

The T cells were expanded for 5 days followed by CD3 depletion of non-edited cells as described in WO/2019/200122. CAR⁺ cells were identified in all cultures using a recombinant BCMA protein labeled with FITC (Acro Biosystems) and sorted using a Becton-Dickinson FACSMelody. RNA was prepared using the RNeasy PLUS kit (QIAgen), reverse-transcribed, and TGFβ1 transcript levels were quantified by RT-PCR. RNA was reverse-transcribed using the iScript cDNA synthesis kit (BioRad). Amplification was carried out using PerfeCTa qPCR ToughMix (Quant Bio) and pre-validated assays for TGFβ1 (TaqMan: ThermoFisher) and GAPDH (Integrated DNA Technologies).

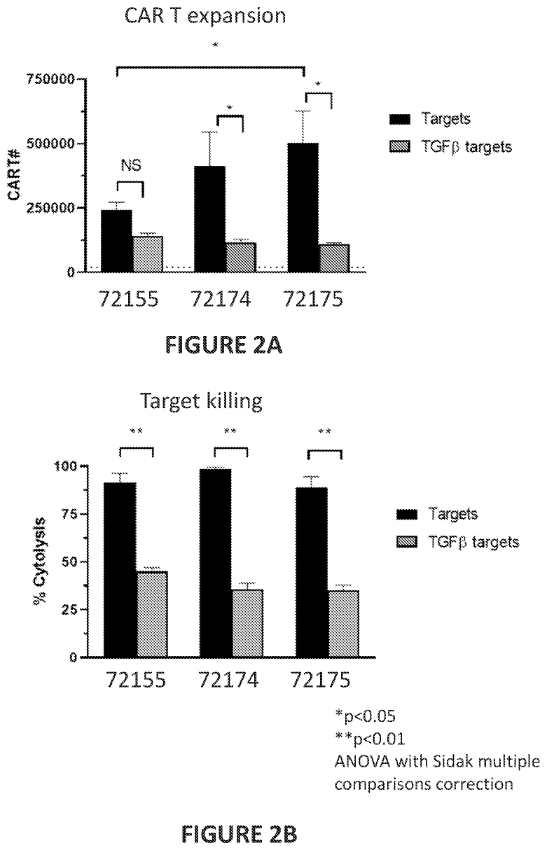

CD3-depleted CAR T cultures were challenged with tumor targets at a 1:1 ratio where 1=20000 cells (represented by horizontal dashed line in FIG. 2A). The tumor targets used in this study were K562 cells engineered to express BCMA (KBCMA; referred to as “Targets” in FIG. 2A and FIG. 2B) or KBCMA cells engineered to secrete active TGFβ1 (referred to as “TGFβ targets” in FIG. 2A and FIG. 2B). After 6 days of co-culture, samples were taken from each culture and stained with anti-CD4-APC (BioLegend), anti-CD8-BV421 (BioLegend), and anti-BCMA-PE (BioLegend) to enumerate T cells and surviving target cells in culture. Cytometric analysis was performed using a Beckman-Coulter CytoFLEX-LX cytometer.

### 2. Results

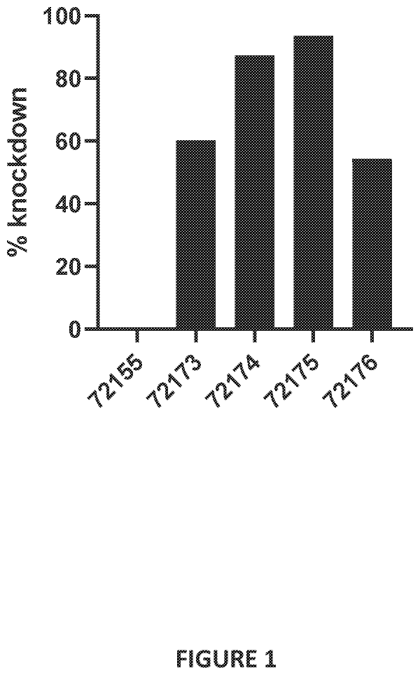

Quantitative RT-PCR was performed to determine the knockdown efficiency of each TGFβ1-specific shRNAmiR. As shown in FIG. 1, the percent (%) knockdown was calculated using GAPDH reference gene measurements, and the calibrator sample (72155 control CAR T cells) was set to 0% knockdown. The four candidate TGFβ1 shRNAmiR sequences exhibited reductions in TGFβ1 RNA levels ranging from 54%-93%. The two candidate sequences supporting the greatest knockdown (72174 at 87% and 72175 at 93%) were selected for cell function analyses.

All CAR T variants were found to expand vigorously in response to KBMCA encounter at d6 after plating with KBCMA or KBCMA-TGFβ target cells (E:T 1:1). Control 72155 CAR T cells expanded in excess of 10-fold while CAR T cells expressing a TGFβ1-specific shRNAmiR expanded 20-fold (72174) to 25-fold (72175) (FIG. 2A). ANOVA and hypothesis testing using Sidak's multiple comparisons correction indicates that the shRNAmiR demonstrating the most efficient TGFβ1 knockdown supports a significant proliferative advantage in response to target cell encounter (FIG. 2A). In contrast, all CAR T variants exhibited more modest expansion in response to TGFβ1-secreting KBMCAs with 72174 and 72175 exhibiting significantly reduced CAR T cell counts (FIG. 2A).

In line with the proliferation results, killing of KBCMA-TGFβ targets was significantly inhibited in all three CAR T variants (FIG. 2B). No differences in killing of control KBCMA targets were observed in TGFβ1-knockdown CAR T cells relative to control CAR T cells.

### 3. Conclusions

Reducing the abundance of TGFβ1 mRNA is associated with enhanced in vitro performance in BCMA-specific CAR T cells. Specifically, proliferation following antigen encounter is increased by 2-3 fold, depending on the magnitude of TGFβ1 knockdown. When TGFβ1 is added back to the system using TGFβ1-secreting target cells, the proliferative advantage is eliminated and CTL activity is also reduced. These data suggest that CAR T cells featuring a TGFβ1 knockdown may perform better in a variety of malignant indications alone or in combination with TGFβ receptor modifications.

1. A genetically-modified eukaryotic cell comprising in its genome:
(a) a nucleic acid sequence encoding a transforming growth factor beta-1 (TGFB-1) inhibitory agent; and
(b) a nucleic acid sequence encoding an engineered antigen receptor.

2. The genetically-modified eukaryotic cell of claim 1, wherein said TGFB-1 inhibitory agent reduces expression of TGFB-1 protein in said genetically-modified eukaryotic cell.

3. The genetically-modified eukaryotic cell of claim 1 or 2, wherein said TGFB-1 inhibitory agent is an inhibitory nucleic acid molecule.

4. The genetically-modified eukaryotic cell of claim 3, wherein said inhibitory nucleic acid molecule is an RNA interference molecule.

5. The genetically-modified eukaryotic cell of claim 4, wherein said RNA interference molecule is a short hairpin RNA (shRNA), a small interfering RNA (siRNA), a hairpin siRNA, a microRNA (miRNA), a precursor miRNA, or an miRNA-adapted shRNA (shRNAmiR).

6. The genetically-modified eukaryotic cell of claim 5, wherein said shRNAmiR comprises a nucleic acid sequence having at least 80% sequence identity to a sequence set forth in any one of SEQ ID NOs: 16-19.

7. The genetically-modified eukaryotic cell of claim 5 or 6, wherein said shRNAmiR comprises a nucleic acid sequence set forth in any one of SEQ ID NOs: 16-19.

8. The genetically-modified eukaryotic cell of claim 1 or 2, wherein said TGFB-1 inhibitory agent is an engineered DNA-binding domain comprising a transcriptional repressor domain.

9. The genetically-modified eukaryotic cell of any one of claims 1-8, wherein expression of said TGFB-1 protein by said genetically-modified eukaryotic cell is reduced by about 50% to about 99%, by about 55% to about 99%, by about 60% to about 99%, by about 65% to about 99%, by about 70% to about 99%, by about 75% to about 99%, by about 80% to about 99%, by about 85% to about 99%, by about 90% to about 99%, by about 95% to about 99%, by about 75% to about 95%, by about 80% to about 95%, by about 85% to about 95%, or by about 90% to about 95%, compared to a control cell.

10. The genetically-modified eukaryotic cell of any one of claims 1-9, wherein expression of TGFB-1 protein by said genetically-modified eukaryotic cell is reduced by about 50%, by about 55%, by about 60%, by about 65%, by about 70%, by about 75%, by about 80%, by about 81%, by about 82%, by about 83%, by about 84%, by about 85%, by about 86%, by about 87%, by about 88%, by about 89%, by about 90%, by about 91%, by about 92%, by about 93%, by about 94%, by about 95%, by about 96%, by about 97%, by about 98%, or by about 99%, compared to a control cell.

11. The genetically-modified eukaryotic cell of claim 1, wherein said TGFB-1 inhibitory agent binds TGFB-1 protein produced by said genetically-modified eukaryotic cell.

12. The genetically-modified eukaryotic cell of claim 11, wherein said TGFB-1 inhibitory agent is an antibody, or antibody fragment, having specificity for said TGFB-1 protein.

13. The genetically-modified eukaryotic cell of claim 11, wherein said TGFB-1 inhibitory agent is a soluble TGFB receptor II (TGFBR2).

14. The genetically-modified eukaryotic cell of any one of claims 11-13, wherein said TGFB-1 inhibitory agent is secreted by said genetically-modified eukaryotic cell.

15. The genetically-modified eukaryotic cell of any one of claims 1-14, wherein said nucleic acid encoding said TGFB-1 inhibitory agent is positioned within a target gene of said genetically-modified eukaryotic cell.

16. The genetically-modified eukaryotic cell of claim 15, wherein expression of said target gene is disrupted by insertion of said nucleic acid.

17. The genetically-modified eukaryotic cell of claim 15 or 16, wherein said target gene is a TCR alpha gene, a TCR alpha constant region gene, a TCR beta gene, or a TCR beta constant region gene.

18. The genetically-modified eukaryotic cell of any one of claims 15-17, wherein said target gene is a TCR alpha constant region gene, and said nucleic acid sequence encoding said TGFB-1 inhibitory agent is positioned within a sequence set forth in SEQ ID NO: 20.

19. The genetically-modified eukaryotic cell of claim 18, wherein said nucleic acid sequence encoding said TGFB-1 inhibitory agent is positioned between nucleotides 13 and 14 of SEQ ID NO: 20.

20. The genetically-modified eukaryotic cell of claim 15 or 16, wherein said target gene is a TGFBR2 gene.

21. The genetically-modified eukaryotic cell of claim 20, wherein said genetically-modified eukaryotic cell does not have detectable cell surface expression of TGFBR2.

22. The genetically-modified eukaryotic cell of any one of claims 1-21, wherein said nucleic acid sequence encoding said TGFB-1 inhibitory agent is operably linked to a promoter.

23. The genetically-modified eukaryotic cell of any one of claims 1-22, wherein said engineered antigen receptor is a chimeric antigen receptor, an exogenous T cell receptor, or a TCR fusion construct (TRuC).

24. The genetically-modified eukaryotic cell of any one of claims 1-23, wherein said nucleic acid sequence encoding said TGFB-1 inhibitory agent and said nucleic acid sequence encoding said engineered antigen receptor are positioned in the same gene.

25. The genetically-modified eukaryotic cell of any one of claims 1-24, wherein said genetically-modified eukaryotic cell comprises a polynucleotide comprising said nucleic acid sequence encoding said TGFB-1 inhibitory agent and said nucleic acid sequence encoding said engineered antigen receptor.

26. The genetically-modified eukaryotic cell of claim 25, wherein said polynucleotide comprises a promoter operably linked to said nucleic acid sequence encoding said TGFB-1 inhibitory agent and said nucleic acid sequence encoding said engineered antigen receptor.

27. The genetically-modified eukaryotic cell of claim 25 or 26, wherein said nucleic acid sequence encoding said TGFB-1 inhibitory agent and said nucleic acid sequence encoding said engineered antigen receptor are separated by an IRES or 2A sequence.

28. The genetically-modified eukaryotic cell of claim 25, wherein said polynucleotide comprises a first promoter operably linked to said nucleic acid sequence encoding said TGFB-1 inhibitory agent and a second promoter operably linked to said nucleic acid sequence encoding said engineered antigen receptor.

29. The genetically-modified eukaryotic cell of claim 28, wherein said first promoter and said second promoter are identical.

30. The genetically-modified eukaryotic cell of claim 28, wherein said first promoter and said second promoter are not identical.

31. A genetically-modified eukaryotic cell comprising in its genome:
(a) an inactivated TGFB-1 gene, wherein expression of an encoded TGFB-1 protein is disrupted; and
(b) a nucleic acid sequence encoding an engineered antigen receptor.

32. The genetically-modified eukaryotic cell of claim 31, wherein said engineered antigen receptor is a chimeric antigen receptor, an exogenous T cell receptor, or a TRuC.

33. The genetically-modified eukaryotic cell of claim 31 or 32, wherein said inactivated TGFB-1 gene comprises an insertion or a deletion that disrupts expression of said TGFB-1 protein.

34. The genetically-modified eukaryotic cell of any one of claims 31-33, wherein said inactivated TGFB-1 gene comprises an exogenous polynucleotide that disrupts expression of said TGFB-1 protein.

35. The genetically-modified eukaryotic cell of claim 33 or 34, wherein said insertion or deletion is positioned within an engineered nuclease recognition sequence.

36. The genetically-modified eukaryotic cell of claim 35, wherein said engineered nuclease recognition sequence is an engineered meganuclease recognition sequence, a zinc finger nuclease recognition sequence, a TALEN recognition sequence, a compact TALEN recognition sequence, a CRISPR system nuclease recognition sequence, or a megaTAL recognition sequence.

37. The genetically-modified eukaryotic cell of claim 35 or 36, wherein said engineered nuclease recognition sequence is an engineered meganuclease recognition sequence.

38. The genetically-modified eukaryotic cell of any one of claims 34-37, wherein said exogenous polynucleotide comprises a nucleic acid sequence encoding a polypeptide of interest and/or a nucleic acid sequence encoding an inhibitory nucleic acid molecule.

39. The genetically-modified eukaryotic cell of any one of claims 34-38, wherein said polypeptide of interest is an engineered antigen receptor.

40. The genetically-modified eukaryotic cell of claim 39, wherein said exogenous polynucleotide comprises a promoter operably linked to said nucleic acid sequence encoding said polypeptide of interest and/or said nucleic acid sequence encoding said inhibitory nucleic acid molecule.

41. The genetically-modified eukaryotic cell of any one of claims 31-40, wherein said genetically-modified eukaryotic cell comprises an inactived TCR alpha gene, an inactivated TCR alpha constant region gene, an inactivated TCR beta gene, or an inactivated TCR beta constant region gene.

42. The genetically-modified eukaryotic cell of any one of claims 31-37, wherein said nucleic acid sequence encoding said engineered antigen receptor is positioned within a target gene.

43. The genetically-modified eukaryotic cell of claim 42, wherein expression of a polypeptide encoded by said target gene is disrupted.

44. The genetically-modified eukaryotic cell of claim 42 or 43, wherein said target gene is a TCR alpha gene, a TCR alpha constant region gene, a TCR beta gene, or a TCR beta constant region gene.

45. The genetically-modified eukaryotic cell of any one of claims 42-44, wherein said target gene is a TCR alpha constant region gene, and said nucleic acid sequence encoding said engineered antigen receptor is positioned within a sequence set forth in SEQ ID NO: 20.

46. The genetically-modified eukaryotic cell of claim 45, wherein said nucleic acid sequence encoding said engineered antigen receptor is positioned between nucleotides 13 and 14 of SEQ ID NO: 20.

47. The genetically-modified eukaryotic cell of any one of claims 42-46, wherein said nucleic acid sequence encoding said engineered antigen receptor is operably linked to a promoter.

48. The genetically-modified eukaryotic cell of any one of claims 1-47, wherein said genetically-modified eukaryotic cell comprises in its genome a nucleic acid sequence encoding an HLA class I histocompatibility antigen, alpha chain E (HLA-E) fusion protein.

49. The genetically-modified eukaryotic cell of claim 48, wherein said HLA-E fusion protein comprises an amino acid sequence having at least 80% sequence identity to a sequence set forth in SEQ ID NO: 22.

50. The genetically-modified eukaryotic cell of claim 48, wherein said HLA-E fusion protein comprises an amino acid sequence set forth in SEQ ID NO: 22.

51. The genetically-modified eukaryotic cell of any one of claims 1-50, wherein said genetically-modified eukaryotic cell comprises an inactivated TGFB receptor type II (TGFBR2) gene, wherein expression of an encoded TGFBR2 protein is disrupted.

52. The genetically-modified eukaryotic cell of claim 52, wherein said inactivated TGFBR2 gene comprises an insertion or deletion that disrupts expression of said TGFBR2 protein.

53. The genetically-modified eukaryotic cell of claim 52, wherein said insertion or deletion is positioned within an engineered nuclease recognition sequence.

54. The genetically-modified eukaryotic cell of claim 53, wherein said engineered nuclease recognition sequence is an engineered meganuclease recognition sequence, a zinc finger nuclease recognition sequence, a TALEN recognition sequence, a compact TALEN recognition sequence, a CRISPR system nuclease recognition sequence, or a megaTAL recognition sequence.

55. The genetically-modified eukaryotic cell of claim 53 or 54, wherein said engineered nuclease recognition sequence is an engineered meganuclease recognition sequence.

56. The genetically-modified eukaryotic cell of any one of claims 51-55, wherein said inactivated TGFBR2 gene comprises an exogenous polynucleotide that disrupts expression of said TGFBR2 protein.

57. The genetically-modified eukaryotic cell of claim 56, wherein said exogenous polynucleotide comprises a nucleic acid sequence encoding a polypeptide of interest and/or a nucleic acid sequence encoding an inhibitory nucleic acid molecule.

58. The genetically-modified eukaryotic cell of claim 56 or 57, wherein said wherein said polypeptide of interest is said engineered antigen receptor, or wherein said polypeptide of interest or said inhibitory nucleic acid molecule is a TGFB-1 inhibitory agent.

59. The genetically-modified eukaryotic cell of claim 58, wherein said exogenous polynucleotide comprises a promoter operably linked to said nucleic acid sequence encoding said polypeptide of interest and/or said nucleic acid sequence encoding said inhibitory nucleic acid molecule.

60. The genetically-modified eukaryotic cell of any one of claims 51-59, wherein said genetically-modified eukaryotic cell comprises an inactived TCR alpha gene, an inactivated TCR alpha constant region gene, an inactivated TCR beta gene, or an inactivated TCR beta constant region gene.

61. The genetically-modified eukaryotic cell of any one of claims 1-50, wherein said genetically-modified eukaryotic cell comprises in its genome a nucleic acid sequence encoding a TGFBR2 inhibitory agent.

62. The genetically-modified eukaryotic cell of claim 61, wherein said TGFBR2 inhibitory agent reduces expression of TGFBR2 protein in said genetically-modified eukaryotic cell.

63. The genetically-modified eukaryotic cell of claim 61 or 62, wherein said TGFBR2 inhibitory agent is an inhibitory nucleic acid molecule.

64. The genetically-modified eukaryotic cell of claim 63, wherein said inhibitory nucleic acid molecule is an RNA interference molecule.

65. The genetically-modified eukaryotic cell of claim 64, wherein said RNA interference molecule is a short hairpin RNA (shRNA), a small interfering RNA (siRNA), a hairpin siRNA, a microRNA (miRNA), a precursor miRNA, or an miRNA-adapted shRNA (shRNAmiR).

66. The genetically-modified eukaryotic cell of any one of claims 61-65, wherein expression of said TGFBR2 protein by said genetically-modified eukaryotic cell is reduced by about 50% to about 99%, by about 55% to about 99%, by about 60% to about 99%, by about 65% to about 99%, by about 70% to about 99%, by about 75% to about 99%, by about 80% to about 99%, by about 85% to about 99%, by about 90% to about 99%, by about 95% to about 99%, by about 75% to about 95%, by about 80% to about 95%, by about 85% to about 95%, or by about 90% to about 95%, compared to a control cell.

67. The genetically-modified eukaryotic cell of any one of claims 61-66, wherein expression of TGFBR2 protein by said genetically-modified eukaryotic cell is reduced by about 50%, by about 55%, by about 60%, by about 65%, by about 70%, by about 75%, by about 80%, by about 81%, by about 82%, by about 83%, by about 84%, by about 85%, by about 86%, by about 87%, by about 88%, by about 89%, by about 90%, by about 91%, by about 92%, by about 93%, by about 94%, by about 95%, by about 96%, by about 97%, by about 98%, or by about 99%, compared to a control cell.

68. The genetically-modified eukaryotic cell of any one of claims 61-67, wherein said polynucleotide comprising said nucleic acid sequence encoding said TGFB-1 inhibitory agent comprises said nucleic acid sequence encoding said TGFBR2 inhibitory agent.

69. The genetically-modified eukaryotic cell of any one of claims 61-68, wherein said polynucleotide comprising said nucleic acid sequence encoding said TGFB-1 inhibitory agent and said engineered antigen receptor comprises said nucleic acid sequence encoding said TGFBR2 inhibitory agent.

70. The genetically-modified eukaryotic cell of any one of claims 1-69, wherein said genetically-modified eukaryotic cell is a human cell.

71. The genetically-modified eukaryotic cell of any one of claims 1-70, wherein said genetically-modified eukaryotic cell is an immune cell.

72. The genetically-modified eukaryotic cell of claim 71, wherein said immune cell is a T cell, or a cell derived therefrom, a natural killer (NK) cell, or a cell derived therefrom, a B cell, or a cell derived therefrom, a monocyte, or a cell derived therefrom, or a macrophage, or a cell derived therefrom.

73. The genetically-modified eukaryotic cell of any one of claims 1-70, wherein said genetically-modified eukaryotic cell is a stem cell.

74. The genetically-modified eukaryotic cell of any one of claims 1-70, wherein said genetically-modified eukaryotic cell is an induced-pluripotent stem cell (iPSC).

75. A method of producing a genetically-modified eukaryotic cell, said method comprising introducing a first template nucleic acid into a eukaryotic cell, wherein said first template nucleic acid is inserted into the genome of said eukaryotic cell, wherein said first template nucleic acid comprises a first polynucleotide comprising a nucleic acid sequence encoding a TGFB-1 inhibitory agent, wherein said TGFB-1 inhibitory agent is expressed by said genetically-modified eukaryotic cell.

76. The method of claim 75, wherein said first template nucleic acid is inserted into the genome of said eukaryotic cell by random integration.

77. The method of claim 75 or 76, wherein said first template nucleic acid is introduced into said eukaryotic cell using a recombinant lentivirus.

78. The method of any one of claims 75-77, wherein said eukaryotic cell comprises in its genome a nucleic acid sequence encoding an engineered antigen receptor.

79. The method of claim 78, wherein said nucleic acid sequence encoding said engineered antigen receptor is randomly integrated in the genome.

80. The method of any one of claims 75-77, wherein said first polynucleotide comprises a nucleic acid sequence encoding an engineered antigen receptor.

81. The method of any one of claims 75-80, wherein said genetically-modified eukaryotic cell comprises an inactivated TCR alpha gene, an inactivated TCR alpha constant region gene, an inactivated TCR beta gene, or an inactivated TCR beta constant region gene.

82. The method of claim 75, wherein said eukaryotic cell comprises a nucleic acid sequence encoding an engineered antigen receptor, wherein said nucleic acid sequence encoding said engineered antigen receptor is positioned within a target gene, and wherein expression of said target gene is disrupted.

83. The method of claim 82, wherein said target gene is a TCR alpha gene, a TCR alpha constant region gene, a TCR beta gene, or a TCR beta constant region gene.

84. The method of claim 75, wherein said method comprises introducing into said eukaryotic cell a second template nucleic acid, wherein said second template nucleic acid comprises a second polynucleotide comprising a nucleic acid sequence encoding an engineered antigen receptor, and wherein said second template nucleic acid is integrated into the genome of said eukaryotic cell.

85. The method of claim 84, wherein said second template nucleic acid is randomly integrated in the genome.

86. The method of claim 84 or 85, wherein said second template nucleic acid is introduced into said eukaryotic cell using a recombinant lentivirus.

87. The method of any one of claims 84-86, wherein said genetically-modified eukaryotic cell comprises an inactived TCR alpha gene, an inactivated TCR alpha constant region gene, an inactivated TCR beta gene, or an inactivated TCR beta constant region gene.

88. The method of claim 84, wherein said method comprises introducing into said eukaryotic cell a nucleic acid sequence encoding an engineered nuclease having specificity for a recognition sequence in the genome, wherein said engineered nuclease is expressed by said genetically-modified cell and generates a cleavage site at said recognition sequence, and wherein said second template nucleic acid is inserted into said cleavage site.

89. The method of claim 88, wherein said second template nucleic acid is flanked by homology arms having homology to sequences flanking said cleavage site, and wherein said second template nucleic acid is inserted at said cleavage site by homologous recombination.

90. The method of claim 88 or 89, wherein said recognition sequence is within a target gene, and wherein expression of said target gene is disrupted by said second template nucleic acid.

91. The method of claim 90, wherein said target gene is a TCR alpha gene, a TCR alpha constant region gene, a TCR beta gene, or a TCR beta constant region gene.

92. The method of any one of claims 88-91, wherein said engineered nuclease is an engineered meganuclease, a zinc finger nuclease, a TALEN, a compact TALEN, a CRISPR system nuclease, or a megaTAL.

93. The method of any one of claims 88-92, wherein said engineered nuclease is an engineered meganuclease.

94. The method of any one of claims 84 or 88-93, wherein said second template nucleic acid is introduced into said eukaryotic cell by a recombinant virus.

95. The method of claim 94, wherein said recombinant virus is a recombinant adeno-associated virus (AAV).

96. The method of claim 95, wherein said recombinant AAV has a serotype of AAV6 or AAV2.

97. The method of any one of claims 88-96, wherein said nucleic acid encoding said engineered nuclease is introduced into said eukaryotic cell using an mRNA.

98. The method of any one of claims 78-97, wherein said engineered antigen receptor is a chimeric antigen receptor, an exogenous TCR, or a TRuC.

99. A method of producing a genetically-modified eukaryotic cell, said method comprising introducing a first template nucleic acid into a eukaryotic cell, wherein said first template nucleic acid is inserted into the genome of said eukaryotic cell, wherein said first template nucleic acid comprises a first polynucleotide comprising a nucleic acid sequence encoding a TGFB-1 inhibitory agent, wherein said TGFB-1 inhibitory agent is expressed by said genetically-modified eukaryotic cell.

100. The method of claim 99, wherein said method comprises introducing into the eukaryotic cell a nucleic acid encoding an engineered nuclease having specificity for a recognition sequence in the genome, wherein said engineered nuclease is expressed in said genetically-modified eukaryotic cell and generates a cleavage site at said recognition sequence, and wherein said template nucleic acid is inserted into the genome of said eukaryotic cell at said cleavage site.

101. The method of claim 100, wherein said template nucleic acid is flanked by homology arms having homology to sequences flanking said cleavage site, and wherein said template nucleic acid is inserted at said cleavage site by homologous recombination.

102. The method of any one of claims 99-101, wherein said template nucleic acid is introduced into said eukaryotic cell by a recombinant virus.

103. The method of claim 102, wherein said recombinant virus is a recombinant adeno-associated virus (AAV).

104. The method of claim 103, wherein said recombinant virus has a serotype of AAV6 or AAV2.

105. The method of any one of claims 100-104, wherein said nucleic acid encoding said engineered nuclease is introduced into said eukaryotic cell using an mRNA.

106. The method of any one of claims 100-105, wherein said engineered nuclease is an engineered meganuclease, a zinc finger nuclease, a TALEN, a compact TALEN, a CRISPR system nuclease, or a megaTAL.

107. The method of any one of claims 105-106, wherein said engineered nuclease is an engineered meganuclease.

108. The method of any one of claims 99-107, wherein said first polynucleotide comprises a promoter that is operably linked to said nucleic acid sequence encoding said TGFB-1 inhibitory agent.

109. The method of any one of claims 100-108, wherein said recognition sequence is within a target gene.

110. The method of claim 109, wherein expression of said target gene is disrupted by insertion of said template nucleic acid.

111. The method of claim 109 or 110, wherein said target gene is a TCR alpha gene, a TCR alpha constant region gene, a TCR beta gene, or a TCR beta constant region gene.

112. The method of claim 109 or 110, wherein said target gene is a TGFBR2 gene.

113. The method of any one of claims 99-112, wherein said first polynucleotide comprising said nucleic acid sequence encoding said TGFB-1 inhibitory agent comprises a nucleic acid sequence encoding an engineered antigen receptor.

114. The method of claim 113, wherein said first polynucleotide comprises a promoter operably linked to said nucleic acid sequence encoding said TGFB-1 inhibitory agent and said nucleic acid sequence encoding said engineered antigen receptor.

115. The method of claim 114, wherein said nucleic acid sequence encoding said TGFB-1 inhibitory agent and said nucleic acid sequence encoding said engineered antigen receptor are separated by an IRES or 2A sequence.

116. The method of claim 113, wherein said first polynucleotide comprises a first promoter operably linked to said nucleic acid sequence encoding said TGFB-1 inhibitory agent and a second promoter operably linked to said nucleic acid sequence encoding said engineered antigen receptor.

117. The method of claim 116, wherein said first promoter and said second promoter are identical.

118. The method of claim 116, wherein said first promoter and said second promoter are not identical.

119. The method of any one of claims 99-112, wherein said genetically-modified eukaryotic cell comprises in its genome a nucleic acid sequence encoding an engineered antigen receptor.

120. The method of claim 119, wherein said genetically-modified eukaryotic cell comprises an inactived TCR alpha gene, an inactivated TCR alpha constant region gene, an inactivated TCR beta gene, or an inactivated TCR beta constant region gene.

121. The method of claim 119 or 120, wherein said nucleic acid sequence encoding said engineered antigen receptor is randomly integrated in the genome.

122. The method of claim 119 or 120, wherein said nucleic acid sequence encoding said engineered antigen receptor is positioned within a target gene, and wherein expression of said target gene is disrupted.

123. The method of claim 122, wherein said target gene is a TCR alpha gene, a TCR alpha constant region gene, a TCR beta gene, or a TCR beta constant region gene.

124. The method of claim 122, wherein said target gene is a TGFBR2 gene.

125. The method of any one of claims 99-112, wherein said method comprises introducing into said eukaryotic cell a second template nucleic acid, wherein said second template nucleic acid comprises a second polynucleotide comprising a nucleic acid sequence encoding an engineered antigen receptor, and wherein said second template nucleic acid is integrated into the genome of said eukaryotic cell.

126. The method of claim 125, wherein said second template nucleic acid is randomly integrated in the genome.

127. The method of claim 126, wherein said second template nucleic acid is introduced into said eukaryotic cell using a recombinant lentivirus.

128. The method of any one of claims 125-127, wherein said genetically-modified eukaryotic cell comprises an inactived TCR alpha gene, an inactivated TCR alpha constant region gene, an inactivated TCR beta gene, or an inactivated TCR beta constant region gene.

129. The method of any one of claims 113-128, wherein said engineered antigen receptor is a chimeric antigen receptor, an exogenous T cell receptor, or a TRuC.

130. The method of any one of claims 99-125, wherein said method comprises introducing into said eukaryotic cell a nucleic acid encoding a second engineered nuclease having specificity for a second recognition sequence in the genome, wherein said second engineered nuclease is expressed by said genetically-modified cell and generates a second cleavage site at said second recognition sequence, and introducing into said eukaryotic cell a second template nucleic acid, wherein said second template nucleic acid comprises a second polynucleotide comprising a nucleic acid sequence encoding an engineered antigen receptor, wherein said second template nucleic acid is inserted into said second cleavage site.

131. The method of claim 130, wherein said second template nucleic acid is flanked by homology arms having homology to sequences flanking said cleavage site, and wherein said second template nucleic acid is inserted at said cleavage site by homologous recombination.

132. The method of any one of claims 130 or 131, wherein said second recognition sequence is within a target gene, and wherein expression of said target gene is disrupted by said second template nucleic acid.

133. The method of claim 132, wherein said target gene is a TCR alpha gene, a TCR alpha constant region gene, a TCR beta gene, or a TCR beta constant region gene.

134. The method of any one of claims 130-133, wherein said second engineered nuclease is an engineered meganuclease, a zinc finger nuclease, a TALEN, a compact TALEN, a CRISPR system nuclease, or a megaTAL.

135. The method of any one of claims 130-134, wherein said second engineered nuclease is an engineered meganuclease.

136. The method of any one of claims 130-135, wherein said second template nucleic acid is introduced into said eukaryotic cell by a recombinant virus.

137. The method of claim 136, wherein said recombinant virus is a recombinant AAV.

138. The method of claim 137, wherein said recombinant AAV has a serotype of AAV6 or AAV2.

139. The method of any one of claims 130-138, wherein said second polynucleotide comprises a promoter operably linked to said nucleic acid sequence encoding said engineered antigen receptor.

140. The method of any one of claims 130-139, wherein said engineered antigen receptor is a chimeric antigen receptor, an exogenous T cell receptor, or a TRuC.

141. The method of any one of claims 75-140, wherein said TGFB-1 inhibitory agent reduces expression of TGFB-1 protein in said genetically-modified eukaryotic cell.

142. The method of claim 141, wherein said TGFB-1 inhibitory agent is an inhibitory nucleic acid molecule.

143. The method of claim 142, wherein said inhibitory nucleic acid molecule is an RNA interference molecule.

144. The method of claim 143, wherein said RNA interference molecule is a short hairpin RNA (shRNA), a small interfering RNA (siRNA), a hairpin siRNA, a microRNA (miRNA), a precursor miRNA, or an miRNA-adapted shRNA (shRNAmiR).

145. The method of claim 144, wherein said shRNAmiR comprises:
(a) a guide strand comprising a nucleic acid sequence set forth in SEQ ID NO: 8 and a passenger strand comprising a nucleic acid sequence set forth in SEQ ID NO: 9;
(b) a guide strand comprising a nucleic acid sequence set forth in SEQ ID NO: 10 and a passenger strand comprising a nucleic acid sequence set forth in SEQ ID NO: 11;
(c) a guide strand comprising a nucleic acid sequence set forth in SEQ ID NO: 12 and a passenger strand comprising a nucleic acid sequence set forth in SEQ ID NO: 13; or
(d) a guide strand comprising a nucleic acid sequence set forth in SEQ ID NO: 14 and a passenger strand comprising a nucleic acid sequence set forth in SEQ ID NO: 15.

146. The method of claim 144 or 145, wherein said shRNAmiR comprises a nucleic acid sequence having at least 80% sequence identity to a sequence set forth in any one of SEQ ID NOs: 16-19.

147. The method of any one of claims 144-146, wherein said shRNAmiR comprises a nucleic acid sequence set forth in any one of SEQ ID NOs: 16-19.

148. The method of claim 141, wherein said TGFB-1 inhibitory agent is an engineered DNA-binding domain comprising a transcriptional repressor domain.

149. The method of any one of claims 141-148, wherein expression of said TGFB-1 protein by said genetically-modified eukaryotic cell is reduced by about 50% to about 99%, by about 55% to about 99%, by about 60% to about 99%, by about 65% to about 99%, by about 70% to about 99%, by about 75% to about 99%, by about 80% to about 99%, by about 85% to about 99%, by about 90% to about 99%, by about 95% to about 99%, by about 75% to about 95%, by about 80% to about 95%, by about 85% to about 95%, or by about 90% to about 95%, compared to a control cell.

150. The method of any one of claims 141-149, wherein expression of TGFB-1 protein by said genetically-modified eukaryotic cell is reduced by about 50%, by about 55%, by about 60%, by about 65%, by about 70%, by about 75%, by about 80%, by about 81%, by about 82%, by about 83%, by about 84%, by about 85%, by about 86%, by about 87%, by about 88%, by about 89%, by about 90%, by about 91%, by about 92%, by about 93%, by about 94%, by about 95%, by about 96%, by about 97%, by about 98%, or by about 99%, compared to a control cell.

151. The method of any one of claims 75-140, wherein said TGFB-1 inhibitory agent binds TGFB-1 protein produced by said genetically-modified eukaryotic cell.

152. The method of claim 151, wherein said TGFB-1 inhibitory agent is an antibody, or antibody fragment, having specificity for said TGFB-1 protein.

153. The method of claim 151, wherein said TGFB-1 inhibitory agent is a soluble TGFBR2 protein.

154. The method of any one of claims 151-153, wherein said TGFB-1 inhibitory agent is secreted by said genetically-modified eukaryotic cell.

155. A method of producing a genetically-modified eukaryotic cell, said method comprising introducing into a eukaryotic cell a nucleic acid encoding a first engineered nuclease having specificity for a first recognition sequence in the TGFB-1 gene, wherein said first engineered nuclease is expressed by said eukaryotic cell and generates a first cleavage site at said recognition sequence.

156. The method of claim 155, wherein said first cleavage site is repaired by non-homologous end joining, resulting in an insertion or deletion that disrupts expression of TGFB-1 protein.

157. The method of claim 155, wherein said method comprises introducing into the eukaryotic cell a first template nucleic acid comprising a first polynucleotide, wherein said first template nucleic acid is inserted into the genome of said eukaryotic cell at said first cleavage site in said TGFB-1 gene, and wherein insertion of said first template nucleic acid disrupts expression of TGFB-1 protein.

158. The method of any one of claims 155-157, wherein said first engineered nuclease is an engineered meganuclease, a zinc finger nuclease, a TALEN, a compact TALEN, a CRISPR system nuclease, or a megaTAL.

159. The method of any one of claims 155-158, wherein said first engineered nuclease is an engineered meganuclease.

160. The method of any one of claims 155-159, wherein said first template nucleic acid is flanked by homology arms having homology to sequences flanking said first cleavage site in said TGFB-1 gene, and wherein said first template nucleic acid is inserted at said first cleavage site by homologous recombination.

161. The method of any one of claims 157-160, wherein said first template nucleic acid is introduced into said eukaryotic cell using a recombinant virus.

162. The method of claim 161, wherein said recombinant virus is a recombinant AAV.

163. The method of claim 162, wherein said recombinant AAV has a serotype of AAV6 or AAV2.

164. The method of any one of claims 157-163, wherein said first polynucleotide comprises a nucleic acid sequence encoding a polypeptide of interest or an inhibitory nucleic acid.

165. The method of any one of claims 157-163, wherein said genetically-modified eukaryotic cell comprises an inactived TCR alpha gene, an inactivated TCR alpha constant region gene, an inactivated TCR beta gene, or an inactivated TCR beta constant region gene.

166. The method of any one of claims 155-165, wherein said genetically-modified eukaryotic cell comprises in its genome a nucleic acid sequence encoding an engineered antigen receptor.

167. The method of claim 166, wherein said nucleic acid sequence encoding said engineered antigen receptor is randomly integrated in the genome.

168. The method of claim 166 or 167, wherein said genetically-modified eukaryotic cell comprises an inactived TCR alpha gene, an inactivated TCR alpha constant region gene, an inactivated TCR beta gene, or an inactivated TCR beta constant region gene.

169. The method of claim 166, wherein said nucleic acid sequence encoding said engineered antigen receptor is positioned within a TCR alpha gene, a TCR alpha constant region gene, a TCR beta gene, or a TCR beta constant region gene.

170. The method of any one of claims 157-169, wherein said polypeptide of interest encoded by said first polynucleotide is an engineered antigen receptor.

171. The method of any one of claims 155-170, wherein said method comprises introducing into said eukaryotic cell a second template nucleic acid comprising a second polynucleotide comprising a nucleic acid sequence encoding an engineered antigen receptor, wherein said second template nucleic acid is inserted into the genome of said eukaryotic cell.

172. The method of claim 171, wherein said second template nucleic acid is inserted into the genome of said eukaryotic cell by random integration.

173. The method of claim 172, wherein said second template nucleic acid is introduced into said eukaryotic cell using a recombinant lentivirus.

174. The method of any one of claims 171-173, wherein said genetically-modified eukaryotic cell comprises an inactived TCR alpha gene, an inactivated TCR alpha constant region gene, an inactivated TCR beta gene, or an inactivated TCR beta constant region gene.

175. The method of any one of claims 171-174, wherein said method comprises introducing into said eukaryotic cell a nucleic acid encoding a second engineered nuclease, wherein said second engineered nuclease has specificity for a second recognition sequence in the genome of said eukaryotic cell, wherein said second engineered nuclease is expressed in said eukaryotic cell and generates a second cleavage site at said second recognition sequence, and wherein said second template nucleic acid is inserted into the genome of said eukaryotic cell at said second cleavage site.

176. The method of claim 175, wherein said second template nucleic acid is flanked by homology arms having homology to sequences flanking said second cleavage site, and wherein said second template nucleic acid is inserted at said second cleavage site by homologous recombination.

177. The method of claim 175 or 176, wherein said second template nucleic acid is introduced into said eukaryotic cell using a recombinant virus.

178. The method of claim 177, wherein said recombinant virus is a recombinant AAV.

179. The method of claim 178, wherein said recombinant AAV has a serotype of AAV6 or AAV2.

180. The method of any one of claims 175-179, wherein said second engineered nuclease is an engineered meganuclease, a zinc finger nuclease, a TALEN, a compact TALEN, a CRISPR system nuclease, or a megaTAL.

181. The method of any one of claims 175-180, wherein said second engineered nuclease is an engineered meganuclease.

182. The method of any one of claims 175-181, wherein said second recognition sequence is within a target gene.

183. The method of claim 182, wherein expression of said target gene is disrupted by insertion of said second template nucleic acid.

184. The method of claim 182 or 183, wherein said target gene is a TCR alpha gene, a TCR alpha constant region gene, a TCR beta gene, or a TCR beta constant region gene.

185. The method of any one of claims 166-184, wherein said engineered antigen receptor is a chimeric antigen receptor, an exogenous T cell receptor, or a TRuC.

186. The method of any one of claims 75-185, wherein said genetically-modified eukaryotic cell is a human cell.

187. The method of any one of claims 75-186, wherein said genetically-modified eukaryotic cell is an immune cell.

188. The method of claim 187, wherein said immune cell is a T cell, or a cell derived therefrom, a natural killer (NK) cell, or a cell derived therefrom, a B cell, or a cell derived therefrom, a monocyte, or a cell derived therefrom, or a macrophage, or a cell derived therefrom.

189. The method of any one of claims 75-186, wherein said genetically-modified eukaryotic cell is a stem cell.

190. The method of any one of claims 75-186, wherein said genetically-modified eukaryotic cell is an iPSC.

191. A genetically-modified eukaryotic cell prepared by the method of any one of claims 75-190.

192. A population of cells comprising a plurality of said genetically-modified eukaryotic cell of any one of claims 1-74 and 191.

193. A pharmaceutical composition comprising said genetically-modified eukaryotic cell of any one of claims 1-74 and 191.

194. A pharmaceutical composition comprising said population of cells of claim 192.

195. A method for reducing the number of target cells in a subject in need thereof, said method comprising administering to said subject an effective amount of said population of cells of claim 192, or said pharmaceutical composition of claim 193, wherein said genetically-modified eukaryotic cells express an engineered antigen receptor having specificity for an antigen present on said target cells.

196. The method of claim 195, wherein said method is a method of immunotherapy.

197. The method of claim 195 or 196, wherein said target cells are cancer cells.

198. The method of any one of claims 195-197, wherein said method reduces the size of said cancer.

199. The method of any one of claims 195-198, wherein said method eradicates said cancer in said subject.


SEQUENCE LISTING (25 sequences)

SEQ ID NO: 1 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 2 - protein, 9 aa, Chlamydomonas reinhardtii
LAGLIDADG

SEQ ID NO: 3 - DNA, 19 bp, Artificial sequence
  Synthesized
tagtgaagccacagatgta

SEQ ID NO: 4 - DNA, 118 bp, Artificial sequence
  Synthesized
ctgtttgaatgaggcttcagtactttacagaatcgttgcctgcacatcttggaaacacttgctgggattacttcgacttcttaacccaacagaaggctcgagaaggtatattgctgtt

SEQ ID NO: 5 - DNA, 11 bp, Artificial sequence
  Synthesized
gacagtgagcg

SEQ ID NO: 6 - DNA, 11 bp, Artificial sequence
  Synthesized
tgcctactgcc

SEQ ID NO: 7 - DNA, 121 bp, Artificial sequence
  Synthesized
tcggacttcaaggggctagaattcgagcaattatcttgtttactaaaactgaataccttgctatctctttgatacatttttacaaagctgaattaaaatggtataaattaaatcactttgc

SEQ ID NO: 8 - DNA, 22 bp, Artificial sequence
  Synthesized
ttacagcgagatgtcatttccc

SEQ ID NO: 9 - DNA, 22 bp, Artificial sequence
  Synthesized
aggaaatgacatctcgctgtaa

SEQ ID NO: 10 - DNA, 22 bp, Artificial sequence
  Synthesized
tcatagatttcgttgtgggttt

SEQ ID NO: 11 - DNA, 22 bp, Artificial sequence
  Synthesized
caacccacaacgaaatctatga

SEQ ID NO: 12 - DNA, 22 bp, Artificial sequence
  Synthesized
atgctgtgtgtactctgcttga

SEQ ID NO: 13 - DNA, 22 bp, Artificial sequence
  Synthesized
ccaagcagagtacacacagcat

SEQ ID NO: 14 - DNA, 22 bp, Artificial sequence
  Synthesized
gtagtgaacccgttgatgtcca

SEQ ID NO: 15 - DNA, 22 bp, Artificial sequence
  Synthesized
cggacatcaacgggttcactac

SEQ ID NO: 16 - DNA, 324 bp, Artificial sequence
  Synthesized
ctgtttgaatgaggcttcagtactttacagaatcgttgcctgcacatcttggaaacacttgctgggattacttcgacttcttaacccaacagaaggctcgagaaggtatattgctgttgacagtgagcgaggaaatgacatctcgctgtaatagtgaagccacagatgtattacagcgagatgtcatttccctgcctactgcctcggacttcaaggggctagaattcgagcaattatcttgtttactaaaactgaataccttgctatctctttgatacatttttacaaagctgaattaaaatggtataaattaaatcactttgc

SEQ ID NO: 17 - DNA, 324 bp, Artificial sequence
  Synthesized
ctgtttgaatgaggcttcagtactttacagaatcgttgcctgcacatcttggaaacacttgctgggattacttcgacttcttaacccaacagaaggctcgagaaggtatattgctgttgacagtgagcgcaacccacaacgaaatctatgatagtgaagccacagatgtatcatagatttcgttgtgggttttgcctactgcctcggacttcaaggggctagaattcgagcaattatcttgtttactaaaactgaataccttgctatctctttgatacatttttacaaagctgaattaaaatggtataaattaaatcactttgc

SEQ ID NO: 18 - DNA, 324 bp, Artificial sequence
  Synthesized
ctgtttgaatgaggcttcagtactttacagaatcgttgcctgcacatcttggaaacacttgctgggattacttcgacttcttaacccaacagaaggctcgagaaggtatattgctgttgacagtgagcgccaagcagagtacacacagcattagtgaagccacagatgtaatgctgtgtgtactctgcttgatgcctactgcctcggacttcaaggggctagaattcgagcaattatcttgtttactaaaactgaataccttgctatctctttgatacatttttacaaagctgaattaaaatggtataaattaaatcactttgc

SEQ ID NO: 19 - DNA, 324 bp, Artificial sequence
  Synthesized
ctgtttgaatgaggcttcagtactttacagaatcgttgcctgcacatcttggaaacacttgctgggattacttcgacttcttaacccaacagaaggctcgagaaggtatattgctgttgacagtgagcgcggacatcaacgggttcactactagtgaagccacagatgtagtagtgaacccgttgatgtccatgcctactgcctcggacttcaaggggctagaattcgagcaattatcttgtttactaaaactgaataccttgctatctctttgatacatttttacaaagctgaattaaaatggtataaattaaatcactttgc

SEQ ID NO: 20 - DNA, 22 bp, Homo sapiens
tggcctggagcaacaaatctga

SEQ ID NO: 21 - DNA, 22 bp, Homo sapiens
accggacctcgttgtttagact

SEQ ID NO: 22 - protein, 325 aa, Artificial sequence
  Synthesized
VMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHL

SEQ ID NO: 23 - DNA, 164 bp, Artificial sequence
  Synthesized
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt

SEQ ID NO: 24 - DNA, 212 bp, Artificial sequence
  Synthesized
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 25 - protein, 354 aa, Artificial sequence
  Synthesized
MNTKYNKEFLLYLAGFVDGDGSIYAVIYPHQRAKFKHFLKLLFTVSQSTKRRWFLDKLVDEIGVGYVYDLPRTSEYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIRPRQGSKFKHRLTLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLDSLSEKKKSSP